(19) United States
(12) Patent Application Publication
(10) Pub. No.: US 2002/0088014 A1
(43) Pub. Date: Jul. 4, 2002

(54) Minimal adenovirus mediated recombinant vaccine

(76) Inventors: Xiangming Fang, San Diego, CA (US); Scott Gallichan, San Diego, CA (US); Wei-Wei Zhang, San Diego, CA (US); Flossie Wong-Staal, San Diego, CA (US); Sybille Sauter, Del Mar, CA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/974,206

(22) Filed: Oct. 10, 2001

Related U.S. Application Data
(63) Continuation-in-part of application No. 08/866,403, filed on May 30, 1997.
(63) Continuation-in-part of application No. 08/791,218, filed on Jan. 31, 1997, now abandoned.
(63) Continuation-in-part of application No. 08/658,961, filed on May 31, 1996.
(60) Provisional application No. 60/239,224, filed on Oct. 10, 2000.
(60) Provisional application No. 60/241,625, filed on Oct. 19, 2000.

(51) Int. Cl. A01K 67/00; C12N 15/867; C12N 15/861; C12N 7/00; C12N 7/01

(52) U.S. Cl. 800/8; 435/320.1; 435/456; 435/235.1

(57) ABSTRACT

This invention is related to adenoviral (Ad) vectors and their applications in the field of genetic medicine, including, but not limited to, gene vaccination, gene transfer, gene therapy, and the like. More specifically, this invention is related to the Ad vectors that carry the minimal cis-element of the Ad genome (minimal Ad vector) and are capable of delivering about 36 kb to about 38 kb of heterologous DNA. The generation and propagation of the minimal Ad vectors require trans-complementation of a packaging-attenuated and replication-defective helper Ad (helper) in an Ad helper cell line.

This invention further comprises minimal adenoviral vectors for use in the treatment or prevention of disease or other medical conditions, methodologies for generating such vectors and animal test systems for in vivo evaluation of such Ad vectors. More specifically, this invention describes HIV and/or HPV Ad vectors that contain minimal cis-elements of the Ad genome and comprise HIV and/or HPV nucleic acid sequence with other supporting and/or complementing nucleic acid elements up to about 36 kb to about 38 kb. The HIV and/or HPV minimal Ad may be generated and preferentially amplified through the assistance of a packaging-attenuated helper Ad and a helper cell line. This invention also discloses designs and methods for testing such minimal Ad vectors in vivo.

### CROSS-REFERENCE TO RELATED APPLICATIONS

This application claims priority from U.S. Provisional App. No. 60/239,224, filed Oct. 10, 2000, and from U.S. Provisional App. No. 60/241,625, filed Oct. 19, 2000. This application is a continuation-in-part of U.S. app. Ser. No. 08/866,403, filed May 30, 1997, which is in turn a continuation-in-part of U.S. app. Ser. No. 08/791,218, filed Jan. 31, 1997, which is in turn a continuation-in-part of U.S. App. Ser. No. 08/658,961, filed May 31, 1996. This application is related to the following published International Patent Applications (and to any applications from which these applications claim priority): Int. App. No. PCT/US97/10218, filed May 30, 1997 and published Dec. 4, 1997 as WO 97/45550; Int. App. No. PCT/US97/23685, filed Dec. 19, 1997 and published Jul. 30, 1998 as WO 98/32860; Int. app. No. PCT/US98/01301, filed Jan. 23, 1998 and published Aug. 13, 1998 as WO 98/35028; Int. App. No. PCT/US98/03473, filed Feb. 23, 1998 and published Sep. 11, 1998 as WO 98/39411; and Int. App. No. PCT/US98/10330, filed May 19, 1998 and published Dec. 3, 1998 as WO 98/54345. All patents, published and unpublished patent applications as well as any other scientific, technical and general writings referred to herein are incorporated by reference to the extent that they are not contradictory.

### BACKGROUND OF THE INVENTION

1. Technical Field of the Invention

This invention is related to adenoviral (Ad) vectors and their applications in the field of genetic medicine, including gene vaccination, gene therapy, and/or gene transfer. More specifically, this invention is related to Ad vectors that carry minimal cis-elements of the Ad genome (called MAXIMUM-Ad™, Max-Ad, Maxi-Ad, or minimal Ad) and are capable of delivering transgenes and/or heterologous DNA up to approximately 36 kb. The generation and propagation of MAXIMUM-Ad™ vectors may require trans-complementation of a packaging-attenuated and replication-defective helper Ad (helper) in an Ad helper cell line.

This invention further comprises a methodology for generating minimal adenoviral vectors for use in the treatment of infection, including, but not limited to HIV and/or HPV infection. The invention further comprises animal test systems for in vivo evaluation of such Ad vectors. More specifically, this invention describes HIV and/or HPV Ad vectors that may comprise minimal cis-elements of the Ad genome and comprise HIV and/or HPV DNA sequences with other supporting DNA elements up to 36 kb. The vectors of the present invention may further comprise immuno-modulatory DNA sequences. The HIV and/or HPV minimal Ad may be generated and may be further amplified through the assistance of a packaging-attenuated helper Ad and/or a helper cell line. This invention also discloses designs and methods for testing the minimal Ad vectors in vivo.

2. Description of the Related Art

An important issue in the development of genetic medicine is the development of preferred gene delivery systems. A preferred system of gene delivery must possess several properties that are currently unavailable in a single gene therapy vector. A preferred vector must retain adequate capacity to accommodate large or multiple transgenes including regulatory elements and be amenable to simple manipulation and scale-up for manufacturing. Such a vector must also be safe and demonstrate low toxicity as well as demonstrate highly efficient and selective delivery of transgenes into target cells or tissues. Finally, such a vector must be capable of supporting appropriate retention, expression, and regulation of the transgenes in target cells. The present invention encompasses a novel design of a high-capacity and highly-efficient Ad vector system and is focused on resolving the issues and concerns of those skilled in the art regarding an preferred gene delivery system.

Retroviral vectors were among the first to be studied for use in gene therapy (Miller, A. D. & Rosman, G. J. (1989) “Improved retroviral vectors for gene therapy and expression” BioTechniques, vol. 7, pp. 980-990; Mulligan, R. C. (1993) “The basic science of gene therapy” Science, vol. 260, pp. 926-932). The size capacity for insertion of exogenous DNA into retroviral vectors is limited to approximately 7.5 kb. Adenoviral vectors may also used in gene therapy and genetic vaccination protocols. Recently, an E1-substituted adenoviral vector comprising a B-domain deleted FVIII cDNA under control of the murine albumin promoter has been utilized to achieve therapeutic levels of human FVIII expression in mice and dogs (Connelly, S. et al. (1995) “In vivo gene delivery and expression of physiological levels of functional human factor VIII in mice” Hu. Gene Ther., vol. 6, pp. 185-193; Connelly, S. et al. (1996) “High-level tissue-specific expression of functional human factor VIII in mice.” Hum. Gene Ther., vol. 7, pp.183-195; Connelly, S. et al. (1996) “Complete short-term correction of canine hemophilia A by in vivo gene therapy” Blood, vol. 88, pp. 3846-3853). Adenoviral vectors are preferred for delivery of proteins, peptides, or non-coding nucleic acid sequences due to their large capacity for exogenous DNA (up to about 36 kb).

Some difficulties, however, are associated with adenoviral gene delivery. For instance, Ad does not normally integrate into the host cell genome. To sustain long-term transgene expression, an Ad vector may preferably include elements required for integration into the host cell genome or other mechanisms of DNA retention. Additionally, the immune response mediated against the adenoviral vector makes re-administration of the vector difficult (Yang, Y. et al. (1994) “Cellular immunity to viral antigen limits E1-deleted adenoviruses for gene therapy” Proc. Natl. Acad Sci. USA, vol. 91, pp. 4407-4411). The minimal Ad vectors of the present invention have eliminated most adenovirus DNA sequences from the adenoviral vector carrying the transgene. Thus, at least partially eliminating a detrimental immune response that may be raised by Ad gene expression in the host cell, which may contribute to the decline of transgene expression.

An adenoviral vector that allows for a large heterologous DNA insert has been described in international patent application WO 96/33280 (International Patent Application number PCT/US96/05310, published Oct. 24, 1996 as WO 96/33280). That vector, however, does not provide elements for integration into the target cell genome or for episomal maintenance of the vector upon entry into a target cell.

The present invention provides elements that allow for retention of the delivered transgene in the host cell, either by integration into the target cell genome or by maintenance as an episomal nucleic acid. One method by which this may be accomplished by the present invention includes facilitation of integration of the transgene into the host cell genome using viral integration mechanisms. The adeno-associated virus (AAV) genome has the capability of integrating into the DNA of cells which it infects and is the only example of an exogenous DNA that integrates at a specific site, AAVS1 at 19q13.3-qter, in the human genome (Kotin, R. M. et al. (1992) “Characterization of a preferred site on human chromosome 19q for integration of adeno-associated virus DNA by non-homologous recombination.” EMBO J., vol. 11, pp. 5071-5078; International Patent Application number PCT/US96/14312, published Mar. 13, 1997 as WO 97/09442). The minimal elements for AAV integration are the inverted terminal repeat (ITR) sequences and a functional Rep 78/68 protein. The present invention incorporates these integration elements for integration of the transgene into the host cell genome for sustained transgene expression. The present invention also provides an adenoviral vector capable of homologous recombination into the genome of a target cell, another significant advantage over adenoviral vectors currently available to one skilled in the art. The present invention also provides elements that allow episomal replication of the transgene.

In vitro model systems have been developed to detect site-specific integration of AAV or AAV-based vectors in immortalized cell lines (Kotin, R. et al. (1990) “Site-specific integration by adeno-associated virus” Proc. Natl Acad. Sci. USA, vol. 87, pp. 2211-2215; Samulski, J. et al. (1991) “Targeted integration of adeno-associated virus (AAV) into human chromosome 19” EMBO J., vol. 10, pp. 3941-3950; Shelling, A. & Smith, M. (1994) “Targeted integration of transfected and infected adeno-associated virus vectors containing the neomycin resistance gene” Gene Ther., vol. 1, pp. 165-169; International Patent Application numver PCT/GB96/02061, published Mar. 6, 1997 as WO 97/08330), in episomal systems (Giraud, C. et al. (1994) “Site-specific integration by adeno-associated virus is directed by a cellular sequence” Proc. Natl Acad. Sci. USA, vol. 91, pp. 10039-10043; Giraud, C. et al. (1995) “Recombinant junctions formed by site-specific integration of adeno-associated virus into an episome” J. Virol., vol. 69, pp. 6917-6924; Linden, R. et al. (1996) “The recombination signals for adeno-associated virus site-specific integration” Proc. Natl. Acad. Sci. USA, vol. 93, pp. 7966-7972), and in cell-free extracts (Urcelay, E. et al. (1995) “Asymmetric replication in vitro from a human sequence element is dependent on adeno-associated virus rep protein” J. Virol., vol. 69, pp. 2038-2046). A comparison of transduction efficiencies of AAV using either primary human cells or immortalized cell lines, demonstrated that the transduction efficiency was 10-60 times greater in immortalized human cells than in primary cells (Halbert, C. et al. (1995) “Adeno-associated virus vectors transduce primary cells much less efficiently than immortalized cells” J. Virol., vol. 69, pp. 1473-1479). These results stress the importance of using primary cells, or even better, in vivo model systems, to accurately evaluate AAV vectors for gene therapy applications. However, to date, no in vivo animal model system has been developed to detect site-specific integration. An animal model having the human AAVS1 sequence incorporated into its genome is provided in the present invention. This animal model will be useful for evaluation of vectors containing the AAV integration mechanism, not only to test site-specific integration, but also in vivo gene delivery, gene transduction efficiency, tissue distribution, and duration of gene expression.

The minimal Ad vector system of the present invention was developed based on two major findings: 1) the discovery of an Ad-SV40 hybrid (Gluzman, Y. & Van Doren, K. (1983) “Palindromic adenovirus type 5-simian virus 40 hybrid”. J Virol., vol. 45, pp. 91-103) in which the majority of the viral genome was replaced by SV40 sequences but was able to be processed and packaged due to the presence of Ad ITR and packaging elements; and, 2) that Ad packaging may be attenuated by partial deletion of the packaging signal (Grable, M., & Hearing, P. (1992) “Cis and trans requirements for the selective packaging of adenovirus type 5 DNA” J. Virol., vol. 66, pp. 723-731). Other adenoviral vector packaging systems based on incorporation of minimal cis elements for packaging and genome replication are under development (Fisher, K. J. et al. (1996) “Recombinant adenovirus deleted of all viral genes for gene therapy of cystic fibrosis” Virology, vol. 217, pp. 11-22; Haecker, S. E. et al. (1996) “In vivo expression of full-length human dystrophin from adenoviral vectors deleted of all viral genes” Hum. Gene Ther., vol. 7, pp. 1907-1904; Kochanek, S. et al. (1996) “A new adenoviral vector: Replacement of all viral coding sequences with 28 kb of DNA independently expressing both full-length dystrophin and β-galactosidase” Proc. Natl. Acad. Sci. USA, vol. 93, pp. 5731-5736).

### BRIEF SUMMARY OF THE INVENTION

It is an object of the present invention to provide modified adenoviral vectors comprising: 1) a large capacity for insertion of approximately 36 kb heterologous DNA, wherein such sequences may include, but are not limited to, one or more coding and/or non-coding sequences, one or more elements for controlling transgene expression, one or more elements for assisting in integration of exogenous DNA into a target cell genome, and/or one or more elements for the maintenance of a vector in an episomal form within a target cell; 2) cognate helper Ad vectors designed to support propagation of the minimal Ad vectors and having a manipulated packaging signal such that within a host producer cell the minimal Ad vector is packaged at a greater frequency than the helper Ad vector; and, 3) helper cell lines designed to support propagation of both the minimal Ad vectors and the helper Ad vectors that may also serve to control transgene expression during viral propagation and selectively attenuate packaging of the helper Ad genome.

It is an objective of the present invention to provide minimal Ad vectors able to accommodate an insert capacity of up to approximately 36-37 kb, sufficient for delivery of one or more nucleic acids encoding a protein having a large coding sequence, non-coding sequences, and/or control and maintenance sequences.

In one embodiment, the present invention provides minimal Ad vectors as an isolated DNA molecule having the elements necessary for replication, packaging and sustained gene expression such as an inverted terminal repeat (ITR), a packaging signal, a transcriptional control region, an effector or reporter gene, and either a genomic integration sequence or an episomal maintenance sequence, all operatively associated for generating an infectious, replication-defective recombinant adenoviral vector wherein the remaining portion of said DNA molecule does not encode an adenoviral protein. In one example of such a vector, a minimal Ad vector comprising HIV and/or HPV DNA sequences is provided.

It is yet another objective of the present invention to provide a vector having the elements required to extend the duration of expression of a transgene following introduction into a target cell. The present invention thus provides a vector having an element that may aid in stabilization of transgene expression in the target cell as an episome or by facilitating integration of the introduced gene into the cell genome. In one embodiment, the present invention provides either a genomic integration element or an episomal maintenance element within the minimal Ad vector. An example of such a system provides a minimal Ad vector including integration elements of AAV. Another example of such a system provides a homologous recombination arm within the minimal Ad vector.

It is yet another objective of the present invention to provide reagents and methodologies for producing minimal Ad vectors using a packaging-attenuated and replication-defective helper Ad in combination with an E1-complementing Ad helper cell line. In one embodiment, an E1-deleted helper Ad genome having an altered packaging signal such that the E1-deleted helper Ad genome is packaged at a lower frequency than the wild-type helper Ad genome is provided. In another embodiment, a helper or producer cell is provided as a cell stably transfected with an Ad E1 gene sequence that has no overlapping sequence with the genome of an E1-deleted helper Ad genome is provided. And, in yet another embodiment, a method of generating a recombinant adenoviral vector by co-transfecting the helper or producer cell with a minimal Ad vector and an Ad-helper genome and/or infecting the cell with an Ad helper virus, and preparing a cell-free lysate of said producer cell is provided. The cell-free lysate thus prepared contains infective, replication-impaired recombinant adenoviral vector particles, the majority of which include the minimal Ad vector DNA.

It has also proven difficult for those skilled in the art to analyze the transduction efficiency and targeting of AAV to its integration site, AAVS1, in an animal model system in vivo. It is yet another objective of the present invention is to provide an animal model system for assessment of targeted integration as directed by the AAV integration mechanisms incorporated into the minimal Ad vectors of the present invention. As an example, the present invention provides a methodology for the generation of transgenic mice harboring the human AAVS1 integration sequence within their genome. Following injection of a minimal Ad vector into such an animal, targeted integration of the transgene into the AAVS1 site may be evaluated.

Another object of the present invention is to provide minimal adenovirus vectors comprising HIV and/or HPV sequences, non-coding sequences, immunomodulatory sequences, and/or other nucleic acid sequences.

The present invention, therefore, provides the reagents and methodologies needed to overcome many of the difficulties associated with gene therapy vectors that have been encountered by those skilled in the art. The objectives described above as well as other objectives of the present invention will be understood in light of the detailed description of the invention provided below.

### BRIEF DESCRIPTION OF THE SEVERAL VIEWS OF THE DRAWINGS

FIG. 1: Protypical minimal adenoviral vaccine vectors: shown is a possible prototype minimal adenoviral vector useful for vaccine applications.

FIG. 2. Minimal Ad/HIV Constructs: shown are embodiments of minimal adenoviral vectors comprising HIV sequences.

FIG. 3. Minimal Ad/HPV Constructs: shown are embodiments of minimal adenoviral vectors comprising HPV sequences.

### DETAILED DESCRIPTION OF THE INVENTION

The present invention comprises three components useful in generating adenoviral vectors capable of delivering prophylactic and/or therapeutic nucleic acid sequences including, but not limited to, HIV and/or HPV sequences to a target cell or tissue in vivo. The components consist of a minimal viral genome, a helper virus, and a helper cell line. The helper virus and helper cell line are utilized to package a minimal viral genome into viral particles for use in genetic medicine applications, including, but not limited to, genetic vaccination, gene therapy and/or gene delivery. The minimal viruses generated using such a system have similar tropism and host range as the adenoviral strain from which the helper virus was derived.

The present invention further provides modifications of a minimal Ad vector to comprise elements derived from the adeno-associated virus (AAV). The elements are those having the ability to promote integration of genetic material into a host cell genome. In the present invention, the elements are utilized to promote integration of a reporter or effector gene of a minimal Ad vector into a host cell genome. In this manner, expression of the gene is observed in the host cell or tissue for a longer period of time than that of a conventional adenoviral vector.

The present invention further provides minimal Ad vectors that comprise elements for maintaining such vectors as an episome in a host cell or tissue to prolong expression of the delivered gene or genes. It has been determined that limited replication of the viral genome of E1-deleted viruses in a host cell allows for longer term expression of a gene of interest as compared to those genomes that are not able to replicate (Lieber, et al. (1996) “Recombinant adenoviruses with large deletions generated by Cre-mediated excision exhibit different biological properties compared with first-generation vectors in vitro and in vivo” J. Virol., vol. 70, pp. 894-8960). Deletion of an E2 region of an adenoviral genome decreases the replication and duration of gene expression from the E2-deleted adenoviral vector. It is, therefore, an object of this invention to incorporate into minimal Ad vectors of the present invention DNA sequences derived from the normal cellular genome or equivalent sequences that will facilitate DNA replication of the minimal Ad genome in the target cell. One such sequence that facilitates DNA replication is alphoid DNA. A 16.2 kb sequence of alphoid DNA repeats allows DNA replication but not segregation of the DNA as an artificial chromosome (Calos, M. P. (1996) “The potential of extrachromosomal replicating vectors for gene therapy” Trends in Genetics, vo. 12, pp. 463-466; Krysan, et al. (1993) “Autonomous replication in human cells of multimers of specific human and bacterial DNA sequences” Mol. Cell. Biol., vol. 13, pp. 2688-2696; Haase, S. B. & Calos, M. P. (1991) “Replication control of autonomously replicating human sequences” Nucl. Acids Res., vol. 19, pp. 5053-5058). The present invention provides for the incorporation of the 16.2 kb sequence into the minimal Ad vector. Replication of the minimal Ad vector containing these sequences may extend the persistence of the minimal Ad vector DNA and expression of the gene of interest within the target cell.

Animal model test systems for evaluating the modified vectors of the present invention are also provided. Animal models provided by the present invention include, but are not limited to: 1) transgenic mice comprising the AAVS1 sequence incorporated into their genome for evaluating AAV-based integration mechanisms; and, 2) non-human transgenic animals comprising particular gene sequences operably linked to a developmentally-regulated promoter inserted into their genome.

Those skilled in the relevant art will recognize techniques useful in practicing the present invention. These techniques are described in many generalized references, including, but not limited to, Molecular Cloning: A Laboratory Manual (Sambrook, J. et al., Eds. (1989) Cold Spring Harbor Laboratory Press: Cold Spring Harbor, N.Y.), Gene Expression Technology (Methods in Enzymology, Vol. 185, Goeddel, D., Ed. (1991) Academic Press: San Diego, Calif.), PCR Protocols: A Guide to Methods and Applications (Innis, et al., Eds., (1990) Academic Press: San Diego, Calif.), Culture of Animal Cells: A Manual of Basic Techniques (R. I. Freshney (1987), 2nd edition, Liss, Inc.: New York, N.Y.), Antibodies: A Laboratory Manual (Harlow & Lane (1988) Cold Spring Harbor Laboratory Press: Cold Spring Harbor, N.Y.), Guide to Protein Purification. Methods in Enzymology, vol. 182 (M. P. Deutscher, Ed. Academic Press: San Diego, Calif.; Internet Book of Gene Therapy (1995) Sobol, R. E. & Scanlon, K. J., Eds., Appleton & Lange, Stamford, Conn.; Goldsby, R. A. et al. (2000) Kuby Immunology, 4th ed., W. H. Freeman & Co.; Current protocols in Immunology (1999) Coligan, J., Kruisbeek, A., Margulies, D., Shevach, E., Strober, W., Eds., John Wiley and Sons (most recent update); Benjamini, E. et al. (2000) Immunology: A Short Course, 4th Ed., John Wiley & Sons; Janeway, C., Travers, P., Walport, M. & Shlomehick, M. (2001) Immunobiology, 5th Ed., Garland Publishing; Abbas, A. K., Lichtman, A. H. & Pober, J. S. (2000) Cellular and Molecular Immunology, 4th Ed., W. B. Sanders Co.; Roitt, I. & Delves, P. J. (2001) Roitt's Essential Immunology, 10th Ed., Blackwell Science Inc.; Plotkin, S. A., Orenstein, W. A. & Zorab, R. (1999) Vaccines, 3rd Ed., W. B. Sanders Co.; Thomson, A. (1998) The Cytokine Handbook, 3rd Ed., Academic Press; Teratocarcinomas and embryonic stem cells—a practical approach, Robertson, E. J., Ed. (1987) IRL Press: Washington, D.C.; Gendron-Maguire, M. & Gridley, T. (1993) “Identification of transgenic mice” Methods Enzymol., vol. 225, pp. 794-799; Mann, J. R. (1993) “Surgical techniques in production of transgenic mice” Methods Enzymol., vol. 225, pp. 782-793; Gordon, J. W. (1993) “Production of transgenic mice” Methods Enzymol., vol. 225, pp. 747-771; Mann, J. R. & McMahon, A. P. (1993) “Factors influencing frequency production of transgenic mice” Methods Enzymol., vol. 771-781; Bradley, A. (1987) “Production and analysis of chimaeric mice” In Teratocarcinomas and embryonic stem cells—a practical approach, Robertson, E. J., Ed., pp. 113-151, IRL Press: Washington, D.C.; and the like; as well as references in the foregoing). Other suitable references beyond those listed here would also be known to the skilled artisan and available. All cited references are hereby incorporated by reference.

Within this application, a “transcriptional regulatory region or transcriptional control region” is defined as any nucleic acid element involved in regulating transcription of a gene, including but not limited to promoters, enhancers, silencers and repressors. A “DNA fragment” is defined as a segment of a single- or double-stranded DNA derived from any source. A “DNA construct” is defined as a plasmid, virus, autonomously replicating sequence, phage or linear segment of a single- or double-stranded DNA or RNA derived from any source.

An “expression cassette” is a DNA fragment comprising a coding sequence for a reporter or effector gene operably linked to a transcriptional regulatory region or a transcriptional control region sufficient for expression of the encoded protein in an appropriate cell type. A “reporter construct” is defined as a subchromosomal and purified DNA molecule comprising a gene encoding an assayable product. An “assayable product” includes any product encoded by a gene that is detectable using an assay. Furthermore, the detection and quantitation of the assayable product is anticipated to be directly proportional to the level of expression of the gene. An “effector gene” is defined as any gene that, upon expression of the polypeptide encoded by the gene, confers an effect on an organism, tissue or cell. A “transgene” is defined as a gene that has been inserted into the genome of an organism other than that normally present in the genome of the organism. “Stable gene expression” is defined as gene expression that may be consistently detected in a host for at least a period of time greater than seven days. A gene expressed in a “tissue-specific manner” is that which demonstrates a greater amount of expression in one tissue as opposed to one or more second tissues in an organism.

A “recombinant adenoviral vector” is defined as a adenovirus having at least one segment of heterologous DNA included in its genome. “Adenoviral particle” is defined as an infectious adenovirus, including both wild type or recombinant. The adenovirus may include, but is not limited to, a DNA molecule encapsidated by a protein coat encoded within an adenoviral genome. A “recombinant adenoviral particle” is defined as an infectious adenovirus having at least one portion of its genome derived from at least one other source, including both adenoviral genetic material as well as genetic material other than adenoviral genetic material. “Heterologous DNA” is defined as DNA introduced into an adenoviral construct that was isolated from a source other than an adenoviral genome.

A “treatable condition” is defined as a condition of an organism that may be altered by administration of a form of treatment including but not limited to those treatments commonly defined as being of medicinal origin. A “genetic condition” is defined as a condition of an organism that is a at least partially the result of expression of at least one specific gene including but not limited to the wild-type form of that gene and any mutant form of that gene.

An “antigen” is defined as any molecule capable of inducing an immune response. An “immunomodulatory gene” is defined as any gene that, upon expression of its nucleic acid or protein product, serves to alter an immune response, including both activation and repression or suppression of an immune response. A “tumor suppressor gene” is defined as a gene that, upon expression of its protein product, serves to suppress the development of a tumor including but not limited to growth suppression or induction of cell death. A “growth suppressor gene” is defined as a gene that, upon expression of its protein product, serves to suppress the growth of a cell. An “oncogene” is defined as a cancer-causing gene. A “ribozyme” is defined as an RNA molecule that has the ability to degrade other nucleic acid molecules.

### Basic Concepts of Minimal Ad Vector Systems

1. Composition of the system: The minimal Ad vector system consists of three major parts: 1) a packaging-attenuated helper Ad; 2) a cognate Ad vector having a minimal amount of the viral genome; and, 3) an Ad helper cell line that provide functions of E1 trans-activation like 293 cells and/or regulation of packaging signal for the helper Ad. The packaging-attenuated helper Ad comprises the viral genetic material required for self-replication as well as trans-complementation of minimal Ad vector replication. The helper Ad retains wild-type Ad genetic material except for an E1 deletion or substitution and a manipulated packaging signal useful in controlling or discriminating against packaging of the helper Ad in favor of packaging a minimal Ad vector of the present invention. The minimal Ad vector comprises minimal Ad genetic material including only the inverted terminal repeats (ITRs) and a wild-type packaging signal as cis-elements that serve to promote replication and packaging of the minimal Ad vector. The remainder of the minimal Ad vector comprises transgene or heterologous DNA. The Ad helper cell lines of the present invention are similar to A549 cells and the like in that the cell lines comprise Ad E1 genes and provide Ad E1 gene products that support replication of the helper Ad. The cell lines may further comprise a control mechanism for attenuating packaging of the helper Ad.

2. Mechanism of operation of the system: The packaging protein of Ad is a trans-acting factor present in low amounts in an infected cell and serves as the rate-limiting factor in the packaging of Ad. As the wild-type packaging signal, possessed by the minimal Ad vector of the present invention, is recognized by the packaging protein with higher affinity than the manipulated packaging signal of the helper Ad, packaging of the helper Ad genetic material is partially or completely suppressed in the presence of the minimal Ad vector. This results in preferential packaging of the minimal Ad vector. In order to replicate and package the minimal Ad vector at high-titer, the proteins for viral DNA replication and those for capsid assembly must be provided in adequate amounts. The proteins may be provided from several different sources, including but not limited to a plasmid, a cell line, or a virus. In a preferred embodiment of the present invention, the proteins are provided by the helper Ad. The present invention allows for the helper Ad to remain fully functional in replicating itself within a helper cell such that large quantities of Ad structural proteins are available to the minimal Ad vector. In the absence of the minimal Ad vector, and without selection pressure of the packaging attenuation, the helper Ad is packaged, albeit slowly or ineffectively. Viral DNA replication proteins are also required to amplify the minimal Ad vector DNA for generation of multiple copies of the minimal Ad vector. The replication proteins may be provided from any of several different sources, including but not limited to a plasmid, a cell line, or a virus. In a preferred embodiment of the present invention, the proteins are provided by the helper Ad. The minimal Ad vector, comprising the wild-type packaging signal, is packaged into Ad virions as infective, replication-competent Ad particles. In contrast, helper Ad DNA is competed off by poor recognition or low affinity of the packaging protein for the manipulated packaging signal, and thus remains completely or partially free within the helper cells. By attenuating packaging of the helper Ad and selecting for packaging of the minimal Ad vector, this system of the present invention results in preferential propagation of the minimal Ad vector. The minimal Ad vector produced using this system may be contaminated by low amounts of helper Ad, thus the minimal Ad particle preparation may not be 100% pure. If necessary, the contaminating helper Ad may be removed using biological, biochemical, or physical methods including but not limited to ultracentrifugation through a CsCl gradient.

3. Capability of the system: Three major features of the minimal Ad vector system of the present invention make it unique, sophisticated, and significantly advanced over Ad vectors that are currently available to one skilled in the art. These features include but are not limited to the following: 1) the minimal Ad vector exhibits minimal immunogenicity; 2) the minimal Ad vector is virtually incapable of generating replication competent adenovirus (RCA); and, 3) the minimal Ad vector may comprise much larger segments of heterologous DNA than conventional Ad vectors. Reduced immunogenecity and RCA generation (a major safety concern in the field of gene therapy) is possible because the minimal Ad vectors carry only a minimal amount of viral cis-element (ITRs and packaging signal), and as such, do not encode Ad proteins. A major source of immunogenicity and cytotoxicity of the currently available Ad vectors has thus largely been removed. The cytotoxic, inflammatory, and immunogenic responses normally resulting from expression of Ad viral proteins within a host cell or upon its cell surface are thus reduced.

The minimal Ad vector of the present invention further provides increased capacity for heterologous DNA than convention Ad vectors. Wild-type Ad has an average genome size of 36 kb. The maximal packaging capacity of Ad is roughly 105% of the genome, i.e. approximately 38 kb. The minimal Ad vector of the present invention may comprise less than 1 kb of Ad genetic material; therefore, the capacity of the minimal Ad vector for heterologous DNA may be about 36 kb to about 37 kb. The heterologous DNA may include but is not limited to a transgene expression cassette, a regulatory element, or a transcriptional control region operatively linked to a reporter or effector gene. The expression cassette may include but is not limited to single or multiple expression cassettes. The regulatory element may include but is not limited to a DNA sequence for controlling transgene retention, integration, transcription, and/or vector targeting.

### Packaging-Attenuated Helper Adenoviruses

a. A prototype structure of the helper: The helper Ad vector comprises a wild-type Ad genome having a manipulated packaging signal and an altered E1 gene. For safety reasons, the helper Ad must be defective in replication, such as the currently available E1-deleted or substituted viral constructs. For the purpose of controlling packaging in the presence of the minimal Ad vector, the helper must be also defective in packaging (detailed below). Therefore, the general structure of the helper can be summarized as an Ad vector having a wild-type genome except that the E1 region and packaging signal are manipulated. However, the other essential regulatory genes of Ad such as E2 and E4 may also be manipulated. The viral genome may be split into fragments in order to further disable the replication competence of the helper Ad or to reduce the genome size of the helper Ad in order to separate it from the minimal Ad vector using a biological, biochemical, or physical method including but not limited to ultracentrifugation through a CsCl gradient. As long as the titer of the helper Ad is not significantly affected, both a defect in viral replication and attenuation in packaging of the helper Ad may be included in the design of the helper Ad.

b. The general function of the helper Ad: The primary function of the helper Ad is to supply the capsid proteins required to package the minimal Ad vector. In order to provide the proteins, the helper Ad must be able to replicate within the host cell, although less efficiently than wild-type Ad. Preferably, DNA replication and transcription of the helper genome is not affected. If synthesis of the helper Ad genome were inhibited, the yield of the late gene products (the capsid proteins) would be altered and may adversely affect the titer of the minimal Ad vector (i.e., the titer will be reduced). For certain applications, removal of the helper Ad from the minimal Ad may not be necessary. In such situations, the stringency of packaging attenuation of the helper Ad may be greatly reduced.

c. Designs for packaging attenuation: The purpose for attenuation of packaging the helper Ad is to reduce the potential for helper Ad contamination in preparations of the minimal Ad vector. This is especially important when a relatively pure batch of the minimal Ad vector is required for a particular application. The packaging function of the helper Ad is designed to be defective but not completely disabled, because the helper Ad must be able to propagate, albeit slowly, in the absence of a minimal Ad vector. The following genetic manipulations may be utilized to generate a packaging-attenuated helper Ad.

1. Packaging signal mutation: The Ad5 packaging signal is composed of a repeated element that is functionally redundant (18). Partial deletions of the packaging signal elements have been shown to reduce the yield of mutant Ad from several fold to approximately a hundred fold as compared to that of Ad having a wild-type packaging signal (18). The design of the packaging signal mutation of the present invention may therefore incorporate a partial deletion of the consensus adenosine-enriched motif (e.g., “A-repeat”: TAAATTTG) from the wild-type Ad packaging signal.

2. Synthetic packaging signal: Since the Ad5 packaging signal has a consensus A (adenosine) enriched motif (e.g. A-repeat: TAAATTTG), incorporation of an array of tandem repeats including but not limited to a selected A-repeat or any synthetic DNA motifs that may alter the affinity of the packaging protein for the artificial packaging signal.

3. Packaging signal interference: The Ad packaging signal is a specific DNA sequence that is recognized and bound by the packaging proteins. In order to interfere with the effective binding of the packaging proteins to the signal, other DNA sequences may be placed in proximity to or within the A-repeat array of the helper Ad packaging signal. The inserted DNA sequences allow binding by their cognate DNA binding proteins that may positionally compete off the binding of the Ad packaging proteins to the Ad packaging signal.

4. Packaging signal relocation: The wild-type Ad packaging signal is positioned at the left end of the wild-type Ad genome. Investigators have found that the packaging signal may be located at the right end and retain its function (75) indicating that the packaging signal may be relocated. Positioning the manipulated packaging signal in a location other than wild-type may be useful to further attenuate the packaging efficiency of the helper Ad. In addition, relocation of the packaging signal to another region of the Ad genome may be helpful in minimizing the possibility of reversion of the helper Ad back to wild-type Ad through homologous recombination between the engineered packaging signal of the helper Ad and the wild-type packaging signal of the minimal Ad vectors (i.e., generation of RCA).

5. Further possibilities: To attenuate packaging of the helper Ad to minimize the contamination of the helper to a preparation of the minimal Ad vectors, two factors may be considered: cis-elements and trans-acting factors. Therefore, other possible designs may be oriented towards manipulation of either or both of these two factors. An example of cis-elements that may be manipulated is the A-repeat motif. An example of a trans-acting factor that may be manipulated is a packaging protein. Further consideration should be a controllable mechanism of packaging without sacrificing the high titer output of the minimal Ad vectors by the system.

### Minimal Ad Vectors

a. The basic structure of the minimal Ad vector: Ad vectors may be utilized as circularized plasmids by fusion of the Ad ITRs (54). The simplest plasmid form of the minimal Ad vector of the present invention is a circular DNA molecule comprising an ITR fusion sequence (comprising an Ad ITR having a wild-type packaging signal), a plasmid DNA replication origin, and a polycloning site consisting of one or multiple restriction enzyme sites. The ITR fusion sequence includes the left end of the wild-type Ad, preferably from map unit 0 to 1, and the right end, preferably from map unit 99 to 100. An Ad DNA replication origin is located in each ITR and the wild-type packaging signal is located adjacent to the left ITR.

b. The structural and functional possibilities of the minimal Ad vectors: Other DNA sequences and elements including, but not limited to, those listed below may be included in a minimal Ad vector:

1. Expression cassettes of transgenes: An expression cassette is a basic transcription unit. A simple expression cassette of a given gene generally comprises a transcriptional control region, a gene of interest (i.e., heterologous DNA, insert DNA), and a polyadenylation (polyA) signal. Within an expression cassette, two or more genes may be included as bi- or polycistronic units, as long as additional elements for translation or splicing of RNA are provided between the genes. Generally, minimal Ad vectors comprise one or multiple expression cassettes.

2. Functional elements for vector DNA retention: Elements that may assist in integration of the expression cassette into target cell genome (i.e., AAV integration elements) or maintain the minimal Ad vector as an episomal form in a host cell. Elements that have been shown to assist in integration are the inverted terminal repeats (ITRs) and the Rep78/68 proteins of the adeno-associated virus (AAV). AAV utilizes these elements to achieve specific integration of its genome in human chromosome 19 (19q13.3-qter) at a site named AAVS1.

Although AAV has been considered as a candidate vector for gene therapy, several limitations have been identified by investigators. AAV is limited by: 1) low capacity for exogenous DNA (4.3 kb); 2) difficulty in achieving high titers in large-scale preparations; and, 3) loss of specific integration of the recombinant AAV. Each of these have proven to be difficult challenges to those skilled in the art. The present invention combines the advantages of the minimal Ad vector with the integration capacity of AAV by incorporating the AAV-ITR sequences and Rep 78/68 expression cassette (Rep expression cassette) into the vector.

Mechanisms that may also be included in the minimal Ad genome include extrachromosomal replication sequences (Calos, M. P. (1996) “The potential of extrachromosomal replicating vectors for gene therapy” Trends Genet., vol. 12, pp. 463-466). Such sequences, comprised of either chromosomal or viral sequences, serve to enable the vector to efficiently replicate and be retained within a mammalian cell. The sequences may include a replication component such as human genomic DNA and/or a retention component such as human centromere sequence or sequence derived from the Epstein-Barr virus (EBV) such as the oriP family of repeats and/or EBNA-1 (Calos, M. P. (1996) supra). The human human genomic DNA may comprise a telomere and/or alphoid DNA (Calos, M. P. (1996) supra). By including such elements in a minimal Ad vector, the minimal Ad genome will replicate to a higher copy number in the host cell, thus increasing the probability that the minimal Ad genome will be packaged at a greater effiency than that helper virus. Additionally, these sequences serve to lengthen the duration of expression of the effector or reporter gene within the host cell. Such functions would be useful in utilization of the minimal Ad vector for gene therapy.

3. Regulatory elements for control of DNA transcription: Elements having transcriptional regulatory function including but not limited to enhancers, repressors, activator-binding sites, introns, and 5′ or 3′-untranslated regions. Various combinations of such elements may be incorporated into the minimal Ad to enhance or control expression of a gene of interest.

4. Elements for vector and transgene targeting: Targeting can be achieved by several methods including but not limited to vector surface modification and tissue-specific expression. Tissue specific promoters may be utilized to drive gene expression in a specific cell type or tissue. Many such promoters are available to one of skill in the art.

5. Further supporting elements: These may include but are not limited to DNA replication origins for prokaryotic or eukaryotic cells, plasmid or vector selection markers, and vectors backbones. The skilled artisan would understand the need to incorporate one or more of such supporting elements into the minimal Ad vector as necessary.

c. Designs for high titer production of the minimal Ad vectors: High-titer production of the minimal Ad vectors is another major aspect of this invention. One advantage of Ad vectors over other viral vectors is that Ad particles are conducive to preparation of high-titer preparation stocks (Hitt, et al. In Methods in Molecular Genetics, Adolph, K. W., Ed., vol. 7, pp. 13-30, Academic Press: San Diego, Calif.). High-titer propagation of Ad is possible due mainly to the large quantity of viral capsid proteins and viral genome copies produced within a host cell such as a 293 cell during infection. Listed below are some of the factors that may be considered in designing methods for generating high-titer minimal Ad vectors:

1. Enhanced DNA replication: Ad has its own enzymatic system for DNA replication. The E2 region proteins are the major trans-acting elements responsible for viral DNA replication. The replication origins are the cis-elements located at the either or both ends of the viral genome. To support minimal Ad genome replication, a sufficient quantity of E2 proteins must be provided by the helper virus. High-level expression of E2 proteins (encoded within the E2 region of Ad) is ensured by proper design of the helper virus genome. Other such mechanisms for increase in copy numbers of the minimal Ad genome may also be considered. Such mechanisms may include but are not limited to insertion of the the SV40 origin of DNA replication (McGrory, et al. (1988) “A simple technique for rescue of early region I mutations into infectious human adenovirus type 5” Virol., vol. 163, pp. 614-617) into the minimal Ad genome to increase the copy numbers of the minimal Ad, concomitant with SV40 T-Ag expression in the helper cell.

2. Enhanced packaging signal: A higher number or more efficient packaging sequences may be utilized by, for example, incorporating a greater number of tandem repeats at one or both ends of the minimal Ad genome, or by incorporation of one or multiple synthetic packaging signals that function in a more efficient manner than the wild-type packaging signal.

3. Enhanced packaging process: The packaging process and mechanism of Ad are not yet fully understood by those skilled in the art. Whether DNA binding proteins other than the packaging signal of Ad have synergistic roles for packaging is not certain. The sequences for which a DNA-binding protein shows affinity, referred to as “anchorage points for packaging” and naturally existing within the Ad genome, may be incorporated into the minimal Ad vector.

### Ad Helper Cell Lines

1. Basic elements and general functions of Ad helper cells: The cell line of the present invention (that serves as the host cell) provides several important modifications that improve upon conventionally utilized cell lines, (e.g., A549 cells, 293 cells (ATCC# CRL1573) and the like). In a preferred embodiment, the host cell comprises a nucleic acid sequence encoding an Ad-E1 fragment for trans-activation of the transcription program of the helper Ad genome. Unique from the E1 fragment of 293 cells currently available to one skilled in the art, a cell line of present invention may comprise nucleic acid sequence encoding the E1 fragment having no overlapping nucleic acid sequence with the helper Ad genome. The present invention, therefore, eliminates one of the current difficulties associated with Ad vectors: generation of wild-type Ad or replication-competent Ad (RCA) through homologous recombination. Other elements may include, but are not limited to, genes involved in the support of high copy-number production of the minimal Ad vector, enhancing packaging of the minimal Ad vector, and/or attenuating the packaging of the helper Ad.

2. Assistance mechanisms for packaging attenuation of the helper Ad: Other methods by which packaging of the helper Ad is attenuated may include interference with the binding site for the packaging protein by placement of a binding site for a different protein nearby the packaging protein binding site within the helper Ad genome. Such a system may include but is not limited to utilization of the tetracycline-repressor (Tet-R), a recombinase, and/or an altered packaging protein. In a preferred embodiment, the different protein is expressed within a host cell. Tet-R may bind to a manipulated packaging signal of a helper virus comprising a binding site for Tet-R, the tet-operon (Tet-O), and thereby repress packaging by inhibiting binding of the packaging protein. Binding of Tet-R to Tet-O is controlled by tetracycline. Addition of tetracycline into the cell culture medium results in binding of tetracycline to the Tet-R and prevents it from binding tet-O. Removal of the tetracycline frees Tet-R for binding to the engineered packaging signal and serves to further attenuate packaging of the helper virus.

Expression of a recombinase such as Cre or Flp may also inhibit packaging provided the packaging signal of the helper virus is flanked by a recombination site, such as lox-p or FRP, respectively (Parks, et al. (1996) “A helper-dependent adenovirus helper system: Removal of helper virus by cre-mediated excision of the viral packaging signal” Proc. Natl. Acad. Sci. USA, vol. 93, pp. 13565-13570; Broach, et al. (1982) “Recombination within the yeast plasmid 2 mu circle is site-specific” Cell, vol. 29, pp. 227-234). Other genetic modifications within the helper virus genome may also be provided separately or in addition to those listed above to further attenuate helper virus replication.

The packaging protein may be altered by any of several methods including but not limited to utilization of a specific serotype or species difference in the packaging signal to differentiate packaging of the minimal Ad from the helper Ad provided the specific packaging protein of Ad is identified. Additionally, the packaging protein may be altered by genetic modification of the gene encoding the packaging protein. The modification may alter the packaging protein such that its binding preference for the wild-type packaging signal is increased. The modified packaging protein, then, may further provide preferred packaging of the minimal Ad genome.

3. Assistance mechanisms for high-titer production of the minimal Ad vectors: Modifications of the minimal Ad vector designed to increase the copy number of the minimal Ad genome within a host cell are useful in the development of a high-titer minimal Ad vectors. Expression of the SV40 T-Ag (mutated T-Ag with no transforming activity) by the host cell may increase the copy number of the minimal Ad genome, provided a SV40 DNA replication origin is incorporated into the minimal Ad plasmid vector.

### Potential Applications of Minimal Ad Vectors of the Present Invention

a. Delivery of genes for therapy of genetic diseases in vivo: Large capacity for exogenous nucleic acid is necessary for delivery of a large therapeutic gene or multiple genes as well as for transfer of regulatory elements and/or other related genes along with the primary therapeutic genes that will determine controllable or tissue-specific expression and may result in a more effective therapeutic effect. An example includes but is not limited to cystic fibrosis in which controllable expression of several genes is required to optimize cystic fibrosis gene therapy. Gene therapy of Duchenne muscular dystrophy (DMD) is another example of a condition for which treatment would require a large capacity vector. For treatment of this disease, genes including but not limited to muscle and nerve growth factors may be required to be co-delivered in order to generate a complete physiological effect to restore the muscle function of the patients.

b. Induction of host anti-cancer immunity through intratumoral injection of the vectors: Ad vectors demonstrate high levels of infectivity in cultured tumor cells and different types of solid tumor models in vivo. This characteristic of the Ad vector has been utilized in the treatment of cancer. The efficacy of treatment depends upon the genes that are delivered by the vectors. Multiple genes including but not limited to those having combined functions of tumor suppression and immunomodulation are utilized to optimize the anti-cancer effect. The minimal Ad vector has the capacity to deliver multiple genes and is useful in constructing anti-cancer Ad vectors for intratumoral injection.

c. Modulation of host immunity by genetic modification of the graft cells or tissues: Transplantation requires transient or permanent suppression of the host immunity. To deliver immune suppression genes into cells or tissues including, but not limited to, graft cells or graft tissues may be an alternative approach to the administration of immunosuppressive agents. Examples of genes encoding immune suppression proteins useful in the present invention may include, but are not limited to, TGF-B, IL-10, viral proteins HSV-ICP47 and CMV-US 11, and secretable Fas-ligand proteins that may be delivered alone or in combination by the minimal Ad vectors of the present invention.

d. Modification of target cell function or regulation target cell growth in vivo by genetic modification: Ad vectors have a distinct advantage over other viral vectors in that production of high titer stocks is possible, which is useful for in vivo gene therapy. Because the minimal Ad vectors contain only minimal amounts of cis-elements of the Ad genome, the immunogenicity of minimal Ad is minimized. Therefore, the minimal Ad vector will be useful for modifying target cell function or regulating target cell growth in vivo by genetic modification.

e. Specific delivery of transgenes to target cells or tissues in vivo by surface modification of the vectors: The genes encoding the adenoviral hexon and fiber proteins are engineered to fuse with certain epitopes or ligands (e.g., the protein A that binds to Fc fragment of IgG) present on the target cell surface. These modified genes are incorporated into the recombinant viral genome for generation of the viruses having surface sites that interact with ligands that function as targeting agents on the target cell surface. The viral particles thus produced have tissue or cell recognition capabilities.

f. To be used for Ad-mediated vaccination via direct in vivo approaches: For the purpose of vaccination, the immunogenicity of the E1-substituted Ad vectors may provide benefits, and has been used in development of Ad-based recombinant vaccines. Minimal Ad vectors utilized in this type of application use the helper virus including but not limited to E1-substituted Ad vectors as well as co-delivery of genes encoding antigens and immunogens that provide immunization.

In addition, it is also possible to construct minimal Ad vectors comprising large portions of the genome of an infectious agent (e.g., HIV, HPV, and the like) that will direct expression of most proteins expressed by the infectious agent without replication or growth of the agent. For example, it is possible to incorporate large regions of the HIV genome into a minimal Ad such that replication of HIV is not possible. Similarly, one could incorporate regions of other viruses such as Human Papilloma Virus (HPV) into the minimal Ad to generate immunity against HPV. It may also be possible to incorporate nucleic acid encoding immunogens from multiple pathogens into a single vector. For instance, it is possible to incorporate HIV antigens and HPV antigens into a single minimal Ad, thus providing a method for immunizing a host against both pathogens using a single vector. Further, one could incorporate an expression cassette encoding an immunomodulating protein such as a cytokine (e.g., GM-CSF) in order to enhance the immune response. One of skill in the art would understand the various combinations of such immunogens and/or immunomodulating proteins may be incorporated into a single minimal Ad for administration to a host.

g. To be used for ex vivo gene delivery: Transient gene retention and expression associated with the use of conventional Ad vectors has prevented Ad from being widely used in ex vivo gene delivery protocols. The minimal Ad vectors, having DNA retention mechanisms, are useful for this purpose. Also, the high infectivity of Ad in cultured cell lines make the minimal Ad vectors very effective gene delivery system for ex vivo approaches toward gene therapy.

h. To be used as tools for basic research and development of adenovirology and novel vector construction: The minimal Ad vector system itself has a great value for basic adenovirology studies. The construction and demonstration of the feasibility and operation are already a breakthrough in the field. The helper Ad and the minimal Ad provide convenient tools for study of the Ad and its potential applications. This is particularly true for the minimal Ad vector. The characterization of the replication, packaging, and propagation efficiency of the minimal Ad will provide the field with important new information, which was previously unavailable.

i. To be used in combination with other methodology in the field of gene transfer and therapy: Ad vectors have been used together with polylysine, liposome, and other conjugation materials as a gene delivery complex. The minimal Ad vectors can also be used with these compounds as well as any other compound that comprise the ability to serve as a gene delivery complex.

j. To be used for other purposes in the field of gene transfer and therapy: The minimal Ad vector system has a great potential to be used for gene transfer and therapy in addition to what have been discussed above. Such possibilities will arise with further developments in the field of genetic medicine, including, but not limited to, genetic vaccination, gene therapy, gene transer, and/or gene delivery.

Non-limiting examples of diseases and genes that may be treated or utilized, respectively, using minimal adenoviral vectors of the invention are summarized in Table 1.

TABLE 1*

| Disease | Frequency | Defect protein/gene | Malfunction manifestation | Current treatment |
|---|---|---|---|---|
| Apo A-I structural | 1:1,000 | Apo A-I | In a few cases, causes decreased | Dietary treatment; HMG-CoA |
| mutations | | | HDL-C levels with no increase in | reductase inhibitors. |
| | | | coronary heart disease. Two | |
| | | | mutations lead to amyloidosis. | |
| Familial ligand- | ˜1:500-1,000 | Apo B-100 | Impairment of receptor-dependent | Dietary treatment; HMG-CoA |
| defective apo B-100 | heterozygous | | endocytosis of LDL in all | reductase inhibitors. |
| | | | nucleated cells. Elevated levels of | |
| | | | LDL in plasma. Increased risk of | |
| | | | atherosclerotic heart disease. | |
| Familial | ˜1:3,000 | Apolipoprotein B-100 | Failure to secrete VLDL and | For homozygous: Restriction |
| hypobetalipoproteinemia | heterozygous | | chylomicrons if truncations involve | of dietary fat and |
| | | | more than two-thirds of apo B-100 | supplementation of Vitamin E. |
| | | | sequence. Less severe truncations | |
| | | | do not prevent secretion but result | For heterozygous: Moderate |
| | | | in abnormal plasma lipoproteins. | doses (400-800 mg/day) of |
| | | | Heterozygotes show lipid | tocopherol. |
| | | | abnormalities but are usually | |
| | | | asymptomatic. Homozygotes may | |
| | | | have a syndrome similar to | |
| | | | homozygous abetalipoprotinemia. | |
| Familial type III | 1:1,000-5,000 | Apolipoprotein E | Accumulation in plasma of | Dietary treatment; HMG-CoA |
| hyperlipoproteinemia | | | chylomicron and VLDL remnants | reductase inhibitors; nicotinic |
| (dysbetalipoproteinemia) | | | (collectively, β-VLDL), leading to | acid plus fibric acid derivatives. |
| | | | hyperlipidemia and atherosclerosis. | |
| Familial | 1:500 in most | LDL receptor | Absent or deficient receptor- | Dietary treatment; HMG-CoA |
| hypercholesterolemia | populations | | mediated endocytosis of LDL | reductase inhibitors; nicotinic |
| | | | causes LDL to accumulate in | acid plus bile acid-binding |
| | | | plasma. Hyper-cholesterolemia and | resins. For homozygous, may |
| | | | atherosclerosis result. | need probucol, portacaval |
| | | | | anastomosis, plasma exchange, |
| | | | | and liver transplantation. |
| von Willebrand disease | 1:8,000 | von Willebrand factor | Abnormal platelet adhesion and | Vasopressin analogue DDAVP |
| | | | mildly to moderately reduced | for mild deficiency. Plasma |
| | | | factor VIII levels cause bleeding. | infusion and vWF treatment for |
| | | | | severe patients. |
| Factor VIII deficiency | 1:10,000 males | Factor VIII | Factor VIII fails to function as a | Prophylaxis, plasma infusion, |
| (hemophilia A) | | | cofactor for activation of factor X | and Factor VIII treatment. |
| | | | and impairs clotting cascade. | |
| Factor IX deficiency | 1:70,000 | Faxtor IX | Impaired blood coagulation. | Prophylaxis, plasma infusion, |
| (hemophilia B) | | | | and Factor IX treatment. |
| Factor XI deficiency | ˜1:1,000 in | Faxtor XI | Deficiency of protein leads to | Prophylaxis, plasma infusion, |
| (hemophilia C) | Ashkenazi Jews of | | impaired contact activation and | and Factor XI treatment. |
| | Israel | | mild bleeding tendency. | |
| Antithrombin | ˜1:5,000 | Antithrombin | Impaired inhibition of coagulation | Long-term anticoagulation |
| deficiency | | | factors IIa, IXa, and Xa in plasma | therapy, plasma or |
| | | | causes recurrent venous | antithrombin infusion. |
| | | | thrombosis. | |
| Protein C deficiency | 1:10,000 | protein C | Impaired regulation of blood | Long-term anticoagulation |
| | | | coagulation. Predisposition to | therapy, plasma or protein C |
| | | | thrombosis. | infusion. |
| α₁-Antitrypsin | 1:7,000 northern | α₁-Antitrypsin | Liver storage of polypeptide; | α₁-Antitrypsin replacement |
| deficiency (Z variant) | Europeans: 1:3,000 | | plasma deficiency of protein | therapy. |
| | Scandinavians | | allows overactivity of elastase. | |
| C2 deficiency | ˜1:10,000 | Complement component 2 | Markedly reduced activation of the | Replacement therapy. |
| | | | classic pathway. | |
| Phenylketonuria (PKU) | ˜1:10,000 births | Phenylalanine | Hepatic enzyme deficiency causes | Low-phenylalanine-diet therapy |
| due to PAH deficiency | (considerable | hydroxylase (PAH) | hyperphenylalaninemia; plasma | and enzyme (bacterail |
| | regional variation) | | values persistently above 1 mM | phenlalanine ammonia lyase) |
| | | | associated with impaired cognitive | treatment. |
| | | | development. Risk of maternal | |
| | | | hyperphenyl-alaninemia effect on | |
| | | | fetus carried by female proband. | |
| Transferase deficiency | 1:35,000-60,000 | Galactose 1-phosphate | Accumulation of galactose, | Elimination of dietary lactose; |
| galactosemia | | uridyltransferase | galactitol, galactose 1-phosphate, | avoidance of galactose. |
| | | | and galactonate causes cataracts, | |
| | | | mental retardation, and liver and | |
| | | | kidney dysfunction. | |
| Hereditary fructose | 1:20,000/Switzerland | Fructose 1,6-bisphosphate | Ingestion of fructose causes the | Elimination from the diet of all |
| intolerance | | aldolase B | accumulation of fructose 1- | sources of sucrose and fructose, |
| | | | phosphate and hence multiple | with supplement of vitamin C. |
| | | | dysfunctions in small intesting, | |
| | | | liver, and kidney. | |
| Glycogen storage disease | ˜1:100,000 | Glucose 6-phosphatase | Hypoglycemia, hyperlipidemia, | Dietary restriction; nocturnal |
| type Ia (von Gierke | | | hyperuricemia, and hyperlactic | nasogastric infusion in early |
| disease) | | | acidemia. Glycogen accumulation | infancy; portacaval shunts, liver |
| | | | in liver and kidney. | transplantation. |
| Glycogen storage disease | ˜1:125,000 | Amylo-1, 6-glucosidase | A glycogen with shorter outer | Dietary restriction. |
| type III | | (debrancher enzyme) | chains (limit dextrin) in liver | |
| | | | and/or muscle. Moderate | |
| | | | hypoglycemia and hyperlipidemia. | |
| | | | Muscle weakness mostly in adults. | |
| Menkes disease | 1:250,000 | P-type ATPase (Cu²⁺) | Defective intracellular transport of | No specific or effective |
| | | transport | copper leads to deficiency of | treatment. Presymptomatic |
| | | | copper-containing enzymes and | treatment with copper |
| | | | causes arterial and brain | histidinate can modify the |
| | | | degeneration | disease substantially. |
| Wilson disease | 1:50,000 | P-type ATPase | Defective biliary excretion of | Penicillamine, trientene or |
| | | [membrane cation (Cu²⁺) | copper leads to accumulation in | orraly administered zinc salts. |
| | | transporter] | lever (cirrhosis), cornea (Kayser- | Liver transplantation may be |
| | | | Fleischer rings), and basal ganglia | applied for irreversible liver |
| | | | (movement disorder). | damage. |
| Carbamyl phosphate | 1:70,000-100,000 | Carbamyl phosphate | Impaired urea formation leads to | Dietary restriction, sodium |
| synthetase deficiency | | synthetase I | ammonia intoxication | phenylbutyrate, and citrulline. |
| Ornithine | 1:70,000-100,000 | Ornithine | Impaired urea formation leads to | Dietary restriction, sodium |
| transcarbamylase | | transcarbamylase | ammonia intoxication | phenylbutyrate, and arginine. |
| deficiency | | | | |
| Argininosuccinic acid | 1:70,000-100,000 | Argininosuccinic acid | Impaired urea formation leads | Dietary restriction and arginine. |
| synthetase deficiency | | synthetase | to ammonia intoxication | |
| Methylmalonic acidemia | 1:20,000 | Methyldmalonyl-CoA | Accumulation of methylmalonate | Dietary protein restriction and |
| (2 allelic variants | | mutase (MUT) | leads to metabolic ketoacidosis and | oral antibiotic therapy. |
| designated mut° and | | apoenzyme | developmental retardation | |
| mut-) | | | | |
| Methylmalonic acidemia | 1:20,000 | cblA form: unknown: | Impaired adenosylcobalamin | Cyanocobalamin or |
| (2 nonallelic forms | | cblB form: | syntheses leads to deficient | hydroxocobalamin treatment |
| designated cblA and | | ATP:cob(I)alamin | methylmalonyl-CoA mutase | with dietary protein restriction. |
| cblB) | | adenosyltranferase | (MUT) activity; clinical and | |
| | | | chemical findings resemble those | |
| | | | in apoprotein MUT deficiency | |
| Xanthinuria | 1:45,000 | Xanthine dehydrogenase | Accumulated substrate (xanthine) | No specific or effective therapy |
| | | (xanthine oxidase) | can crystallize in kidney, urinary | is available. Avoidance of |
| | | | tract, or muscle, causing renal | purine-rich foods is advised. |
| | | | failure, nephrolithiases, or | |
| | | | myopathy. Impaired/disturbed drug | |
| | | | metabolism. | |

*Table 1 is composed of the data and information from Ref. No. 55.

Another embodiment of the present invention includes pharmaceutical compositions comprising minimal Ad vectors of the present invention. The pharmaceutical compositions may be made up in a solid form (including granules, powders or suppositories) or in a liquid form (e.g., solutions, suspensions, or emulsions). Solid dosage forms for oral administration may include capsules, tablets, pills, powders, and granules. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents. The compounds of the present invention can be used in the form of salts derived from inorganic or organic acids. Numerous references that further teach pharmaceutical compositions and/or practices are known to those skilled in the art, including, but not limited to: Remington: The Science and Practice of Pharmacy (formerly Remington's Pharmaceutical Sciences), Mack Publishing Co.; Delgado & Remers, Wilson and Grisvold's Textbook of Organic, Medicinal and Pharmaceutical Chemistry, Lippincott-Raven; Bolton, Pharmaceutical Statistics, M. Dekker Publishing; Tietz's Fundamentals of Clinical Chemistry, Burtis, Ed., W. B. Saunders; Stoklosa, Pharmaceutical Calculations, Lippincott Williams and Wilkins; Martin et al., Physical Pharmacy, Lippincott Williams and Wilkins; Martindale—The Complete Drug Reference (formerly Martindale's Extra Pharmacopoeia), The Pharmaceutical Press; Rowland and Tozer, Clinical Pharmacokinetics, Lippincott Williams and Wilkins; Principles of Medical Pharmacology, Kalant & Roschlau, Eds., B.C. Decker, Inc.; Rang et al., Pharmacology, Churchill Livingstone; The Pharmacological Basis of Therapeutics, Goodman & Gilman, Eds., McGraw-Hill; Knevel et al., Quantitative Pharmaceutical Chemistry, Waveland Press, Inc.; Applied Therapeutics, The Clinical Use of Drugs, Young, Ed., Koda-Kimble, Applied Therapeutics, Inc.; Clinical Pharmacy and Therapeutics, Herfindel et al., Eds., Williams and Wilkins; Pharmacotherapy: A Pathophysiologic Approach, DiPiro, J. T. et al., Eds., Elsevier Science Publishing Co., Inc.; U.S.P. Dispensing Information, United States Pharmacopeial Convention, Inc.; Hansten & Horn, Drug Interactions and Updates, Applied Therapeutics; Cipolle et al., Pharmaceutical Care Practice, McGraw-Hill, Health Professionals Division; and the like; as well as references in any of the foregoing).

While the vectors of the invention can be administered as the sole active pharmaceutical composition, they can also be used in combination with one or more vectors of the invention or other agents. When administered as a combination, the therapeutic agents can be formulated as separate compositions that are given at the same time or different times, or the therapeutic agents can be given as a single composition.

The following Examples serve to illustrate particular embodiments of the present invention and should not be construed as limiting the specification and claims in any way. The techniques and methods below are further described in related patent applications (see Related Applications section, supra).

### EXAMPLES

### Example 1

### Construction and Characterization of A Packaging-Signal Mutated Helper Ad and Minimal Ad Vectors That Carry Green Fluorescence Protein (GFP) Reporter Gene

1.1 Generation of a Packaging-Signal Mutated Helper Ad:

Several packaging signal deletion-mutants of Ad5 have been described (90). Mutant dl10/28 (also described as dl309-194/243:274/358) contains a deletion between nt 194 to 243 and between 274 to 358 of Ad5. dl10/28 virus was generated by the method of Stow (89) by ligation of a plasmid containing the left end of Ad5 with this double mutation (pE1A-10/28) and the rest of Ad5 genome (90). dl10/28 showed a 143-fold decrease in virus yield in a single virus infection and, when co-infected with wild type virus, was not detected. It was reasoned that with a helper virus containing the same mutation as dl10/28 it should be possible to amplify the virus, although at low yields, and in the presence of mini-viral vector containing the wild type packaging signal the helper virus should remain unpacked.

The packaging signal was amplified by PCR from pE1A-10/28 using the following primers:

R7: 5′-GGAACACATGTAAGCGACGG (SEQ ID NO: 3)

(nt 137 to 163 of Ad5 with AflIII site underlined)

R8: 5′-CCATCGATAATAATAAAACGCCAACTTTGACCCG (SEQ ID NO: 4)

(nt 449 to 421 with Cla I site attached).

The amplified 133 bp fragment was cut with AflIII and ClaI and used to substitute the corresponding sequence of the shuttle vector GT4004. GT4004 derives from pXCX2 (91) by extending the Ad5 left region from XhoI site (nt 5792, 16 mu) until SnaBI site (nt 10307, 28 mu), therefore GT4004 contains the left end of Ad5 from 0 mu to 1.2 mu with the AflIII site at 0.38 mu, an E1 deletion from 1.2 mu to 9.2 mu with ClaI site in this deletion point and the rest of the left arm of Ad5 until 28 mu. This extended left arm increases the frequency of homologous recombination used to generate recombinant virus. GT4004 with the wild type packaging signal substituted by the deleted one was named as GT5000. The β-gal expression cassette from pTkβ (Clontech, Calif.) was cut as a SalI fragment, blunted with Klenow enzyme and inserted into the blunted ClaI site of GT5000. The resulting plasmid, GT5001, contains therefore the double-deleted packaging signal and the E1 region of Ad5 replaced by the β-gal gene driven by the Tk promoter. This construct allows for detection of helper virus by X-gal staining.

To generate the helper virus the method described by Graham and Prevec was used (91). An early passage of 293 cells obtained from ATCC, were grown in MEM-10% Horse Serum and seeded in 60 mm plates. At 30% confluence cells were cotransfected by CaPO₄ using 2 mg of GT5001 and 4 mg of pJM17 (91) per plate. Three days after cotransfection cells were overlaid with medium containing 0.5% agarose and thereafter the medium above the overlay was changed every-other day. When plaques became visible, X-gal (40 mg/ml in DMSO) was directly added to the medium to 100 mg/ml an let incubating overnight. Plaques producing the desired helper virus were identified by the blue color. Blue plaques were fished and the agarose plugs were resuspended in 1 ml MEM-10% FBS. 370 ml of this medium were processed for PCR amplification of the packaging signal as follows: 40 ml of 10×DNase I buffer (400 mM Tris-HCl pH 7.5, 60 mM Cl₂Mg, 20 mM Cl₂Ca) (as a control of the treatment a tube with 0.5 mg of the shuttle vector was used) and 1 ml of DNase I (Boehringer M, 10 u/ml) were added and incubated for 1 h at 37° C. DNase I was inactivated and viral capsids were opened by adding: 32 ml EDTA (0.25 M), EGTA (0.25 M), 10 ml SDS (20%), 5 ml Proteinase K (16 mg/ml) and incubating at 56° C. for 2 h. After one phenol:chlorophorm:isoamyl alcohol (1:1:1/24) extraction, 1 ml of yeast tRNA (10 mg/ml) was added to help precipitation of viral DNA which was collected by centrifugation at 12000 rpm in a microcentrifuge and resuspended in 20 ml of H₂O. 5 ml were used for a PCR reaction with primers R7 and R8.

One blue plaque with the desired deleted packaging signal was further amplified in 293 cells grown in DMEM-10% FBS. Hereafter named AdHelper-βgal or AdHβ. The virus was extracted at 48 h post-infection by centrifugation of the collected cells at 800 g for 5 min and three cycles of quick freeze and thaw of the cell pellet. This crude extract from X cells was used to infect 3X cells (amplification scale was 1 to 3 in contrast to 1 to 20 for a virus with wild type packaging signal) and plaques identified by staining with X-gal. At every passage the deleted size of the packaging signal was verified by PCR of the supernatant. This deletion and the β-gal expression were stable in all the passages analyzed. At passage 9 AdHβ was purified by CsCl. Purification was done by three cycles of freeze-thawing, layering the lysate onto a step gradient of 0.5 ml CsCl 1.5 mg/ml+2.5 ml CsCl 1.35 mg/ml+2.5 ml CsCl 1.25 mg/ml, and centrifuging in a SW41 Beckman rotor at 10° C., 35000 rpm, 1 h. The collected virus band was mixed with CsCl 1.35 mg/ml and centrifuged for 18 h as before. The virus band was dialyzed twice against PBS and once against PBS-10% glycerol, and stored at −80° C. Five different bands were seen after the second gradient and every one was purified separately. Viral DNA was extracted from purified virus by EDTA/SDS/Proteinase K treatment, phenol/chloroform extraction, and ethanol precipitation (same conditions as described above for PCR). In ethidium bromide gels, no DNA was detected in the three upper bands and were considered to be mostly empty capsids. The two lower bands contained viral DNA and therefore were full capsids that, by restriction map analysis, were shown to correspond with AdHβ. By PCR with R7+R8 oligos, the deleted packaging signal was amplified from all bands, indicating that virus contained the desired attenuation. With this purified helper virus we test the packaging of different mini-viral vectors. To determine the titer, which is expressed as a number of plaque forming units (PFU) per milliliter of virus in solution, the virus-containing solution was serially diluted in D-MEM 10% FBS (1:10 dilution until 10⁻¹²) and used to infect 293 cells at 90% confluence (0.5 ml/well in 6 well-plates). After 1 h infection at 37° C., the viral suspension was replaced by fresh medium. The next day, cells were overlaid with medium containing 0.5% agarose, 0.025% yeast extract and 5 mM Hepes pH 7.4. Plaques were counted after 6 to 10 days. The titer obtained after amplification and purification of AdHβ was about 10⁹ PFU/ml (virus purified from 20 plates of 150 mm² and resuspended in a final volume of 1 ml). This titer is about 100× lower than that obtained with a similar viral vector containing the wt packaging signal.

1.2 Construction of Plasmids for Mini-viral Vectors:

It has been shown that the linear adenovirus DNA, when covalently circularized head-to to tail by its terminal ITRs can be grown as a plasmid in bacteria but it will replicate and produce virus when transferred into permissive human cells (92). Functional junctions have been naturally selected by transforming bacteria with circular DNA extracted from infected cells. Small deletions in the joints were observed which presumably conferred stability to the plasmids by destroying the perfect palindrome that would result from the head-to-tail fusion of the ITRs of adenovirus DNA. The basic minivirus structure is therefore a plasmid that contains the left end of AdS (including the 103 nt-ITR and the packaging signal until nt 358) fused to the right end of Ad5 (at least including the 103 nt-ITR). The initial approach used to test the mini-viral vector system included the generation of progressive deletions in plasmid pJM17 that contains a functional ITR fusion. pJM17 is a plasmid that contains the entire genome of Ad5 as a DNA molecule circularized at the ITR sequences and a pBR322 derivative, pBRX, inserted in E1A (providing the bacterial replication origin and ampicilin and tetracycline resistant genes) (93). When transfected in 293 cells, which complement the E1A defect, pJM17 replicates but is not packaged because is too large (40.3 kb) to be packaged into the adenovirus capsid (maximum is 38 kb).

Examples of various mini-viral vectors demonstrated in the current literature as well as that of the present invention are described in related patent applications (see Related Applications section, supra). pJM17 was cut with AscI and religated obtaining pBRX-AscI. This removed from mu 43.5 to 70.2 of Ad5 which completely deletes E2A (DNA binding protein) and L3 (hexon, hexon-associated proteins and 23K protease), and partially deletes L2 (penton base and core proteins) and L4 (hexon-associated protein, hexon-trimer scaffold protein, and 33K protein). This deletion abrogates replication and capsid formation from the circular viral DNA, rendering it completely dependent on a helper virus that provides in trans a sufficient quantity of the required replication proteins. pBRX-AscI contains a unique Spe I site at 75.2 mu (L4) into which a 2.7 kb DNA fragment comprising a green fluorescence-protein (GFP) expression cassette was inserted to give M32 (Minivirus of 32 kB). This GFP-cassette is composed of a CMV enhancer/β-actin promoter (CA promoter), the Aequorea victoria GFP cDNA, and a SV40 polyA signal. The use of GFP in the mini-viral vector constructs was utilized in order to determine the presence of the vector in cells using the fluorescence microscopy. Fluorescent microscopy represents one of several methods including but not limited to flow cytometry that may be utilized to detect cells expressing GFP. The presence of AdHβ can be detected by the blue color of X-gal staining. To generate M31, M32 was cut with MluI and religated, this removes from 31.4 to 34.5 mu which partially deletes L1 (52K, 55K and penton-associated proteins). To generate M28, M32 was cut with MluI and AscI and religated, this removes from 31.4 to 43.5 mu which completely deletes L1 and the L2 portion that still remained in M32. To generate M26, M28 was cut with Rsr II and Spe I and religated, this removes from 30.9 to 75.2 mu extending the L1 and L4 deletions. To generate M23, M32 was digested with Nsi I and religated. The Nsi I fragment from 32.2 mu to the CA promoter (with a NsiI site next to the fusion with 75.2 mu), containing the GFP cassette, was religated so the Nsi I site of the CA promoter ligated to 5.5 mu and the Nsi I site at 32.2 ligated at 75.3 mu. This abrogates expression of all proteins between 5.5 to 75.3 mu including E2b (terminal protein, DNA polymerase) and IVa2 proteins. To generate M20, M23 was cut with Mlu I and Asc I, which removes the region from 34.5 to 43.5 mu of the Nsi I fragment of M23, and religated,.

Instead of trimming down a circularized full-length viral genome such as that in pJM17, other mini-viral vectors were constructed by subcloning the minimal cis elements necessary for replication and packaging, including the ITR sequences and the packaging signal, into a small plasmid such as pBluescript (Stratagene) and progressively adding the transgene cassettes and other elements that could improve the therapeutic potential of the viral vector such as elements for episomal maintenance or chromosomal integration. The head-to-tail fused ITRs and the packaging signal next to the left ITR (ITR/ITR+pac)were cut from pBRX-AscI with Eco47III (98.7 mu) and PvuII (1.26 mu) blunted and subcloned into SmaI-EcoR V of pBluescript, respectively. The resulting plasmid, pBS/MiniITR or GT4007, is a 3.8 minivirus plasmid with no expression cassette and several unique restriction sites flanking the ITR/ITR+pac. Using Xho I and Kpn I, the GFP-expression cassette described above was subcloned into pBS/MiniITR to generate M6.5. An internal ribosome entry site (IRES) and a neomycin (neo) cDNA were then subcloned between the CA promoter and the GFP gene to produce M7.9. A similar minivirus was generated comprising neo and GFP in two separate cassettes, M8.5: the Nru I-BstE II fragment from pREP9 (Invitrogen) containing the Tk promoter, neo cDNA and Tk pA, was blunted and subcloned into Stu I-EcoR I of M6.5. M8.5 was used to construct a larger miniAd plasmid in order to test the packaging of miniAd vector with a complete substitution of the adenoviral genome by exogenous DNA. As inserts we used genomic fragments corresponding to the 3′ half of the albumin gene and the 5′ half of the alpha-fetoprotein gene. These fragments were chosen as potential arms with the prospect of studying homologous recombination in this site. We inserted these fragments upstream and downstream of the double GFP/neo expression cassette of M8.5. Therefore, in the resulting construct, pGnE5E3 (23.8 Kb), the GFP and neo transgenes substitute the corresponding 10 Kb albumin-fetoprotein intergenic region present in the human genome. Miniviruses obtained with the plasmids described above are shown in related patent applications (see Related Applications section, supra).

1.3 Generation and Amplification of Minimal AdGFP Vectors:

ADHβ was utilized to support the replication and packaging of the various minimal Ad plasmids. It was important to determine whether the minivirus could be packaged. It was also important to determine whether the size of the minivirus affected the packaging efficiency.

In adenovirus, 100% of the wild type length of DNA is most efficiently packaged, and as the genomic size increases to a maximum of 105% or decreases below 100%, packaging becomes less efficient. A lower limit of 69% (25 kb) has been suggested (94) when wild type adenovirus was used to complement the defective minivirus, but the use of an attenuated helper virus allowed the amplification of a shorter minivirus.

To complement the mini-viruses of the present invention, two methods were utilized that each function with a similar efficacy. In the first method, a CsCl-purified minivirus plasmid was cotransfected with the linear viral DNA extracted from purified AdHβ. Note that the method utilized to purify the viral DNA is subjected to SDS and Proteinase K which destroys the terminal protein responsible for priming replication. This method was utilized to avoid giving the helper virus a replicative advantage over the minivirus plasmid which also lacks the terminal protein. Accordingly, complementation by direct infection with AdHβ did not rescue minivirus. Cotransfection was accomplished using CA₂PO₄ and 2 mg of mini-viral plasmid and 1 mg of viral DNA per well in a 6 well-plate with 293 cells at 50% confluence. After an overnight incubation in the transfection mixture, the medium was changed and the efficiency of transfection was assessed by examination of cells using fluorescence microscopy. With CsCl-purified plasmids this efficiency reached 100% irrespective of the size of the plasmids. Six days post-cotransfection, CPE was observed and virus was harvested from the cells by three cycles of freeze and thaw. In the second method of complementation the minivirus plasmid was cotransfected with pBHG10, a circularized adenovirus plasmid similar to pJM17 incapable of being packaged due to a complete deletion of the packaging signal (95). This plasmid produces all the early proteins necessary for replication as well as the late proteins that form the capsid. When the minivirus is present in the same cell as pBHG10, it will also replicate and, as the minivirus contains the wild-type packaging signal, the miniviral vector will be the major nucleic acid encapsidated. However, when the minivirus is released to the neighbor cells it will not be amplified because is defective. Therefore, to amplify the minivirus, three days after the cotransfection, the cell monolayer was infected with AdHβ at a multiplicity of infection (moi) of 10 plaque forming units (pfu)/cell. Three days after co-transfection, CPE was observed and virus was harvested by three cycles of freeze and thaw.

Regardless of the method of complementation, the lysate (passage 0 of the produced minivirus) was used to infect a fresh monolayer of 90% confluent 293 cells (using 1 to 3 amplification scale). The day after infection, the presence of minivirus was observed by fluorescence and the presence of helper confirmed by X-gal staining. If any helper virus was present in the lysate, further incubation of the cells would lead to the amplification of the mini-virus+helper mixture with the appearance of CPE (the new lysate of this monolayer will be considered as passage 1 of the minivirus). If no helper was present in the lysate, the minivirus alone would not be packaged and only by the addition of new helper would the CPE appear. Therefore, the presence of the helper was assessed by X-gal staining and, with much higher sensitivity, by the appearance of CPE.

Following separate transfections with each of the minivirus constructs (M32, M31, M28, M26, M23, and M20), the appearance of plaques with GFP was observed by fluorescence microscopy. This indicated that the complementation was possible for each of the plasmids tested. Following infection of fresh 293 cells monolayers with the crude extracts of each virus, CPE was observed after 2 days indicating the presence of helper virus. The results of further passage of the minivirus demonstrated that in every passage a 5-fold amplification was produced, and that the packaging efficiency was proportional to the minivirus size. A drop in efficiency of 2 fold per every 3 kb decreased vector size was observed. For example, the efficiency of packaging of M20 would be 2.48% of the wild type (being (36-x kb)/3=n the efficiency is 0.5ⁿ). However, with M6.5, M7.9 and M8.5 no fluorescent plaques were found, indicating very inefficient or absent packaging. This could reflect a packaging lower limit somewhere between 8.5 Kb and 20 Kb. However, it seems more probable that packaging still might take place between these limits but, according to the linear decrease observed, the 11.5 Kb size difference would result in a 7.6 fold less packaging efficiency and amplification may not then be possible.

Complete substitution of the viral genome by exogenous DNA was possible and whether this would affect the packaging efficiency was tested. A completely substituted miniAd of 23.8 Kb containing only the ITRs and the packaging signal of Ad5 was constructed. As exogenous DNA two expression cassettes in tandem, one for GFP and another one for neomycin, were flanked by two long arms of albumin an a-fetoprotein genomic DNA. Packaging was demonstrated by the increasing number of GFP-positive 293 cells when the virus obtained after an initial complementation with AdHb v DNA was passed. This transducing units titer was similar to that obtained with M23, indicating that exogenous DNA did not have a detrimental effect in the packaging efficiency when compared to Ad5 DNA.

1.4 Purification of Minimal AdGFP Virus (M32):

Following passage of the minivirus, the titer increased until all cells became fluorescent following infection. This occurred, for example, at passage 4 of M32. When passage 8 was reached by continuously passing M32 at 1 to 3 amplification scale, enough virus was obtained to infect 75 plates of 150 mm². When CPE was apparent, the virus was extracted by three freeze/thaw cycles and purified by a CsCl gradient as described above. In the gradient four bands were observed, three upper (and therefore lighter) bands and one thicker band in the middle of the centrifuge tube. Every band was collected separately by aspiration from the top of the tube, and dialyzed. Infection of 293 cells with every band and fluorescence observation or X-gal staining demonstrated that the mini-virus and helper virus were both present on the higher density band. Based on the number of green and blue cells, the amount of minivirus and helper was determined to be within the same range. The different size of the viral DNA present in M32 (32 Kb) and in AdHβ (37.1 Kb) should make M32 slightly less dense than AdHβ. To increase the minimal Ad vector to helper ratio the higher density band was separated in a 1.35 g/ml continuous CsCl gradient and fractions were collected from the bottom of the tube. 0.5 ml of every fraction was used directly to infect 293 cells at sub-confluency to check for fluorescent and blue cells after 24 h.

An aliquot of 0.5 μl of every fraction was used to infect one well of a 96 well/plate with 293 cells at 60% confluency. Initial fractions (1 to 6) did not contain M32 or AdHβ (these fractions represent up to 3 ml of the gradient). 100 μl samples of fractions 7 to 16 reveal a large amount of M32 and AdHβ (see panel B for β-gal staining of the same fractions shown under fluorescence in panel A). Subsequent fractions (17 to 29) show a level of M32 similar to the previous fractions but the level of AdHβ is approximately 10 times lower. Therefore, fractions 17-29 represent a 10-fold enrichment of M32 with respect to AdHβ. Following a peak containing large amounts of AdHβ (FIG. 16, fractions 7 to 16), a lighter fraction followed that revealed a 10-fold enrichment for M32 with respect to AdHβ (fractions 17 to 29). Fractionation through CsCl may therefore be utilized to decrease the amount of helper virus present in the minimal Ad preparations.

In summary, the results indicate that the helper used with the partial deletion in the packaging signal taken from the dl18/28 virus is able to complement the large deletions in the mini-viral vector system but it is still packaged in the presence of minivirus. This helper can be used when a pure population of minivirus is not critical, for example in an antitumoral vector system where a minivirus containing several therapeutic genes (for example, interleukins and tumor-suppresser genes) can be combined with this helper containing another therapeutic gene. When higher minimal Ad to helper ratio is required, this helper needs to be further attenuated in its packaging.

### Example 2

### Design of Packaging-Signal Interfered Helper Ad

Since the packaging of adenovirus requires packaging proteins to bind the packaging elements (A repeats) (90, 96), and this invention introduces several specific DNA binding sequences adjacent to Ad5 packaging signals (A repeats) to further physically interfere helper virus packaging function. Two DNA binding sequences have been chosen: A. GAL 4 binding sequence (97); B. tetracycline operator sequence (tetO) (98, 99). GAL4 is a sequence-specific DNA-binding protein that activates transcription in the yeast Saccharomyces cerevisiae. The first 147 amino acids of GAL4 binds to four sites in the galactose upstream activating region UAS_{G} or a near consensus of the naturally occurring sites, the “17-mer” 5′-CGGAGTACTGTCCTCCG-3′ or 5′-CGGAGGACTGTCCTCCG-3′ (97). tetO comes from the Tn10-specified tetracycline-resistance operon of E. coli, in which transcription of resistance-mediating genes is negatively regulated by the tetracycline repressor (tetR) which binds a 19-bp inverted repeat sequence 5′-TCCCTATCAGTGATAGAGA-3′ in tet O (98, 99).

Based on the packaging signal mutation construct GT5000 (Example 1, section 1), a synthetic sequence has been utilized to replace the sequence between Xho I and Xba I (nt 194, 0.5 mu to nt 452, 1.25 mu) of GT5000. Four synthetic sequences have been designed. All four synthetic sequence contain the Ad5 packaging element (A repeats) I, II, VI and VII. Three or four repeats of 17-mer GAL4 binding sequences (5′-CGGAGTACTGTCCTCCG-3′) (97) or 19-mer tetO sequences (5′-TCCCTATCAGTGATAGAGA-3′) (100, 102) were introduced around or between these A repeats. Since the region between A repeats can affect packaging efficiency (90, 96), the distance between each A repeat is maintained as nearly integral turns of the helix, i.e. 10, 21 or 31 bp. The synthetic oligo sequences and positions are described and shown in related patent applications (see Related Applications section, supra).

### Example 3

### Construction and Characterization of Ad-E1 Helper Cell Lines

The majority of adenoviral vectors used in gene therapy applications were designed to have deletions in the E1 region of the adenovirus 5 (Ad5) genome. The E1 region, not including region IX, consists of 9% of the left end of Ad5 (1.2-9.8 map units), and encodes two early region proteins, E1A and E1B. Expression of E1A/E1B is required for virus replication and for expression of all other Ad5 proteins such as E2-E4 and late proteins (100). Deletion of E1 creates a replication-incompetent virus that, in theory, is silent for expression of all Ad5 proteins and expresses only the transgene of interest. Deletion of E1A and E1B is also of interest for safety reasons, since these two proteins, in combination, have been implicated in oncogenic transformation of mammalian cells (101-103). All of the Class I adenovirus vectors used to date in human clinical trials, as well as, the novel packaging-deficient helper virus described in Example 1 are deleted for E1.

E1-deficient adenoviral vectors are propagated in an Ad5 helper cell line called 293 (104). 293 cells were derived by transforming human embryonic kidney cells with sheared fragments of Ad5 DNA. Genomic analysis revealed that 293 cells contain four to five copies per cell of the left 12% of the viral genome (including the entire E1 region) and approximately one copy per cell of 9% of the right end, the E4 region (105). While 293 cells are very efficient at producing high titers of E1-deficient adenovirus, they have the disadvantage that, due to the presence of extraneous Ad5 sequences integrated into the 293 genome (other than the E1 region), recombination can occur with sequences in the E1-deficient adenovirus vector causing the production of E1-containing, replication-competent adenovirus (RCA). Depending on how early a passage the aberrant recombination event occurs during the amplification and propagation of the E1-deficient adenovirus, and which passage is used for large-scale production of the adenovirus stock, production of RCA in 293 cells can present severe ramifications for the safety of human gene therapy trials (106). In addition to production of RCA, recombination in 293 cells can also cause deletions and rearrangements that effect transgene expression, thereby decreasing the titer of functional adenovirus particles. Recently, cell lines have been developed using defined Ad5 DNA fragments, including the E1 region, however these cell lines retain significant sequence overlap with homologous sequences in the E1-deleted adenovirus vectors, which allows for undesirable homologous recombination events and the possibility for generation of RCA (107, 108).

3.1 Construction of Plasmids for Cell Line Generation:

To minimize the possibility of recombination with the adenoviral vector, a novel Ad5 helper cell line has been developed which harbors only the E1A/E1B sequences required for complementation, and does not contain any homologous sequences that overlap with regions in the E1-deficient adenovirus. A 3.1 kb DNA fragment between Afl III (462 bp) and Afl II (3537 bp) sites, which contains sequences encoding for Ad5 E1A and E1B, was cloned in two pieces, sequentially, into the superlinker vector, pSL301 (Invitrogen), as follows: First, an 881 bp Afl III to XbaI fragment (Ad5 bp 462-1343) was cloned from pBRXad5KpnIC1 (a subclone of pJM17) into pSL301 (Afl III/XbaI). Second, a contiguous 2194 bp XbaI to Afl II (Ad5 bp 1343-3537) was cloned from pBRXad5XhoIC1 into the same vector. The resultant 3075 bp E1 fragment (in pSL301) contains the TATA box and RNA cap site for E1A, E1A coding sequence, complete E1B promoter, and E1B coding sequence, including the stop codon for E1B p55 protein, but not including region IX. The 3075 bp Afl III-Afl II E1A/E1B fragment (Ad5 bp 462-3537) was isolated, blunted with Klenow enzyme, and blunt-end ligated into the EcoRV site of the mammalian expression vector, pCDNA3 (Invitrogen), under control of the CMV promoter/enhancer. This process generated an Ad5E1 expression vector, CMV-E1.

3.2 Generation and Characterization of the New Cell Lines:

The CMV-E1 expression vector (including the G418 resistance gene, neo) was transfected using Lipofectamine (Gibco/BRL) into A549 human lung carcinoma cells and G418^{R} colonies were isolated. Single-cell clones were screened for functional E1A/E1B expression. An E1-deleted adenovirus containing a green florescence protein (GFP) expression cassette under CMV/β-actin (CA) promoter, Ad5CA-GFP, was used to infect the A549-E1 clones. Three days post-infection, clones were screened for production of E1-complemented Ad5CA-GFP adenovirus by visual examination for cytopathic effect (CPE). One clone, A549E1-68, displayed 100% CPE in 3 days (similar to that observed for 293 cells). This clone also showed high infectivity, in that virtually 100% of the cells fluoresced green, as determined microscopically, 24 hrs. post-infection. Infection with the E1-deleted adenovirus, Ad5CA-GFP generated a clear area in the center of its plaque, which is evidence of the CPE caused by E1-complemented virus amplification.

The high infection rate as well as rapid generation of CPE induced in this cell line is strong evidence that functional E1A/E1B proteins are being produced which are capable of promoting the replication and amplification of the E1-deleted Ad5CA-GFP virus. Southern Blot analysis using an E1 sequence-specific probe demonstrated the presence of the CMV-E1 transgene in A549E1-68, a subclone of A549E1-68 (E1-68.3), and 293 cells, but not in the parental A549 cell line. The morphology of the E1-transfected cells was significantly different from the parental A549 cell line. A549 cells, at sub-confluent density, grow as distinct single cells with an elongated, fibroblast-like morphology, whereas, the E1 cell line, A549E1-68, grows as colonies of cells with a more cuboidal morphology.

A549E1-68 was compared with 293 cells for production of E1-deleted adenovirus (Ad5CA-GFP) by plaque assay and found to produce an equivalent titer of complemented virus (7×10⁹ PFU for A549E1-68 vs. 9×10⁹ PFU for 293). Immunoprecipitation and Western blot analysis using an E1A specific antibody (M73, Oncogene Science), revealed two E1A-specific bands with apparent molecular weights of 46 kd and 42 kd, corresponding to products expected from E1A 13S and 12S mRNAs (6), and identical in size to those observed in 293 cells. A549E1-68 produced a band of approximately 55 kd using a monoclonal Ab specific for E1B p55. This 55 kd, E1B-specific band, as well as secondary background bands, were observed in 293 cells also. Extra “background” bands found in both experimental and control lanes have been observed by other authors and have been attributed to co-immunoprecipitation of a variety of proteins including, cyclins, p53, and Rb. Unlike A549E1-68 and 293 cells, the parental A549 cell line showed no expression of 46 kd, 42 kd, or 55 kd E1A/E1B proteins. It is clear that A549E1-68 not only expresses E1A and E1B, but that they are functional, since this cell line can complement for production of high titer, E1-deleted, recombinant adenovirus. To prove that this new Ad5 helper cell line can complement without production of RCA, one may serially pass E1-deleted adenovirus on A549E1-68 cells and test the virus amplified during passage, on parental A549 cells for production of E1-containing, replication-competent adenovirus (RCA) by CPE, as well as use PCR primers specific for E1A/E1B sequences. This cell line may be used during propagation and scale-up of all E1-deleted adenovirus vectors, to ensure that production lots are free of RCA.

### Example 4

### Expression Cassette Comprising an FVIII cDNA

The large capacity of minimal Ad vectors of the present invention for the gene of interest allows for insertion of large promoter and protein coding regions that far exceed the size capacity of the conventional Ad vector. It is preferred, for the purposes of the present invention, that the FVIII minimal Ad vector deliver the FVIII gene to the liver. It is, therefore, important to utilize a highly active promoter that functions in the liver. One such promoter is the human albumin gene promoter (32). A 12.5 kb region of the human albumin promoter was obtained from the Dr. Tamaoki from the University of Calgary. Three regions within the 12.5 kb promoter segment have been determined to significantly influence promoter activity (32): 1.) the proximal region comprising the TATA box (550 bp); 2.) an enhancer region at −1.7 kb; and, 3.) a second enhancer region at −6.0 kb. Combined, these regions approximate the strength of the entire 12.5 kb human albumin promoter. The 10.5 kb EcoRI/AvaI fragment of pAlb12.5CAT was co-ligated with the AvaI/HindIII proximal human albumin promoter fragment into the EcoRI/HindIII site of the pBluescript-KS⁺ vector, to generate the recombinant plasmid GT4031. The 7.2 kb full-length human FVIII cDNA with a 5′ flanking SV40 immediate early intron and a 3′ flanking SV-40 poly-adenylation signal was excised from plasmid GT2051 by XhoI/SalI digestion and was cloned into the SalI site of GT4031 to generate plasmid GT2053. The XhoI fragment derived from plasmid GT2033 containing the minimal ITR region and Ad packaging signal was then cloned into the SalI site of GT2053 in either the forward or reverse orientation to generate the albumin/hFVIII minivirus plasmids GT2059 and GT2061, respectively. The restriction enzyme digest patterns of the GT2059 and GT2061 minivirus plasmids are shown in related patent applications (see Related Applications, supra).

The FVIII cDNA may be operably linked to a promoter or transcriptional control element that may be synthetic, controllable or regulatable, or tissue/cell type specific. Preferably, expression of the FVIII cDNA in the producer or helper cell is suppressed during viral production and activated following delivery to a target cell. In this manner, differential expression of the reporter or effector gene of the minimal Ad vector is achieved. Such differentiated expression is accomplished by constructing a DNA molecule having the FVIII cDNA under the transcriptional control of a synthetic promoter such as one having a liver-specific enhancer operably linked to an α₁-antitrypsin (α₁-AT) promoter or one in which the tetracycline operon (tetO) is operably linked to the cytomegalovirus (CMV) promoter (tetO-CMV), in which case a cell line is utilized that expresses the tet-KRAB transcriptional repressor protein.

### Example 5

### Homologous Recombination Arms of the FVIII Expression Cassette

Homologous recombination may be employed to insert an exogenous gene into a the genome of a target cell resulting in stable gene expression. Using this technique, the human FVIII cDNA may be targeted to the genomic DNA of a target cell. Large segments of cellular DNA derived from the human albumin gene or human α-fetoprotein were utilized (32, 33). The 12.5 kb albumin promoter in the FVIII minimal Ad vector functions as the upstream homologous recombination arm while a number of downstream fragments of greater than 6 kb were prepared as potential 3′ recombination arms. The albumin gene, an intergenic region and the α-fetoprotein gene regions useful in the present invention are shown in related applications (see Related Applications, supra). The structure of the expression cassette in plasmid GT2061 comprising the 12.5 kb albumin promoter at the 5′ end and several regions serving as 3′ homologous recombination arms are shown in related applications (see Related Applications, supra). These vectors serve as homologous recombination replacement vectors since the orientation of the arms are in identical orientation as the sequences in the normal human genome. A construct (GT2063) comprising the 3′ XhoI recombination arm derived from the human albumin gene and the pAlb-E5 segment cloned into the unique SalI site of GT2061 is shown in related applications (see Related Applications, supra). Restriction enzyme digestion of the appropriate vectors for construction of the human albumin promoter-driven hFVIII adjacent to a 3′ albumin homologous recombination arm is shown in related applications (see Related Applications, supra). Plasmid GT2063 was constructed by insertion of the XhoI albumin gene fragment of plasmid pE5 into the unique SalI site of GT2061.

### Example 6

### Generation of FVIII Minimal Ad Vectors

The 12.5 kb EcoRI/HindIII human albumin promoter fragment was inserted into pBluescriptKS⁺ (Stratagene, La Jolla, Calif.). The human albumin promoter vector, GT4031, thus contains a unique SalI site into which the human FVIII cDNA (the region in GT2051 from XhoI to SalI comprising the SV40 early intron at the 5′ end and the SV40 polyadenylation signal at the 3′ end) was inserted. The resulting plasmid, GT2053, contains unique SalI and XhoI sites located 3′ to the polyadenylation site. The Ad minimal ITR and wild type packaging sequence was excised from plasmid GT2033 by XhoI digestion and cloned into the SalI site of plasmid GT2053 to generate plasmid GT2061. The 6.8 kb arm of the albumin gene was isolated from pAlb-E5 and cloned into the unique SalI site of GT2061 to generate plasmid GT2063. GT2063 was transfected into 293 cells together with the helper virus DNA to generate the minimal Ad FVIII minivirus designated GTV2063.

To generate the FVIII minivirus, the helper-virus genome (2 μg) was purified from virus particles and co-transfected with the helper Ad genome (0.2 μg) into 293 cells by calcium phosphate-mediated transfection (81). Following the appearance of CPE, cell-free freeze thaw lysates were prepared and utilized to infect fresh 293 cells. Human FVIII, indicating the presence of the GT2063 minimal Ad, was detected in the cell supernatants using the Coatest FVIII chromogenic assay (Pharmacia). The data are consistent with propagation of a helper/GT2063 minimal Ad vector mixture.

In yet another approach, the adenoviral helper plasmid, pBHG10, which lacks the Ad packaging signal and E1 region but encodes the remainder of the Ad proteins, was co-transfected with the minimal Ad clone GT2063 into 293 cells. Rescue of the Ad-minivirus genome was achieved following infection of 293 cells with an E1-substituted helper virus having attenuated packaging function. Both the Ad-helper and minimal Ad genomes may be packaged, and adenoviral particles carrying either genome may be generated using the methodologies of the present invention, although the helper Ad/minimal Ad ratios is variable.

Generation and detection of the FVIII minimal Ad is described in related patent applications (see Related Applications section, supra). Briefly, helper plasmid pBHG10 (0.2 μg) and the minimal Ad vector comprising the human FVIII gene (GT2063; 2 μg) were co-transfected into 293 cells by calcium phosphate transfection (81). Transfection into 293 cells may be performed using any of the well-known and widely available techniques such as lipofection (i.e., using Lipofectamine from GIBCO/BRL) or electroporation (i.e., using reagents and electroporator available from Bio-Rad). Infection of the transfected 293 cells with an attenuated helper virus was performed three days after transfection. A cytopathic effect (CPE), indicating adenoviral infection has progressed sufficiently, was observed four days after infection. Viral stocks (passage 0 or P0) were then prepared by multiple freeze-thaw of the infected cell pellets. 293 cells were then infected with P0 stocks (1:1) and supernatants collected 24 hours post-infection were positive by PCR specific for the presence of hFVIII sequence. Six days later, a CPE was detected and freeze-thaw lysates (P2) prepared. The P2 lysate was then tested by PCR for the presence of packaged GT2063 minimal Ad and for functional hFVIII. Expression of hFVIII in 293 cells was expected to be minimal because the human albumin promoter is not very active in these cells. This has been determined using both CAT assays (69) and an FVIII chromogenic assay (Helena Laboratories, Pharmacia) following transfection of 293 cells with GT2061 using the calcium phosphate precipitation transfection method.

### Example 7

### Amplification and Purification of the Minimal AdFVIII

The applicants have previously filed U.S. patent application Ser. No. 08/658,961 on May 31, 1996 and provided within that application the reagents and methodologies for generating the minimal AdFVIII vector GT2063 and performing the initial passages of propagation (79, 80). In these propagation rounds, the applicants found that the ratio of minimal AdFVIII vector to helper virus, AdHβ, increased as propogation progressed. Previous patent applications further provide analyses of the vector to helper ratios performed using PCR and Southern blot, both conventional techniques well known to one skilled in the art (see Related Applications section, supra). Briefly, for every passage, 500 μl of crude extract of virus (obtained by three freeze/thaw cycles of infected cells) was used to infect a new subconfluent monolayer of 293 cells in a well of a six-well plate. 1 hour after infection 1.5 ml of fresh medium (DMEM/10% FBS) was added. Upon completion of cytophatic effect (CPE), the cells were harvested and the virus extracted again. In each passage, 0.5 ml of the 2 ml were used so the amplification scale of this propagation was 1 to 4. The cleared crude lysate of every passage was used to purify viral DNA by SDS/EDTA/Proteinase K digestion and ethanol precipitation. The viral DNA was used for PCR and Southern blot to detect minimal Ad and, independently, helper Ad virus DNA.

PCR was performed using primers specific to human FVIII cDNA and amplifications were performed on virus subjected to DNAse treatment prior to DNA extraction to remove any residual non-viral contaminating plasmid DNA. PCR was performed using isolated viral DNA as template (1/20 of the viral DNA isolated), FVIII primer #1 at a final concentration of 1 μM (SEQ ID NO:1; ACCAGTCAAAGGGAGAAAGAAGA), FVIII primer #2 at a final concentration of 1 μM (SEQ ID NO:2; CGATGGTTCCTCACAAGAAATGT), and the following conditions: annealing for one minute at 55° C., polymerization for one minute at 72° C., denaturation for one minute at 94° C. for a total of 35 cycles. The results indicated that the FVIII minivirus was present in early passages (passage 3).

The results of PCR amplification of the packaging signal of the FVIII minimal Ad and, independently, the helper Ad are described in related patent applications (see Related Applications section, supra). PCR was also performed on the packaging signal region. Briefly, PCR was performed using isolated viral DNA (1/20 of the total viral DNA isolated) as template, packaging signal primer #1 (SEQ ID NO:3; GGAACACATGTAAGCGACGG) at a final concentration of 1 μM, packaging signal primer #2 (SEQ ID NO:4; CCATCGATAATAATAAAACGCCAACTTTGACCCG) at a final concentration of 1 μM and the following conditions: annealing for one minute at 55° C., polymerization for one minute at 72° C., denaturation for one minute at 94° C. for a total of 35 cycles. As the packaging signal of the helper Ad is partially deleted, the PCR product from the packaging signal deleted helper is shorter (177 bp) than that of the minimal Ad having a wild-type packaging signal (approximately 310 bp). In the initial passages, the FVIII miniAd was not detected but its presence was increasingly detected in passages 3 to 6. Identical results were obtained using Southern blot analysis. As a probe in the Southern blot analysis, an Ad DNA fragment adjacent to the right ITR present in both the FVIII minimal Ad and the helper Ad was used. The expected length of the detected fragments after Pst I digestion of the minimal Ad GT2063 and the AdHB is 3.3 and 2.2 Kb, respectively. A compilation of four Southern blots (A-D) of FVIII minimal Ad DNA independently isolated from passages 1 to 21 is shown in related applications (see Related Applications section, supra). The 3.3 Kb band corresponding to minimal AdFVIII was detected in DNA isolated from passage 5-21. A steady increase in FVIII minimal Ad DNA was detected until passage 10 which was followed by progressive decrease in FVIII minimal Ad until passage 12. This cycle of increasing and decreasing levels of FVIII minimal Ad DNA was observed to occur approximately every four passages and was accompanied by a parallel cycle of the level of helper Ad DNA, which has a slightly earlier onset. To better define these cycles, the amount of FVIII minimal Ad DNA and helper Ad DNA was quantified densitometrically from the Southern blots and plotted. The intensity of the bands from equal amounts of marker (1 Kb ladder marker from Gibco, Gaithersburg, Md.) were used to normalize the results of the different blots. The observed cycles match with the well known dynamics of a virus population generated in association with a defective interfering virus (in the system of the present invention, the virus population comprises the FVIII minimal Ad) and a helper virus. The understanding and control of these cycles is important to determine at which passage the minimal Ad vectors should be purified to obtain optimal titers. Passages such as #18 (P18) result in a vector preparation enriched for the FVIII minimal Ad (i.e., P18 appears to contain 10 times more FVIII minimal Ad than helper Ad), albeit at a low titer. Passages such as #20 (p20), comprise high levels of FVIII minimal Ad and helper Ad, although at an undesirable FVIII minimal Ad to helper Ad ratio of 1:1.

A large scale amplification was performed at p20. One hundred 15-cm dishes, each comprising approximately approximate 10⁹ infected 293 cells (ATCC# CRL1573) were harvested upon completion of the CPE. A crude lysate was then prepared by three freeze/thaw cycles to extract the virus. The crude lysate was cleared by centrifugation, loaded onto a step density gradient of CsCl (three layers of 1.5, 1.35, and 1.25 g/ml) and centrifuged at 35000×g for 1 h. The band corresponding to the mixture of minimal Ad and helper Ad was further purified using a second continuous CsCl gradient of 1.35 g/ml. After 16 h centrifugation at 35,000 rpm (150,000×g), two bands of similar intensity were observed, isolated separately, and dialyzed into phosphate buffered saline (PBS) containing 10% glycerol. Viral DNA was purified from an aliquot of each band and the amounts of minimal AdFVIII and Helper analyzed by Southern blot. The upper band (lighter) appeared to comprise mainly minimal Ad and the lower band mainly helper Ad. These results were expected as the FVIII minimal Ad genome (GT2063-31 Kb) is smaller than the helper Ad genome (AdHβ=37.1 Kb), consistent with previous CsCl fractionation results for the M32 minimal Ad demonstrated in a related application (U.S. Ser. No. 08/658,961, filed May 31, 1996) of the present application.

### Example 8

### Test of the Minimal AdFVIII in Cell Lines

The FVIII minimal Ad (GT2063) was purified by CsCl as described above and utilized to demonstrate production of FVIII in host cells infected with the vector. To this end, 293 and HepG2 cells were utilized due to their known ability to utilize the albumin promoter. FVIII production in these cells was assayed by immunohistochemistry and functional assays 24 h after infection. Purified FVIII minimal Ad vector was added to 0.5 ml of medium and used to infect 6×10⁵ 293 and HepG2 cells in a 4 cm² well. After a 4 h incubation to allow for adsorption of the viral particles to the host cells, the infection medium was replaced with fresh medium. Following infection of 293 cells with 0.1 μl of a 1/100 dilution of the lighter fraction comprising the FVIII minimal Ad and immunohistochemical analysis of the cells for the presence of human FVIII, approximately 10% of the cells stained positive for FVIII expression.

293 cells were grown in chamber slides and infected with a diluted (1/100) 1 μl aliquot of the upper or lower fractions. Twenty-four hours following infection, the cells were fixed and stained with a FVIII specific mAb (Cedar Lane Sheep anti-human FVIIIC, #CL20035A, Accurate Chemical and Scientific Corporation, Westbury, N.Y.) and subsequently a secondary antibody (biotinylated donkey anti-sheep IgG, Jackson Immunoresearch, #713-065-147) and DAB (resulting in a reddish-brown color; SIGMA Cat. No. D7679). Transduction with two independent preparations of the upper fraction (minimal Ad FVIII enriched). Ten percent of the cells stained positively for FVIII expression. Transduction with the lower fraction (helper virus enriched) resulted in 0.1% of the cells staining positive for FVIII expression.

The estimated titer in transducing units per milliliter was determined to be 6×10⁹ transducing units/ml. If an adsorption time of 4 h, an adsorption volume of 0.5 ml in 4 cm², and a non-rocking adsorption are taken into account, the estimated titer may be reduced by a factor of 0.42, 0.56, and 0.53, respectively (49). The actual titer of minimal AdFVIII vector would therefore be estimated to be 4.6×10¹⁰ transducing units/ml. The titer determined by optical absorbance at 260 nm, which reflects the number of viral particles per milliliter was determined to be 3.6×10¹² particles/ml for the lighter fraction of FVIII minimal Ad. Therefore, the bioactivity of the FVIII minimal Ad can be calculated to be one FVIII-transducing unit per every 78 viral particles, which falls within the levels of acceptability recommended by the Food and Drug Administration (49).

The amount of functional FVIII in the supernatant of transduced cells was determined using the chromogenic Coatest FVIII Test (Pharmacia, Piscataway, N.J.). A Coatest chromogenic assay for functional FVIII was performed. A standard curve in triplicate from 4000 ng/ml to 62.5 ng/ml was plotted to obtain the equation to extrapolate the readings from the samples. Experiments were performed in triplicate: 10 μl aliquots of miniAdFVIII in 293 cells; 1 μl aliquots of miniAdFVIII in 293 cells; 10 μl aliquots of miniAdFVIII in HepG 2 cells; 1 μl aliquots of miniAdFVIII in HepG 2 cells; conditioned medium from untransduced 293 cells; and conditioned medium from untransduced HepG 2 cells were independently tested. One million 293 cells or, independently, HepG2 cells, were infected with an excess amount of purified vector in order to achieve 100% transduction. Infection conditions were as described above and the supernatants (2 ml) were collected 24 h after infection. To generate a standard curve, a standard human plasma sample was serially diluted in cell culture medium to obtain a final FVIII concentration range from 62.5 to 4000 ng/ml. The results are shown in FIG. 41. The amount of FVIII detected in HepG2 and 293 supernatants were 0.8 and 0.23 ng/ml respectively. Therefore, the total amount of FVIII produced in 24 h was 1.6 ng per million HepG2 cells and 0.46 ng per million 293 cells.

### Example 9

### Improvement of the Minimal AdFVIII in Vivo

Improvements in the vector system were accomplished by generation of a vector into which various expression cassettes may be cloned. The vector GT2063 was modified by excising the proximal albumin promoter region and human FVIII gene localized between the Pme I and SalI sites. This was accomplished by first converting the Pme I site of GT2063 to a Sal I site by ligating a SalI linker to the Pme site. The resulting clone, GT2072, was treated with Sal I and religated to remove the proximal albumin promoter/hFVIII gene region thereby creating a minimal Ad vector having a unique Sal I cloning site for insertion of various expression cassettes. The expression from such cassettes may be affected by albumin gene enhancers located upstream. Each clone was analyzed to determine the level of expression of the transgene.

Expression cassettes were prepared for insertion into the improved vector, GT2072. The expression cassettes of this example comprises the cytomegalovirus (CMV) immediate early promoter, the elongation factor I (EF-I) promoter (which are known to function in a wide variety of cell types) or the liver-specific promoter for the phosphoenol pyruvate carboxykinase (PEPCK) gene. The EF-I and CMV promoters were each separately utilized to drive expression of either the full length FVIII cDNA or the B-domain deleted (BDD) factor VIII cDNA. An EF-I BDD FVIII cassette flanked by Sal I sites was then cloned into the Sal I site of GT2073 resulting in generation of the plasmid. An expression vector comprising the full length human FVIII coding region under control of the CMV promoter was also constructed.

### Example 10

### Construction of an Integratable AAV-ITR/Rep System-based Vector

Adeno-associated virus (AAV) is a human non-pathogenic single-stranded linear parvovirus that replicates only in the presence of a helper virus like adenovirus or herpes virus. However, in the absence of helper, AAV can integrate specifically in the host genome and be maintained as a latent provirus (34). The particular locus where AAV integrates has been located to chromosome 19q13.3-qter and named AAVS1 (22-25, 35).

The mechanism of AAV integration has not been fully elucidated. However, two viral elements have been implicated in this process: the AAV ITRs and two forms of the Rep viral proteins (Rep78 and Rep68). The AAV ITRs (Inverted Terminal Repeats) are palindromic sequences present in both ends of the AAV genome, that fold into hairpin structures and function as origins of replication. Several activities have been described for Rep78/68 proteins including sequence-specific DNA binding (36, 37), sequence and strand-specific endonuclease activity (38), and ATP-dependent helicase activity (38-40). These proteins can bind to a specific sequence in the ITR DNA and promote the process named terminal resolution by which the ITR hairpin is nicked and replicated. A Rep-binding motif and a terminal resolution site (trs) have been identified in both the AAV ITR and AAVS1 and demonstrated to promote in vitro DNA replication in the presence of Rep (28). It has also been shown that Rep68 protein can mediate complex formation between the AAV ITR DNA and AAVS1 site in vitro (41). These findings suggest a model in which the DNA binding and endonuclease activity of Rep along with limited DNA synthesis at the ITRs and AAVS1 sites would allow targeted integration of the sequences contained between the ITRs (27).

AAV has been considered as a candidate vector for gene therapy. However, the limited size of exogenous DNA that it can accept (4.2 Kb), the difficulty in getting high titers in large-scale preparations, and the loss of specific integration of the recombinant AAV have posed problems for the use of this virus as a gene therapy vector.

10.1 Construction of Plasmids to Test AAV/ITR-Rep Integration System

Towards the incorporation of the AAV integration machinery in a minimal Ad vector, the inventors have developed and tested a plasmid vector that contains the adeno-associated viral elements necessary for integration. In a design of the present invention, the vector consists of a Rep expression cassette (containing the viral endogenous promoter), as well as a cassette for expression of a reporter gene flanked by two AAV ITRs. The Rep expression cassette was obtained after PCR amplification of sequences 193 to 2216 in the AAV genome from plasmid pSUB201 (41). This fragment starts right after the ITR and extends through the p5 promoter and the Rep78 coding sequence.

10.2 Test of AAV/ITR-Rep Integration System in Culture Cells:

Expression of Rep from this plasmid in transiently-transfected 293 cells and an E1 non-expressing cell line (Chang liver cells) was tested by immunoprecipitation plus Western blot with specific antibodies. The results showed that two different forms of Rep are produced in 293 and Chang liver cells. Rep78 appeared as a doublet while Rep52, product of p19 promoter, appeared as a single band. In Chang liver cells, two major forms are detected, Rep78 also as a doublet and Rep52, although the signal is stronger in 293 cells.

In order to test for the specific integration capability of these plasmids, a control plasmid was constructed by removing the Rep expression cassette, but keeping the reporter gene expression cassette placed between two AAV ITRs. 293 cells were transfected with plasmids GT9003 or GT9004 and then selected for 12 days with G418 (0.5 mg/ml). G418resistant colonies were isolated, expanded, and genomic DNA was extracted from different colonies by the salt precipitation method (125). Genomic DNA was digested with EcoRI and analyzed by Southern blot with a probe for AAVS1. EcoRI was chosen because the AAVS1 locus is contained within an 8 Kb EcoRI-EcoRI fragment. Results indicated that 50% of the resistant colonies analyzed which derived from plasmid GT9003 (Rep-expressing plasmid) revealed rearrangements of at least one AAVS1 locus, as indicated by the presence of a shifted band in addition to the 8 kb band corresponding to the normal sequence. Rearrangements were not observed in the colonies derived from plasmid GT9004, indicating that this phenomenon is dependent on the expression of Rep. These results suggested that Rep was able to drive specific integration of the transgene. The membrane was then rehybridized to a specific probe for neo. The pattern of bands obtained indicated that some AAVS1 rearrangements correspond to neo (e.g., clone 2L2) but also suggested that random integration events occurred frequently in the clones analyzed, possibly favored by the selective pressure applied.

10.3 Test of AAV/ITR-Rep Integration System Without Selective Pressure:

In order to rule out this possibility, another set of experiments were performed with plasmids GT9012 and GT9013. In these plasmids, the reporter gene is GFP (Aequorea victoria green fluorescent protein). This reporter makes cells suitable for isolation using methods including but not limited to sorting and single-cell cloning by flow cytometry, thereby eliminating effects of selective pressure imparted by the neo expression cassette. 293 cells were transfected with either plasmid. One day after transfection, cells falling into a given range of fluorescence (thus eliminating variability due to differences in transfectability) were sorted by flow cytometry and single-cell cloned in 96-well plates. Two to three weeks after sorting, colonies were scored for fluorescence. Three independent experiments were performed and the results are shown in Table 2-1. The cloning efficiency (number of colonies developed per total number of seeded wells) showed some variability for GT9013-derived cells, but was generally constant for those transfected with plasmid GT9012. Approximately 50% of the colonies derived from plasmid GT9012 were fluorescent and maintained their fluorescence in subsequent passages, whereas 8% of those derived from plasmid GT9013 showed any fluorescence. The fluorescence intensity was dim, an observation consistent with the integration of one or few copies of the GFP expression cassette into the host cell genome. Interestingly, some colonies showed a mosaicism in GFP expression. One explanation for this could be that the integration event occurred after the sorted cell started division giving rise to two different populations. In a parallel experiment cells were transfected with plasmid pCA-GFP that contains a GFP expression cassette alone (no viral sequences). Fluorescent colonies were not detected after sorting plus single-cell cloning (Table 2-1). Taken together, these results indicate that the efficiency of integration is enhanced by the presence of AAV ITRs but is 4-5 fold higher when Rep is expressed. To further analyze targeted integration of GFP in AAVS1, several colonies (fluorescent and non-fluorescent) were grown and genomic DNA was extracted as described above. Results were obtained by Southern blot with a probe for AAVS1. Rearrangements of AAVS1 were detected in several colonies derived from plasmid GT9012 whereas no rearrangement is observed in GT9013-derived colonies, thus indicating that the presence of Rep is necessary for targeted integration. This membrane was then probed for GFP to check the correspondence with the rearranged bands. Parental cell line 293 was negative, as expected. Five clones showed bands over 8 kb matching those obtained with AAVS1, therefore indicating specific integration of GFP in AAVS1. Altogether, the results shown above indicate that plasmids containing AAV ITRs and Rep can integrate at high frequency in the host genomic DNA and suggest that this design is useful for the integration of sequences delivered by adenoviral vectors.

TABLE 2-1
Results of single-cell cloning experiments.

| | | GT9012 | GT9013 | pCAGFP |
|---|---|---|---|---|
| Experiment 1 | Cloning | 92/192 | 116/192 | |
| | efficiency* | (48%) | (60%) | |
| | Integration | 43/92 | 5/116 | |
| | efficiency** | (47%) | (4%) | |
| Experiment 2 | Cloning | 42/96 | 26/96 | |
| | efficiency | (44%) | (27%) | |
| | Integration | 23/42 | 2/26 | |
| | efficiency | (55%) | (8%) | |
| Experiment 3 | Cloning | 93/192 | 25/192 | 51/96 |
| | efficiency | (48%) | (13%) | (53%) |
| | Integration | 37/93 | 1/25 | 0/51 |
| | efficiency | (40%) | (4%) | (0%) |

*indicates number of colonies per number of wells seeded.
**indicates number of colonies showing fluorescent cells two weeks after sorting per number of colonies.

### Example 11

### Site-specific Integration of the FVIII Expression Cassette

It has been previously shown by the applicants in a related application (U.S. patent app. Ser. No. 08/658,961, filed on May 31, 1996) that introduction of ITR DNA sequences in a plasmid coupled with Rep78 expression enhances the integration of DNA sequences of interest into the cellular genome (56). The invention described in U.S. patent app. Ser. No. 08/658,961 comprises multiple plasmids comprising an expression cassette having a reporter gene [i.e., neo or the gene encoding green fluorescent protein (GFP)] flanked by AAV ITR sequence (hereafter referred to as the integration cassette), in combination with an upstream Rep expression cassette. Experimental results demonstrate that the integration frequency of these plasmids (i.e., GT9003 and GT9012) is approximately 10 times higher than those plasmids lacking the Rep cassette (i.e., GT9004 and GT9013). The data further indicates that Rep is essential for efficient, targeted integration of exogenous DNA into a host cell genome. In light of these results, the present invention provides a hybrid vector that combines the advantages of the Ad vector (high titer preparation, large capacity for exogenous DNA, high level infectivity of multiple cell types) and the integration capabilities of AAV. This hybrid virus of the present invention replicates as an adenovirus and comprises the AAV elements sufficient for integration.

The present invention comprises a minimal Ad vector having a Rep expression cassette and a FVIII expression cassette flanked by AAV ITRs. Additional exogenous DNA (up to 36 kb) may be inserted into the vector. Additional exogenous DNA of this vector corresponds to human albumin genomic sequences (non-coding). The Rep expression cassette encompasses bp 193 to 2216 bp of the AAV genome. This fragment originates immediately following the ITR and extends through the p5 promoter and the Rep78 coding sequence. For the reasons listed below, a fragment comprising seven tet operators was introduced upstream of the p5 promoter was included to allow for transcriptional repression of the rep gene by the tet-KRAB repressor (42).

It is possible that high levels of Rep protein within a cell would be cytostatic and possibly interfere with replication of the minimal Ad vector. The tet-KRAB repressor, then, may be provided as a transcriptional switch in order inhibit expression of Rep during viral vector generation. To this end, the present invention provides a 293 cell line stably expressing the tet-KRAB repressor protein. Upon entry of virus into the host cell that does not express the tet-KRAB repressor protein, Rep expression occurs due to the absence of the repressor in those cells, thus promoting integration of the sequences flanked by AAV ITRs into the cellular genome. The viral vector thus generated may be tested in vitro and in vivo for the frequency and specificity of integration.

### Example 12

### AAVS1 Cloning and Vector Construction

An embodiment of the present invention is a methodology for the generation of a transgenic mouse harboring the human AAVS1 integration site for use as an in vivo animal model. The animal model is to be used for testing site-specific integration of a viral vector containing the AAV integration mechanism described above. The first step towards development of the animal model was cloning of the AAVS1 site and insertion of the sequence into a mammalian expression vector.

The AAVS1 human integration site was originally cloned by Kotin et. al. (50) as an 8.2 kb EcoRI fragment, of which the first 4067 bp have been sequenced. This DNA sequence information was used to design two oligonucleotide primers, which were subsequently used to generate a 253 bp PCR product for use as an AAVS1-specific probe. The upper primer, U2492 (SEQ ID NO:5: GCTGTCTGGTGCGTTTCACTGAT), is a 23-mer that extends from basepairs 2492-2515 of the AAVS1 sequence and the lower primer, L2722 (SEQ ID NO:6: TCACAAAGGGAGTTTTCCACACG), also a 23-mer extends from basepairs 2722-2745 of the AAVS1 sequence. PCR amplification was performed using 100 ng human genomic DNA as template and a 1 μM final concentration of the U2492 and L2722 primers (SEQ ID NO:5 and SEQ ID NO;6, respectively) as follows: 95° C., 4 min—1 cycle; 95° C., 0.5 min, 55° C., 0.5 min, 72° C., 1 min—35 cycles; 72° C., 7 min—1 cycle. The 253 bp AAVs1-specific PCR product was sent to Genome Systems (St.Louis, Mo.) where it was used to screen a human P1 genomic DNA library. Four P1 clones (#6576, 6577, 6578, 6579), which ranged in size from 80-120 kb, were identified which yielded the correct 253 bp AAVS1 PCR fragment. To confirm that these clones contained the AAVS 1 sequence, DNA was isolated from the 4 P1 clones, digested with either EcoRI only or EcoRI in combination with EcoRV, and used for Southern blot analysis. Hybridization of the blot using the 253 bp AAVS1-specific PCR product as a probe, revealed that all clones contained the expected 8.2 kb EcoRI fragment and the expected 5.2 kb EcoRI/EcoRV fragment, indicating that they contained an intact, full-length copy of the human AAVS1 sequence. The 8.2 kb EcoRI fragment was isolated from P1 clone #6576 and cloned into the EcoRI site of the mammalian expression vector, pGKneo. The resulting 12.9 kb plasmid, pAAVS1-Neo, harbors the neomycin resistance gene (Neo) for selection in mammalian cells, as well as the human AAVS1 sequence.

### Example 13

### Generation and Characterization of AAVS1 (+) ES Cell Lines

To generate a mouse embryonic stem (ES) cell line comprising the AAVS1 sequence for use in the generation of an AAVS1 transgenic mouse, the pAAVS1-Neo plasmid was transfected into mouse ES cells (129 Sv agouti, Genome Systems). 25 ug of pAAVS1-Neo plasmid DNA was linearized with XbaI and transfected by electroporation (975 uFd, 252 v.) into ES cells using a Biorad Gene Pulser. Transfected cells were selected for one week in 250 ug/ml G418. 24 neo-resistant (Neo^{R}) colonies were isolated, expanded, and characterized by morphology to obtain clones which were >95% “undifferentiated” in order to enrich for cell lines that maintained a totipotent phenotype. Genomic DNA was isolated from 17 Neo^{R} ES clones, as well as from the untransfected, parental ES cell line, and 100 ng of the DNA utilized as template using primers U2492 and L2722 (SEQ ID NO:5 and SEQ ID NO;6, respectively; final concentration of 1 μM) for AAVS1-specific PCR. The conditions for PCR were: 95° C., 4 min—1 cycle; 95° C., 0.5 min, 55° C., 0.5 min, 72° C., 1 min—35 cycles; 72° C., 7 min—1 cycle. PCR of DNA from 17/17 Neo^{R} ES clones generated the expected 253 bp AAVS1 PCR product, while PCR analysis of DNA from the untransfected control ES cells did not generate detectable PCR product. Southern blot analysis was performed on control and AAVS 1 (+) ES cell lines to confirm that a functional AAVS 1 sequence had been preserved following transfection and genomic integration. Genomic DNA from each of the AAVS1 positive (as assessed by PCR) ES cell lines (AAVS1 ES#4 and ES#3.16) was digested with EcoRI in combination with EcoRV, electrophoresed, blotted, and hybridized with an 8.2 kb AAVS1 probe. Both ES#4 and ES#3.16 cell lines contained the expected 5.2 kb and 3.0 kb EcoRI/EcoRV fragments, indicating integration of the entire 8.2 kb AAVS1 sequence. The untransfected parental ES cells showed no hybridizing bands using this human AAVS1-specific probe. It was expected that some hybridization would be detected in the control mouse ES cells, since it is known that the AAV virus integrates into the mouse genome as well as in human, however, the mouse homologue for AAVS1 (which has not yet been identified) must be significantly divergent from the human sequence in that it does not cross-hybridize with a human AAVS1 probe. Each of these AAVS1 (+) cell lines, ES#4 and ES#3.16, were used for subsequent blastocyst microinjection experiments towards the production of AAVS1 transgenic mice.

### Example 14

### Production of AAVS1 Transgenic Mice

The AAVS1-positive ES clones, ES#4 and ES#3.16, were grown on 1° murine embryonic fibroblast feeder layers, in the presence of Leukemic Inhibitory Factor (LIF-an anti-differentiation factor), and maintained at very low passage (P.2-P.7) in order to preserve an undifferentiated, totipotent phenotype. Blastocyst-stage embryos were collected at Day 3.5 p.c. from superovulated, C57BL/6 mice, maintained in M16 embryo medium. 15-20 ES cells (AAVS1 ES#4 or ES#3.16) and microinjected into the blastocoel cavity of the 3.5 day embryos using a Leitz DM-ILB Microinjection Workstation. Following microinjection, the embryos were returned to M16 medium and incubated in 5% CO₂, 37° C. for 2 hours to allow the blastocysts to re-cavitate. 10-15 injected blastocysts were subsequently transferred into the uterus of Day 2.5 post-coitus (p.c.), pseudopregnant, CB6F₁ foster mothers. Following uterus transfer, the blastocysts implant into the uterine wall, the AAVS1-positive ES cells become incorporated into the embryo's inner cell mass, and contribute their genetic information to the developing embryo, resulting in the birth of transgenic (chimeric) progeny approximately 17 days later. To date more than 40 high-percentage, male chimeras (founders) have been produced. Examples of some of these chimeric founders, demonstrating high-percentage contribution of the ES cell-derived agouti (brown) coat-color genes were obtained. PCR analysis was performed for detection of the AAVS1 transgene in chimeric mice. Genomic DNA was isolated from the tails of AAVS1 chimeras and from non-chimeric littermates, and 100 ng of the DNA screened by PCR analysis using the AAVS1-specific primers, U2492 and L2722 (SEQ ID NO:5 and SEQ ID NO;6, respectively) at a final concentration of 1 μM. The conditions for PCR were: 95° C., 4 min—1 cycle; 95° C., 0.5 min, 55° C., 0.5 min, 72° C., 1 min—35 cycles; 72° C., 7 min—1 cycle. The correct 253 bp AAVS1 PCR product was indeed detected in tail DNA from a high-percentage chimera, but was not detected in the tail DNA of a non-chimeric littermate or in a low percentage chimera with less than 10% agouti coat color chimerism. Thus human AAVS1 integration sequence has been successfully cloned, and transfected into mouse embryonic stem (“ES”) cells. The transfected ES cells were then microinjected into blastocyst-stage embryos, and demonstrated the presence of this human transgene in the genome of the resultant transgenic mice. These chimeric founders are then bred with wild-type C57BL/6 females to obtain germline transmission of the human AAVS1 transgene. Once germline transmission is achieved, the F₁ heterozygote progeny are bred resulting in a homozygous AAVS 1 transgenic mouse line. This homozygous line may then be used to test AAVS1 site-specific integration of either AAV viral vectors, hybrid adenovirus/AAV viral vectors, or any other plasmid vector comprising the AAV ITRs and Rep 78/68 genes necessary for integration at the AAVS1 site. The AAV transgene vectors may be delivered in vivo to the AAVS1 transgenic mice either by viral infection (following intravenous injection) or by using ligand-mediated DNA/liposome complexes. The frequency of site-specific integration, stability of the integrated transgene and the duration of stable protein expression (i.e., human Factor VIII, Factor IX, and the like) may then be assessed following integration into the target cells.

The efficiency of in vivo delivery of a viral vector including the adeno-associated integration element to a transgenic mouse having the AAVS1 sequence incorporated into its genome comprising may be tested by the following method. A viral vector of the present invention is injected into the intravenous or portal vein of the transgenic mouse. The vector may be part of a pharmaceutical composition and may or may not be complexed with lipid such as Lipofectamine (GIBCO/BRL) and/or a liver-specific ligand (79, 80). Following administration of the viral vector to the mouse, blood samples are taken weekly for up to a year or more from the tail vein to assess the duration of transgene expression. The level of expression of the effector or reporter gene of the viral vector is measured using a technique such as northern blot, RNase protection analysis, or PCR. In testing the FVIII minimal Ad, FVIII is detected by ELISA assay. The level of expression of the effector or reporter gene in each blood sample is compared to one another in order to determine the duration of transgene expression. Also, in order to determine site-specific integration of the vector, genomic DNA is isolated from the liver tissue of the animal. PCR analysis of the genomic DNA using an AAVS1-specific primer and a primer containing sequence homologuos to sequence of the vector is then performed. Site-specific integration of the vector at the AAVS1 site of the genome of the transgenic animal produces a product containing both AAVS1 and vector sequences. The amplified PCR product, provided the viral vector integrated into the AAVS 1 site of the animal, includes vector sequence.

### Example 15

### FVIII Transgenic Mouse Harboring the Human AAVS1 Integration Sequence and a Transgenic Mouse Tolerized to Human FVIII

### FVIII Transgenic Mouse Harboring the Human AAVS1 Integration Sequence:

An AAV/ITR-Rep vector comprising either the neo or GFP reporter gene (GT9003 and GT9012, respectively) was transfected into human 293 cells. Extracts of these cells were then assayed by Southern blot for site-specific integration of the vector at the endogenous AAVS1 site. Integration at AAVS1 was observed at a frequency of 50% in samples obtained following transfection for either the neo or GFP version of the AAV-ITR/Rep vector (GT9003 or GT9012, respectively). These results indicate that the AAV ITRs and Rep coding sequences are sufficient to direct high-efficiency, site-specific integration at AAVS1 (56). To demonstrate that the minimal Ad-hFVIII vector containing the AAV-ITR/Rep integration element is targeted to AAVS1 in vivo, integrates in a site-specific manner and maintains long-term expression of hFVIII, an animal model system was developed.

Disclosed herein is a transgenic mouse harboring the human AAVS1 integration site within its genome. The transgenic mouse is generated using embryonic stem cell manipulation technology (43). An expression vector comprising the entire 8.2 kb human AAVS1 sequence and neo (or Neo) selection marker is constructed. The AAVS1/Neo vector is transfected into totipotent mouse embryonic stem (ES) cells to obtain Neo^{R} AAVS1⁺ ES cell clones that are subsequently microinjected into mouse blastocyst-stage embryos and implanted into the uterus of a foster mother. Following implantation, the AAVS1⁺ ES cells resume normal embryonic development and contribute their genetic information (including the human AAVS1 sequence) to the developing embryo. Chimeric (transgenic) progeny are identified by the presence of ES cell-derived agouti-brown coat color. Chimeric founders are then bred with wild-type C57BL/6 mice to obtain germline transmission of the transgene. F1 heterozygotes are bred to obtain a homozygous mouse line which has stably incorporated the human AAVS1 integration sequence into its genome. This mouse model is then injected, via the tail vein or portal vein, with the AAV-ITR/miniAd-FVIII vector to assess in vivo transduction efficiency, integration at the human AAVS1 sequence, and duration of transgene expression.

### A Transgenic Mouse Tolerized to Human FVIII:

Also provided by the present invention is a FVIII-tolerized mouse model system. In the past, FVIII tolerization has been achieved by transient injection of FVIII into neo-natal mice (44). The present invention comprises a mouse having the human FVIII gene under the control of a promoter that functions in a developmental stage-specific manner. Such a promoter may include but is not limited to that of the α-fetoprotein gene or the embryonic globin gene, epsilon. The α-fetoprotein promoter is an example of an early developmental stage-specific promoter that is inactive in the mature animal (45). The embryonic globin gene, epsilon, is another example of a developmentally regulated gene that may be utilized in the present invention. Under the control of the α-fetoprotein promoter, gene expression is limited to the liver and is dependent upon liver specific transcription factors for activation (46, 47). As the normal site of FVIII expression and the preferred target organ for FVIII gene delivery is the liver, a liver specific promoter element that is also developmentally regulated would be preferred. The α-fetoprotein promoter (AFP) meets both of these criteria. The α-fetoprotein promoter does not function in undifferentiated ES cells but is induced during differentiation (48); as such, it may be utilized to control hFVIII expression in transgenic mice.

Described herein is a transgenic mouse that has been tolerized to the xenogenic human FVIII protein. The transgenic mouse may be utilized to test delivery of human FVIII in vivo using adenoviral or AAV vector systems, or using FVIII-secreting cells in an immunoisolation device. In this system, human FVIII is expressed embryonically in the developing transgenic mouse under control of the AFP promoter. HFVIII, then, is seen as “self” by the mouse and tolerance occurs. As the mouse is tolerized to hFVIII, an immune reaction does not occur toward the xenogenic human FVIII transgene product when it is delivered by the therapeutic vector (i.e., the AAV-minimal Ad-FVIII). In this manner, an accurate assessment of the antigenicity of the viral vector backbone, as well as a reliable measurement of the duration of gene expression in vivo. Also, as the AFP-FVIII transgene is expressed only during embryogenesis (and not after the animal matures), accurate levels of hFVIII delivered to the liver of the mature transgenic mouse.

### Example 16

### Construction of AFP-FVIII-Neo Vector

To generate a vector capable of driving expression of human Factor VIII in a temporally-regulated, embryo-specific fashion in a cell in order to achieve in utero tolerization of a transgenic mice, the mouse α-fetoprotein (AFP) promoter was utilized. The plasmid GT2057 comprises a 7.5 kb AFP promoter sequence originally characterized by Urano et al. (33). A 7.2 kb Not I fragment, containing the full-length human Factor VIII gene, was isolated from pCMV-FVIII (GT2051) and cloned into GT2057 behind the AFP promoter at the Not I site. The cassette containing the AFP promoter and the hFVIII gene was subsequently cloned as an Aat II/Sal I fragment into the Neo expression vector, pGKNeo, at Aat II/Xho I. The resultant 20.2 kb vector, mAFP-hFVIII-pGKNeo, harbors the HFVIII gene under control of an embryonic promoter (AFP), and has a Neo expression cassette for selection in mammalian cells.

### Example 17

### Generation and Characterization of AFP-FVIII (+) ES Cell Clones

In order to generate a mouse embryonic stem (ES) cell which contains the AFP-hFVIII sequence, for use in the production of transgenic mice, the mAFP-hFVIII-pGKNeo vector was stably transfected into mouse ES cells. 20 ug of AFP-FVIII-Neo DNA was linearized with Aat II and electroporated into ES cells (975uFd, 252v.) using a BioRad Gene Pulser. Following electroporation, ES cells were propagated on embryonic fibroblast feeder layers in 250 ug/ml G418 to select for Neo^{R} clones. 48 NeoR clones were picked, expanded, and analyzed for functional Factor VIII protein using a Coatest kit. No FVIII was detected in tissue culture supernatants from any of the 48 clones. Genomic DNA was isolated from 13 Neo^{R} ES clones and from untransfected parental ES cells, digested with Xba I, and screened by Southern blot analysis using the 7.2 kb FVIII Not I fragment from GT2051 as a probe. 11/13 transfected clones contained the expected 7.8 kb Xba I fragment, confirming the presence of the entire hFVIII sequence as well as 500 bp of the 3′ end of the AFP promoter. Analysis of four of these AFP-FVIII (+) ES clones, as well as the parental ES cell control, is shown in related patent applications (see Related Application section, supra). Such results indicate that the hFVIII transgene was present in the Neo^{R} clones.

To test whether the AFP promoter was functional in ES cells, the AFP promoter was cloned as an EcoR1/Sal I fragment into the reporter plasmid, pEGFP-1 (Clontech), to drive expression of the Green Fluorescence Protein (GFP). The pAFP-EGFP-1 plasmid was transiently transfected into both ES cells and HepG2 cells (a liver cell line known to express high levels of α-fetoprotein), and examined by direct visualization using a Nikon Diaphot broad range microscope with a FITC filter after 24 hours for green fluorescent cells. GFP expression was detected in the HepG2 cell line but not in mouse ES cells (data not shown), confirming that the AFP promoter does not function in undifferentiated ES cells, but on in a differentiated liver cell line. These results are in agreement with those of Vogt et. al. (48), which showed that the AFP promoter was silent in undifferentiated F9 embryonic stem cells, and activated when induced to differentiate following treatment with retinoic acid. To confirm that the mAFP-hFVIII-pGKNeo vector was functional in a cell line where expression would be expected, the mAFP-hFVIII-pGKNeo vector was transfected into HepG2 cells and 10×-concentrated supernatants were analyzed for hFVIII protein expression using the chromogenic Coatest FVIII assay (Pharmacia, Piscataway, N.J.). Human FVIII was detected at the level of 3.0 ng/10⁶ cells/24 hrs., confirming that the AFP-FVIII construct was functional and that the tissue-specific and developmental-specific expression pattern of the 7.5 kb AFP promoter/enhancer element was preserved. Having demonstrated that the AFP-FVIII vector was functioning properly, one of the AFP-FVIII (+) ES clones, clone #22, was chosen, based on its un-differentiated growth phenotype, to use for blastocyst microinjection experiments.

### Example 18

### Production of hFVIII-tolerized Transgenic Mice

This example describes transgenic mice that have been tolerized in utero to the xenogeneic human Factor VIII protein. This transgenic mouse is used to test delivery of human FVIII in vivo using adenoviral or AAV vector systems, or using FVIII-secreting cells in the TheraCyte immunoisolation device such as that described in U.S. Pat. Nos. 5,314,417; 5,344,454; 5,421923; 5,453,278; 5,545,223; or 5,569,462. As hFVIII is expressed embryonically in the developing transgenic mouse under the control of the AFP promoter, hFVIII as delivered by the therapeutic vector (i.e. the AAV-miniAd-hFVIII) is recognized by the immune system of the mouse to a “self” antigen. As such, tolerance to the hFVIII protein results. As the transgenic mouse is tolerized to hFVIII, an immune reaction to the “xenogeneic” human FVIII protein will not occur, and an accurate assessment of antigenicity of the viral vector backbone and a realistic measurement of the duration of gene expression in vivo may be determined. Also, as the AFP-FVIII transgene is expressed mainly during embryogenesis, the amount of hFVIII protein expressed by the liver as a result of transduction by the vector in mature transgenic mice may be accurately quantitated.

The scheme for the generation of hFVIII-tolerized transgenic mice is outlined in related patent applications (see Related Applications section, supra). Briefly, ES cells from the AFP-FVIII (+) ES clone #22, were microinjected into C57BL/6 blastocysts, and implanted into the uterus of foster mothers. To date, four chimeric progeny have been produced using ES clone #22. They are mated with wild-type C57BL/6 mice to test for germline transmission, and germline founders bred to obtain a homozygous AFP-hFVIII transgenic mouse line. Since the chimeric progeny, by definition, have mosaic expression of the AFP-hFVIII transgene in all of their tissues, they are also used directly for in vivo gene delivery experiments, without having to wait for production of a homozygous line. Transgenic animals produced by this scheme, are initially challenged by an injection of hFVIII protein, bled, and screened for antibodies to the human protein, to ensure tolerization to hFVIII. The AFP-hFVIII-tolerized transgenic mice will also be tested for “leaky” expression of hFVIII in the adult animal. If a small amount of hFVIII protein is produced in adult transgenic animals, it is accurately quantitated so that it can be subtracted from the levels of HFVIII delivered by the therapeutic vector or protein delivery device. Provided the transgenic animal is tolerized to hFVIII and expresses insignificant levels of endogenous human protein, it can be used to test the efficiency of in vivo delivery of hFVIII, the duration of gene expression, tissue distribution, and immune reactions to elements of the delivery system, other than the transgene (i.e., vector backbone, viral coat proteins) may be analyzed. Other parameters may also be tested using the transgenic animal.

### Example 19

### Second Generation Transgenic Animal Models

a. Breeding of AFP-HFVIII Tolerized Mouse With A Mouse FVIII Knockout:

A transgenic mouse strain with a targeted disruption (gene knockout) of the mouse Factor VIII gene has been obtained through a non-exclusive, restricted-use license agreement with John Hopkins University and The University of Pennsylvania. This mouse line has severe MFVIII deficiency and thus is a useful model for hemophilia A (51). By crossing our transgenic mouse tolerized to human Factor VIII with a mouse that is totally deficient for mouse Factor VIII, it is possible to produce a “clean” model for testing in vivo delivery of hFVIII. In the absence of mouse FVIII protein having the potential to cross-react with hFVIII, an accurate quantitation of hFVIII. In addition, this doubly-transgenic mouse provides a useful model for the phenotypic correction of hemophilia A using gene therapy.

b. A Triple Transgenic Mouse:

A further embodiment of this invention involves the crossing of all three above-mentioned transgenic animals to produce a “triple-transgenic” mouse model. The mouse described in the previous section, which is tolerized to human FVIII and deficient in mouse FVIII, is cross bred with the AAVS1 transgenic mouse line. This triple transgenic mouse model is preferredly suited for testing all aspects of our AAV-miniAd-hFVIII vector system including: site-specific integration at AAVS1 via the AAV ITR/Rep integration system; delivery and long-term expression of the human FVIII transgene without immune reaction to the tolerized transgene; accurate quantitation of delivered hFVIII due to a lack of adult expression of human FVIII as well as a lack of cross-reacting mouse FVIII protein; and, finally, genetic and phenotypic correction of severe FVIII deficiency (hemophilia A).

c. A Transgenic Mouse Tolerized to Green Fluorescence Protein (GFP):

It is convenient to incorporate the GFP expression cassette into the various virus vectors as a reporter gene in new minimal Ad vectors, AAV vectors, and novel versions of helper virus are developed. Viral infection, expansion, helper complementation and in vivo delivery to target cells is easily followed by visual detection of green fluorescence. It has been shown that immune responses to transgene-encoded proteins can negatively impact the stability of gene expression following injection of adenovirus vectors (30). In order to eliminate immune responses to the GFP transgene incorporated into the vector, which could shorten the duration of GFP expression after injection into mice, a GFP-tolerized transgenic mouse is developed. The AFP-EGFP-1 vector or a similar vector comprising the Rat Insulin Promoter (RIP) for pancreas-specific expression of GFP could be used for this purpose.

A RIP-EGFP-1 vector was used to transfect mouse ES cells in order to develop a stable, Neo^{R} RIP-GFP ES cell line [RIP-GFP(+) ES]. RIP-GFP(+)ES cells are utilized to generate a GFP-tolerized transgenic mouse, in a manner identical to that described for the generation of an AFP-hFVIII tolerized mouse model, substituting the RIP-EGFP-1 vector for the AFP-hFVIII vector. The RIP-GFP tolerized mouse thus produced provides a useful research tool for the development of novel adenovirus vectors or other delivery systems that utilize the GFP transgene as a reporter.

All of the above-described transgenic animal models, including the AAVS 1 transgenic mouse provided in Example 12, the hFVIII-tolerized transgenic mouse of Example 15, and the GFP-tolerized transgenic mouse of Example 19C, may be alternatively generated by direct DNA injection of the transgene (pAAVS1-Neo, mAFP-hFVIII-pGKNeo and RIP-EGFP-1, respectively). This is accomplished by injection of the transgene into the male pro-nucleus of mouse single cell ova to produce transgenic mice, as an alternative to using the ES cell technology described above. To one skilled in the art, this is an obvious alternative method for producing a transgenic mouse. The present inventions, therefore, may be produced by either of the methods discussed in this application (57-61).

### Example 20

### Episomal Minimal Ad Vectors

As another approach to provide elements that will allow long-term expression of the transgene delivered by the mini-viral vector, the present invention provides designs for a site-specific recombinase-based system that permits excision of an auto-replicative episome from the mini-viral sequences upon infection of target cells.

Site-specific recombinases have been extensively used to manipulate DNA. Site-specific recombinases catalyze precise recombination between two appropriate target sequences, cleaving DNA at a specific site and ligating it to the cleaved DNA of a second site (reviewed in ref. 111). Several systems have been identified and characterized such as the cre/loxP system from bacteriophage P1 (111) or FLP/FRT from yeast (112). The recognition sites (loxP and FRT) for both recombinases (cre and FLP) share a common structure: they have two inverted repeat elements (recombinase binding site) flanking a central core region (site of crossing-over). The orientation of the target sites (as defined by the core region) is responsible for the final outcome: recombination between two parallel sites on the same molecule results in excision of intervening sequences generating two molecules, each one with a target site. Recombination between two antiparallel sites results in inversion of the intervening sequence. Recombination between two parallel sites in different molecules results in the integration of sequences flanked by target sites. Since excision is an intramolecular event, it is favored over integration.

In the design of the present invention, recombinases will be used to excise sequences having an eukaryotic origin of replication (ori). Mammalian ori sequences and binding factors have not been characterized to date. However, some viral ori sequences and viral proteins required for initiation of replication have been characterized and incorporated in plasmid vectors, some examples of which including but not limited to SV40 ori/T-Ag from simian virus 40 (113) and oriP/EBNA-1 from Epstein-Barr virus (114). These elements have allowed the generation of plasmids that replicate autonomously in eukaryotic cells and are stably maintained upon selective pressure. Plasmids containing oriP and expressing EBNA-1 protein replicate once per cell cycle (115, 116) and are lost when selective pressure is removed from cells in culture. However, there is no in vivo data about the stability of episomal plasmids in nondividing cells, such as hepatocytes. One should expect that in nondividing cells (i.e., differentiated cells) and without selection, an episome could remain stable for a long period of time. It is believed by the inventors of the present invention that the incorporation of ori sequences in the mini-viral DNA will permit a extended expression of the transgene in nondividing cells.

The episomal minivirus elements include, but are not limited to:

a) Recombinase expression cassette: recombinase must be expressed only in target cells, because inappropriate expression in the cells used to generate the virus will promote the excision of the sequences contained between two recombination sites. For this reason, expression is tightly controlled by either adding binding sequences for transcriptional repressors upstream of the promoter (for instance, tetO ) or through the use of tissue-specific promoters (e.g., albumin promoter, factor VIII promoter, and the like).

b) Origin of replication (ori): must include the sequence to initiate or begin replication of DNA and any other element required for replication (ex: DNA binding protein recognizing origin sequences).

c) Transgene: may be any therapeutic or reporter gene flanked by a recombination site (5′) and a polyA signal sequence (3′). It will be expressed only in target cells upon circularization of the DNA.

d) Recombinase target sites: two sites are necessary in parallel orientation, one being placed between the promoter and the recombinase cDNA and the other upstream of the therapeutic gene cDNA.

e) Adenovirus ITRs: necessary for replication and packaging of the minivirus.

f) Stuffer DNA sequence: if necessary to increase the size of the minivirus up to a packageable length. The stuffer DNA sequence may be any DNA fragment of any length.

Under this design, the recombinase is not expressed while amplifying the minivirus. When the mini-viral vector is delivered to target cells, the promoter is functional, recombinase is expressed and the sequences contained between two recombinase target sites are excised and circularized. The recombinase promoter turns into the transgene promoter and the presence of the origin of replication allows stable maintenance of the plasmid, therefore assuring stable expression of the transgene.

### Example 21

### Design of The Minimal Ad for Treatment of Cancer

Currently, one of the most effective approaches to the treatment of cancer using gene therapy is to alter the tumor-host relationship and facilitate the recognition and destruction of malignant cells using the immune system. In the tumor bearing individual, the lack of an effective immune response may be due in part to either weak tumor cell immunogenicity, lack of immune co-stimulation, or a tumor-specific immunosuppressive environment. Cytokine-mediated gene transfer of tumor cells offers one strategy to augment the immune system to mount a more effective antitumor response (117). In recent years a number of cytokine genes have been isolated, cloned and characterized. Systemic administration of certain of these immunomodulators, such as IL-2, has resulted in a proportion of antitumor responses. However, toxicities have accompanied the use of many of these biologics owing to the high concentrations needed to generate clinical effects. The combination of significant undesired effects and marginal therapeutic outcomes from systemic administration has stimulated efforts to genetically engineer tumor cells to produce the cytokines themselves (118).

In animal models, gene modified tumor cells have been used as vaccines to stimulate antitumor responses (117, 119). The appeal of tumor directed cytokine gene transfer is that the cytokine, produced locally, is immunologically more efficient and does not cause systemic toxicity. Tumor antigens expressed on neoplastic cells presented with high local concentration of the cytokine(s), would create an immunological microenvironment impossible to reproduce with exogenous cytokine administration. This immunological microenvironment created by the cytokine producing tumor cells has been efficient in generating cytotoxic T lymphocytes. In a number of different animal models, cytokine producing tumor cells have been shown to be effective in decreasing the tumorgenicity and increasing the expression of immunologically important molecules (117, 119). The initial antitumor rejection appears to be accompanied by a nonspecific inflammatory response. However, rejection of cytokine secreting tumor cells has in most instances led to the generation of systemic, tumor specific immunity that is T cell dependent.

A requirement for preexisting tumor immunogenicity has not been established for most gene transfer models; however many well-characterized tumor cell lines are highly differentiated and immunogenic. In some systems, nonimmunogenic tumors have been shown to generate immunity after cytokine gene transfer. Furthermore, most tumor directed gene transfer models do not lend themselves to investigations in which the host is treated in the presence of an existing tumor burden because the rapid growth of these malignancies provides little time for immunotherapeutic intervention (119).

Recent research has demonstrated that the reduction of TGFβ secretion by tumor cells may be a significant approach to cancer gene therapy (120, 121). In one set of experiments Fakhrai et al., used antisense to TGFβ to inhibit the expression of that cytokine in a rat gliosarcoma cell line. Immunization of tumor-bearing rats with the antisense modified tumor cells resulted in significant survival of animals compared to animal's immunization with tumor cells modified with control vectors. Using a different approach Isaka et al., was able reduce the amount fibrotic disease in rats, by transfecting skeletal muscle with a cDNA encoding decorin. Decorin is a small proteoglycan that inhibits the expression of TGFβ. Thus, two different approaches to inhibit TGFb expression has shown efficacy in two different models of cancer or pre-cancer.

In addition, new evidence demonstrates that co-stimulation of T cells by B7 has both a positive and negative effect on T cell activation (122). Other co-stimulatory molecules for T cells such as ICAM-I, LFA-3 and VCAM-I have also been implicated in the induction of appropriate anti-tumor responses (123). A general consensus among those skilled in the art is that the most important of these co-stimulatory signals is provided by the interaction of CD28 on T cells with its primary ligands B7-1 (CD80) and B7-2 (CD86) on the surface of antigen presenting cells (124). In a variety of model systems tumor cells transfected with the B7 cDNA induced potent antitumor responses against both modified and unmodified tumor cells. CTLA-4, a molecule also expressed on T cells, binds B7-1 and B7-2 with much higher affinities than CD28. Results of several studies demonstrate that CTLA-4 acts as a negative regulator of T cell responsiveness, and raises the possibility that blocking the inhibition delivered by the CTLA-4-B7 interaction might augment the T cell response to tumor cells and enhance antitumor activity. It has been demonstrated that injecting antibodies to CTLA-4 resulted in the rejection of tumors including pre-established tumors in a mouse model (124). This demonstrates the care must be used in designing gene transfer experiments such that the desired effects are not masked by other potential deleterious effects.

The genetic basis of cancer includes abnormalities in oncogenes and/or tumor suppressor genes. Both types have been the targets of cancer gene therapy. Because the cancer-related defects of tumor suppressor genes are usually mutations or deletions, the strategy in tumor suppressor gene therapy thus far developed has been gene replacement therapy, in which a wild-type tumor suppressor gene is transferred into cancer cells to restore the normal function of the defective gene or induce tumoricidal effect (124). The human tumor suppressor genes that have been cloned and characterized include Rb, Wilms tumor (WT1), and neurofibromatosis (NF1), which are involved in pediatric cancers; adenomatosis polyposis coli (APC) and deleted in colon cancer (DCC), which contribute to colorectal cancer; and p53, which is found in mutated forms in a wide range of human cancers (reviewed in ref. 125).

Recently, two major events occurred in the area of identification of new tumor suppressor genes or cancer susceptibility genes. First, two highly related members of the cyclin-dependent kinase (cdk) inhibitor family, termed p16 (major tumor suppressor 1, MTS1) and p15 (MTS2), were isolated from the chromosomal region 9p21 (126-128). Second, a strong candidate for the breast and ovarian cancer susceptibility gene BRCA 1 was identified (129). While p16 was shown to be deleted or mutated in a wide range of cancer cell lines, p15 was shown to be a potential effector of TGF-β-induced cell cycle arrest (130). Among all of those tumor suppressor genes, the p53 gene is the one that has thus far been utilized for gene therapy of cancer (131).

A current effort on gene therapy of cancer is to combine tumor suppresser gene and immunomodulation gene therapy of cancer with the introduction of other molecules such as, tumor antigens, MHC molecules, cell adhesion molecules and other immunomodulating factors. The following is a general description of two designs of anticancer super-Ad vectors.

21.1 Construction the First Version of the Anti-cancer Super-Ad Vectors:

Several combinations of immune molecules and genes may be utilized in the construction of anti-cancer super Ad vectors. The minimal Ad vectors may carry of the multiple genes that function to suppress tumor growth or induce host anticancer immune responses. This type of vectors is called anticancer super-Ad vectors. The first version of the super-Ad vector will carry four double expression cassettes for human p53 cDNA, GFP marker gene, human IL2 cDNA, human GM-CSF cDNA, human B7-1 cDNA, human IL7 cDNA and human IL12 p35 and p40 cDNA. It also contains minimum sequence of left and right Ad5 ITR and Ad5 packaging sequence (total 660 bp) and about 18 kb genomic sequence of human a-fetoprotein gene to reach over 30 kb size. Cassette 1 includes a CMV promoter, a Human p53 cDNA, an EMC-IRES, a GFP gene and a SV40 pA. Cassette 2 includes an EF promoter, a human GM-CSF cDNA, an EMC-IRES, a human IL12 cDNA and a bovine growth hormone pA. Cassette 3 comprises an SV40 promoter, human B7-1 cDNA, an EMC-IRES, human IL7 cDNA and SV40 pA. Cassette 4 includes a tk promoter, a Human IL12 p35 cDNA, an EMC-IRES, a human IL12 p40 cDNA and a bovine growth hormone pA.

21.2 Construction the Second Version of the Anti-cancer Super-Ad Vectors:

A second version of the anti-cancer super Ad vectors has a similar structure to that of the first version, including adenovirus inverted terminal repeats at both the 5′ and 3′ ends and four discrete expression cassettes. Several combinations of regulatory molecules and genes may be utilized in the construction of anti-cancer super Ad vectors. The examples described below are not in any way limiting to the types of minimal Ad vectors that may be constructed to regulate the growth of a tumor cell. Each expression cassette is flanked at the 5′ end by a unique promoter. In addition, each expression cassette incorporates two genes linked by the encephalomyocarditis virus internal ribosome entry site sequence for cap independent translation of the “distal” gene. The genes shown for this vector include cytokine genes as represented by IL-2, IL-7, and GM-CSF; a tumor suppresser gene as represented by p53; immune cell co-stimulatory molecules as represented by B7-1 and ICAM-1; and molecules that can reverse the immune suppression often associated with cancers, anti-TGFβ and SCA to CTLA-4. To increase the size of the vector so that the vector will be efficiently packaged into progeny virus, we have included a “stuffer DNA” of human alpha-fetoprotein. The stuffer DNA may include any DNA fragment of any length. The general structure of the second version of the anticancer super-Ad vectors is shown in related applications (see Related Applications, supra).

### Example 22

### Minimal Ad Vectors for Immunization to Treat or Prevent Disease or other Medical Conditions

Minimal Ad vectors may be engineered to drive expression of certain antigens or immunogens that will serve to generate immunity in the organism in which expression takes place. Minimal Ad vectors may be designed that drive expression of bacterial, fungal, parasitic, viral, receptor or ligand genes that induce an immune reaction for the treatment or prevention of disease or other medical condition. For example, a coat protein from a retrovirus such as HIV may be encoded by a minimal Ad vector. Upon infection of cells in a host with a recombinant adenoviral particle comprising such a minimal Ad vector, immunity to the HIV virus may ensue. Minimal Ad vectors may also be designed to drive expression of cancer-specific antigens. Upon infection of cells in a host with a recombinant adenoviral particle comprising a minimal Ad vector directing expression of a cancer antigen, immunity to that type of cancer may follow. Optimally, such immunity will result in widespread eradication of the primary tumor as well as other metastases and micrometastases that exist throughout the treated organism. Additionally, minimal Ad vectors may be designed that encode antigenic molecules derived from one or more parasites. Other minimal Ad vectors may be designed that encode antigenic molecules derived from receptors or ligands.

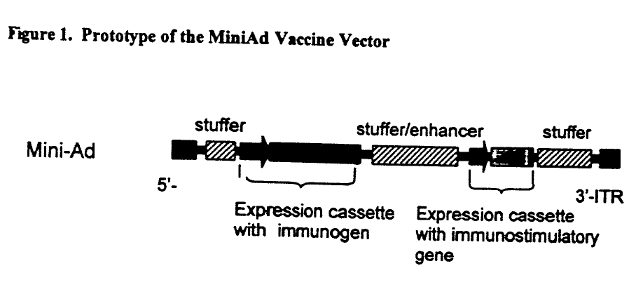

Following administration of a pharmaceutical composition comprising such minimal Ad vectors, immunity to such antigens, receptors or ligands can inhibit their function and can be useful clinically by blocking autoimmune reactions in the case of antigen receptors or for birth control by blocking the ligand beta-human chorionic gonadotropin, as examples. An exemplary minimal adenoviral vaccine vector is shown in FIG. 1.

### Minimal Ad/HIV Vectors

The HIV-1 derived immunogen HIV gag/pol/tat^{nf}/rev generates viral particles, mimicking attenuated virus vaccines without the risk of infection. Safety regarding the emergence of an infectious virus is assured by deleting the accessory genes vif, vpr, vpu and nef as well as the envelope gene env. Additionally, both HIV LTRs and the packaging signal may be deleted. Removing these accessory proteins is of further advantage since, for example, the Nef and Vpr proteins are known to cause neuronal damage (Trillo-Pazos, et al. (2000) “Recombinant nef HIV-IIIB protein is toxic to human neurons in culture” Brain Res. vol. 864, pp. 315-; Piller, et al. (1998) “Extracellular HIV-1 virus protein R causes a large inward current and cell death in cultured hippocampal neurons: implications for AIDS pathology” Proc. Natl. Acad. Sci. USA, vol. 95, pp. 4595-).

HIV gag/pol/tat^{nf}/rev encodes for the regulatory protein HIV Rev, which ensures export of mRNA into the cytoplasm. Rev is not known to be toxic. An added benefit is the fact that HIV Rev itself can elicit immune responses to help combat HIV infection (Chan, et al. (1998) “Genetic vaccination-induced immune responses to the human immunodeficiency virus protein Rev: emergence of the interleukin 2-producing helper T lymphocyte” Hum. Gene Ther., vol. 9, pp. 2187-; Blazevic, et al. (1996) “Interleukin-10 gene expression induced by HIV-1 Tat and Rev in the cells of HIV-1 infected individuals” J. Acquir. Immune Defic. Syndr. Hum. Retrovirol., vol. 13, pp. 208-). An alternative to the HIV Rev/RRE export system is provided with other lentiviral export elements such as, for example, the FIV Rev/RRE element, or export elements that do not require the expression of an additional protein such as WPRE (Pre element from Woodchuck Hepatitis Virus) (Donello, et al. (1998) “Woodchuck hepatitis virus contains a tripartite posttranscriptional regulatory element” J. Virol., vol. 72, pp. 5085-) or CTE (cytoplasma transport element from Mason Pfizer Monkey virus (Ernst, et al. (1997) “Secondary structure and mutational analysis of the Mason-Pfizer monkey virus RNA constitutive transport element” RNA, vol. 3, pp. 210-). A second regulatory protein, Tat is a transactivator and known to be a powerful immunogen (Cafaro, et al. (1999) “Control of SHIV-89.6P-infection of cynomolgus monkeys by HIV-1 Tat protein vaccine” Nat. Med., vol. 5, pp. 643-). Therefore, Tat is retained in Minimal Ad/HIV. However, it is believed that Tat protein can also synergize with inflammatory cytokines to promote angiogenesis and Kaposi's sarcoma (Barillari, et al. (1999) “Inflammatory cytokines synergize with the HIV-1 Tat protein to promote angiogenesis and Kaposi's sarcoma via induction of basic fibroblast growth factor and the alpha v beta 3 integrin” J. Immunol., vol. 163, pp. 1929-), promotion of vascular cell growth (Barillari, et al. (1999) supra) and transactivation of tumor necrosis factor-β (Brother, et al. (1996) “Block of Tat-mediated transactivation of tumor necrosis factor beta gene expression by polymeric-TAR decoys” Virology, vol. 222, pp. 252-). In a preferred embodiment, then, the minimal Ad/HIV vector expresses a nonfunctional form of Tat (Caselli, et al. (1999) “DNA immunization with HIV-1 tat mutated in the trans activation domain induces humoral and cellular immune responses against wild-type Tat” J. Immunol., vol. 162, pp. 5631-) that retains the immunogenic potential of wild-type Tat.

Cytokines (including, but not limited to, IL-3, GM-CSF, IL-12, and the like) are also useful for stimulating the immune responses and may be included in the minimal Ad/HIV vector. It is also possible to configure the vector to encode other antigens (including, but not limited to, envelope proteins of different clades of HIV) or to be equipped with the mechanisms to enhance the induction and response of the host immune system to the immunogens.

It has been shown that the HIV-derived immunogen can be presented by dendritic cells (DCs) and elicit T-cell responses (Gruber, et al. (2000) “Dendritic cells transduced by multiply deleted HIV-1 vectors exhibit normal phenotypes and functions and elicit an HIV-specific cytotoxic T-lymphocyte response in vitro” Blood, vol. 96, pp. 1327-). DCs are highly-specialized, professional antigen presenting cells (APC) which are uniquely capable of eliciting potent T cell-dependent responses. Presentation of antigen by APCs is critical for induction of an antigen-specific CTL response. DCs can generate and activate the primary immune responses and have been proposed for vaccination strategies in multiple disease settings. Transduced DCs expressing the HIV-1 immunogen HIV gag/pol/tat/rev elicit a primary antiviral cytotoxic T cell response in vitro.

### Experimental Design

As an example, the construction of three adenoviral constructs coding for the disabled HIV-1 multiprotein gag/pol/tat^{nf}/rev are described herein. Two constructs encode the HIV immunogen only (Minimal Ad/HIV and Minimal Ad/2HIV) and the third construct additionally encodes the cytokine GM-CSF (Minimal Ad/HIVc). The HIV transactivator Tat encoded by the vector is nonfunctional (tat^{nf}) but retains all known antigenic epitopes.

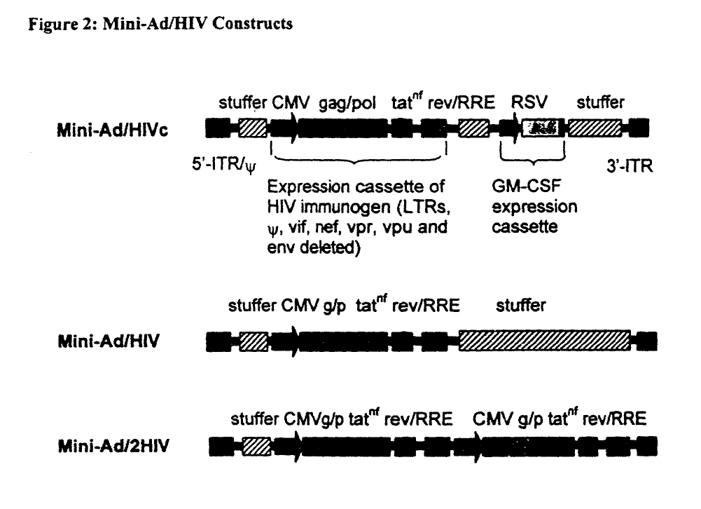

The multiprotein HIV gag/pol/tat/rev (4.6 Kb) is derived from the molecular clone HIV-1-MN (WO 97/36481) and encoded in a single expression cassette regulated by the CMV promoter (0.8 Kb) at the 5′ end and the poly A signal of the bovine growth hormone (0.23 Kb) at the 3′ end. Expression of murine GM-CSF (0.8 Kb) is regulated by the RSV promoter (0.7 Kb) at the 5′ end and the SV40 poly A signal (0.2 Kb) at the 3′ end. A schematic of exemplary adenoviral constructs is shown in FIG. 2.

The cDNA for the HIV-1gag/pol/tat/rev is derived from the previously described HIV-1 based lentiviral vector HIV-1ΔEN (Gruber, et al. (2000) “Dendritic cells transduced by multiply deleted HIV-1 vectors exhibit normal phenotypes and functions and elicit an HIV-specific cytotoxic T-lymphocyte response in vitro” Blood, vol. 96, pp. 1327-). Tat is replaced by the 4.6 kb nonfunctional tat cDNA fragment and cloned into a cassette with the CMV promoter upstream and the bovine poly A signal downstream of the cDNA. The approximately 6 kb tat expression cassette is then tested for functionality in a eukaryotic expression vector before being cloned into the Minimal Ad vector pGT4163. The bovine growth hormone poly A signal polyadenylation sequences are derived from plasmid pCDNA3 (Invitrogen Corp., San Diego, Calif.) using standard PCR techniques. For the GM-GSF expression cassette, the RSV promoter is isolated from plasmid pOP13CAT (Stratagene, La Jolla, Calif.) and cloned upstream of the murine GM-CSF cDNA, which is isolated from a murine cDNA library (Stratagene, La Jolla, Calif.) using PCR methods. The SV40 polyA site is isolated from the pOP13CAT plasmid and cloned downstream of the GM-CSF cDNA immediately before the 3′ DNA stuffer region (“stuffer” contemplates endogenous or exogenous nucleic acid sequences to maintain the length of the minimal adenovirus for packaging). The resulting 2 Kb expression cassette is then cloned downstream of the HIV-1 expression cassette. A minimum of at least 28 Kb is required to efficiently package a functional minimal Ad vector. Genomic sequences from AFP, albumin and β-actin are utilized as “DNA stuffer” sequences between the packaging signal and the HIV-1 cassette, between the HIV-1 and the GM-CSF cassette and the GM-CSF cassette and the 3′ ITR (see FIG. 2). It is generally known that humans generate immune responses to the gag, pol and nef antigens and minimal adenoviral vectors (e.g., MAXIMUM-Ad™) comprising such antigens are desirable.

The adenoviral vector constructs are analyzed for expression of the HIV-1 immunogen as well as the cytokine. The constructs are transiently transfected into 293 cells using standard CaPO₄ transfection methods. At day 2- day 5 after transfection, the cell supernatant is analyzed for the presence of the HIV capsid protein p24 by ELISA (Abbott Laboratories, Chicago, Ill.). In addition, correct processing of the HIV multiprotein is verified using an anti-p24 Western blot. To complete this assay, the minimal Ad/HIV viral particles are concentrated from the supernatant by pelleting at 25,000 g for 90 minutes at 4° C. The presence of the murine GM-CSF will be detected in the supernatant by ELISA (R&D Systems, Minneapolis, Minn.).

Prior to in vivo animal experiments with minimal Ad/HIV vector preparations, transduction efficacy and viability of cell lines and DC transduced with Minimal Ad/HIV will be assessed. Expression of HIV-1 proteins among transduced DC will be determined to ascertain whether expression alters the phenotype and function of the DC. In addition, we will examine expression in a murine fibroblast cell line (NIH 3T3) and other relevant cells such as spleen- and bone-marrow-derived DC. At various time points after transduction (dl-d14), viability of the cells is determined by trypan exclusion method and the kinetics of viability following expression of HIV proteins (HIVgag/pol/tat^{nf}/rev) observed. The expression of the capsid protein p24 in cell culture supernatants is determined by standard ELISA (R&D Systems).

Once efficient transduction and stable expression of viral proteins are achieved with fibroblasts, transduction of dendritic cells is accomplished. DC are derived by culturing bone marrow cells of C57B1/6 mice with GM-CSF and IL-4 for 7 days and/or directly from splenocytes using dendritic cell enrichment columns (Stem Cell Technologies). These in vitro derived DC may be contaminated with macrophages. In that case, the cells are sorted to isolate the CD11c+ population, thus enriching for DC. In vitro derived DC are then infected with minimal Ad/HIV. Following infection on days 1 through 7, viability (by trypan blue exclusion) of transduced DC is compared to that of untransduced DC. The kinetics of expression of p24 (by ELISA) secreted by Minimal Ad/HIV transduced DC is monitored.

To assess the functional status of Minimal Ad/HIV-transduced DC, the activation status of DC and the production of cytokines by activated DC is monitored. To assess the activation status of DC, the kinetics (dl-d7 after viral transduction) of upregulation of cell surface markers, Class I and II, B7. 1 and B7.2, ICAM and CD40 is determined and compared to untransduced DC. Production of cytokines (IL-1α and β, TNFα, IL-6, IL-12 (p35 and p40) by activated DC using ELISA and/or RNAse protection assays.

The ability of Minimal Ad/HIV-transduced DC to function as APC (i.e., to present antigen and activate both CD8 and CD4 T cells) is then ascertained. One to ten ×10⁵ DC transduced with minimal Ad/HIV are injected i.v., into syngeneic (C57BL/6) mice. After 7-10 days of priming, CD8 T cells are prepared (purified using Stem Cell Columns) from DC primed mice and restimulated with Minimal Ad/HIV-transduced DC for 5 days to expand HIV-specific CTL. HIV-specific cytotoxicity is determined by chromium release assay, using fibroblasts transduced/infected with minimal Ad/HIV, FIV or HIV vectors as targets.

It is then determined whether activated DCs function as APCs for naive CD4 T cells using similar protocols as described above. Briefly, CD4 T cells prepared (using stem cell columns for enrichment of CD4 T cells from minimal Ad/HIV transduced DC- primed mice are incubated with minimal Ad/HIV-transduced DC for 48 h. Following incubation, proliferation of CD4 T cells is determined by the uptake of ³H- thymidine. IL-2 and IFNγ secretion of CD4 T cells is assayed by bioassay and/or ELISA.

Mice are then directly immunized intranasally, intramuscularly, intraparitoneally, or subcutaneously with the minimal Ad/HIV vector, and the induction of cellular and humoral immune responses evaluated in both mucosal and systemic immune compartments. The impact of boosting as well as the maintenance of immunological memory is also determined. In addition, we will assess and compare the levels of protection afforded by the minimal Ad/HIV vaccines using a challenge model incorporating a vaccinia vector encoding HIV genes.

Primary and memory CTL responses are evaluated in the spleen, draining lymph nodes and genital tissues of immunized mice. CTL are functionally evaluated in a chromium release assay by expanding splenocytes in vitro for 5-7 days using minimal Ad/HIV transfected cells. Splenocytes may then be incubated with ⁵¹Cr labeled -, gag peptide pulsed targets and targets infected with Vacc-gag in a chromium release assay.

In addition to a functional evaluation, antigen specific CTL will be quantitatively enumerated in an IFN-γ ELISPOT assay. Splenocytes are serially diluted and cultured in the presence or absence of the HIV CTL gag epitope on nitrocellulose plates coated with anti-IFN-γ. Spot forming cells (SFC) secreting IFN-γ in response to peptide stimulation and representing HIV-specific CTL are then visually enumerated. ELISPOT analysis will also be used for quantification of IL-4 and IL-2 expressing lymphocytes. Comparisons of the total numbers of antigen specific IFN-γ, IL-4 and IL-2 producing cells allows quantitative comparisons between groups. In addition, relative numbers of cytokine producing CTL indicate the phenotype of the immune response.

Specific CTL are further quantitated and characterized by flow cytometry (FACS staining). HIV-specific CTL may be quantitated by stimulating with HIV gag peptides and staining for intracellular IFN-γ production. In conjunction with IFN-γ, other characteristics of the CTL may be evaluated such as FasL, perforin expression, or the presence of costimulation molecules or adhesion molecules as well as other cytokines.

In addition to evaluating responses in the spleen, initial CTL responses may be visualized in the mediastinal/cervical lymph nodes that drain the respiratory tract following intranasal immunization. Responses observed in the draining lymph nodes indicate the state of development of mucosal CTL, an important component of a mucosal vaccine. Five days following intranasal immunization, lymphocytes are isolated from the draining lymph nodes and cultured for 3 days in vitro. HIV-specific lymphocytes are evaluated in a chromium release assay, or for IFN-γ production by ELISPOT or FACS analysis.

In addition to the primary mucosal immune response, the CTL memory response may be evaluated in the local draining lymph nodes. The presence of recall or memory CTL responses locally in the genital tract or other mucosal tissues has been shown to be essential for long term protection from mucosal challenge. To evaluate the memory response in mucosal tissues, the CTL from the local draining lymph nodes can be examined 2-3 days following intravaginal challenge. At this early time post infection, the immune response is still undetectable in naive animals, however, in vaccinated mice that have the capacity to mount mucosal CTL responses there is a substantial CTL responses that develops. Thus, by infecting mice intravaginally with a vaccinia virus vector expressing gag (Vacc-gag) the primary CTL response is evaluated in the draining iliac lymph nodes at day 2.

The presence of mucosal CTL is also be visualized directly in the genital tissues following intravaginal Vacc-gag challenge. To observe CTL in the genital tract the cells are isolated by digestion of the genital tissues 2 to 3 days following challenge. CTL specific for gag will be evaluated ex vivo by IFN-γ production in an ELISPOT assay and by FACS staining as described above.

Helper responses are evaluated and compared in the spleen or draining lymph nodes following Minimal Ad/HIV immunization. Splenocytes are cultured with tat and gag protein (p17, p24) and proliferation measured by incorporation of thymidine. In addition, IL-2 production are evaluated as a measure of the magnitude of the response. Other cytokines such as IFN-γ and IL-4 produced during this proliferative response are also be assessed as an indication of the phenotype of the response. In addition, the lymphocytes isolated during the primary or memory response from the lymph nodes draining the respiratory and genital tract are evaluated in a similar manner.

To assess whether Minimal Ad/HIV immunized mice are able to resist mucosal infection, mice will be challenged intravaginally with an HIV-gag expressing vaccinia virus vector. This vector is replication competent and encodes for gag proteins but does not incorporate these proteins in its membrane. As a result, protection or resistance to a Vacc-gag challenge will be mediated by a cellular immune response. To evaluate the level of protection following intravaginal challenge, vaccinia viral titres will be determined daily in the ovaries where the virus preferentially replicates.

### MiniAd-HPV Vector

Similar to the features of the MiniAd-HIV vector, the MiniAd-Human Papilloma Virus (HPV) vectors are designed to deliver HPV immunogens with or without immune enhancing genes. The immunogens of HPV are the early region genes 6 and 7 (E6 and E7) and the later genes 1 and 2 (L1 and L2). It was known that E6 and E7 are critical transactivators for HPV to establish productive infection in host cells. The modified E6 and E7 gene (E6^{d} and E7^{d}) products delivered by the vector may play an immunogenic function and induce anti-HPV responses, both humoral and cellular. The L1 and L2 are structural proteins and known antigens of HPV. Therefore, the L1 and L2 genes are included in the MiniAd-HPV vector.

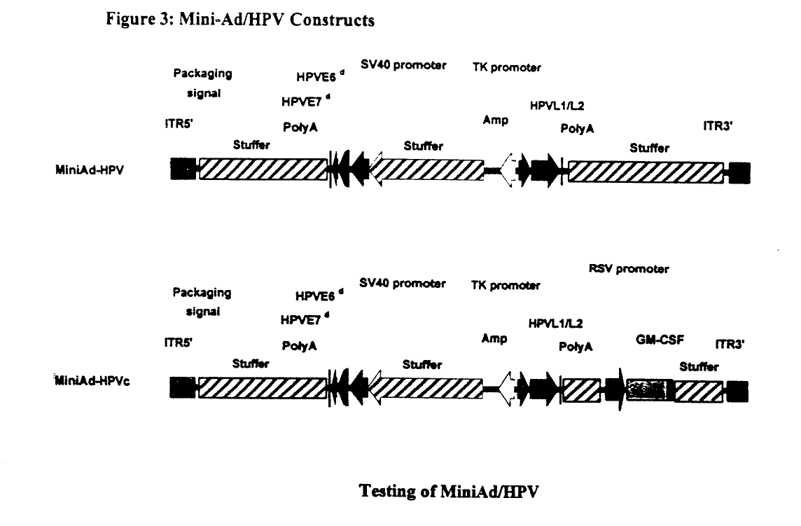

The MiniAd vector has the capacity to incorporate other genes or elements to enhance vector delivery, gene expression, vector genomic stability, and augmentation of host immune responses to the viral immunogens. The various orientations of suitable enhancer and stuffer elements are shown in FIG. 3.

Two examples of such MiniAd-HPV vectors are described herein (see FIG. 3). Each vector contains the HPV genes L1, L2, mutated E6 and E7. In one embodiment, the vector additionally contains the cytokine gene GM-CSF. The wild type L1 and L2 genes in their natural sequence are incorporated into a single expression cassette in which the TK promoter and the poly-A signal of the bovine growth hormone (BGH) gene flanking L1 and L2. To eliminate the transforming effect and to enhance the specific cellular and humoral immune response, E6 and E7 genes may be mutated using PCR. E6 and E7 are clustered using the internal ribosomal entry site (IRES) and constructed into an expression cassette contains SV40 promoter/enhancer and poly-A signal sequence. In one embodiment, the GM-CSF cDNA under the transcriptional control of the Rous Sarcoma Virus (RSV) promoter is included. DNA fragments from human genes such as albumin, alpha-fetoprotein, beta-actin, or other tissue-specific enhancer sequences may also be incorporated into the vectors to enhance the packaging efficiency and stability of the vector as well as expression of the genes encoded thereon. Exemplary embodiments of such vectors are illustrated in FIG. 3.

The MiniAd-HPV vector may be initially examined in vitro to determine whether infected cells produce E6, E7, L1, L2 and cytokine (when incorporated) gene products. Next, the induction of cellular immune responses to E6 and E7 and humoral and cellular immune responses to L1 and L2 may be examined in vivo. Finally, protection from infection by HPV may be evaluated in an animal model. The development of antibodies to L1 and L2 following immunization with MiniAd/HPV vaccine will depend on the expression of these antigens in vivo and also their formation into virus-like particles (VLPs). Humoral immunity may be evaluated in mucosal compartments as well as in serum following intranasal (IN), intramuscular (IM), intraparitoneal (IP), or subcutaneous (SC) immunization. Serum samples may be taken weekly following immunization and evaluated for IgG and IgA against the L1 protein in a capture ELISA. In addition, the subclasses of IgG specific for L1 may be evaluated (to assess the phenotype of humoral the response). Vaginal as well as lung/nasal washes may be taken at 2 weeks following immunization and evaluated for IgA and IgG content as may be done for serum above (IgG subclasses can also be determined). In addition, daily intravaginal washes may be taken and the stage of the estrous cycle may be determined by analysis of the cells present in the smear. Antibody levels may then be evaluated with respect to the stage of the estrous cycle (IgA is relatively high at estrus, and conversely, IgG is higher at diestrus). The long-term presence of VLP or L1/2-specific antibodies in the genital tract may also be determined.

The neutralizing capacity of antibodies from the serum and mucosal washes may be determined using an infectious pseudotype neutralization assay (Balmelli, et al. (1998) “Nasal immunization of mice with human papillomavirus type 16 virus-like particles elicits neutralizing antibodies in mucosal secretions” J. Virol., vol. 72, pp. 8220-; Roden, et al. (1996) “In vitro generation and type-specific neutralization of a human papillomavirus type 16 virion pseudotype” J. Virol., vol. 70, pp. 5875-). In this assay, infectious pseudotyped virions consisting of the HPV16 capsid, comprising L1 and L2, and containing the bovine papilloma virus (BPV) genome, are generated. The neutralizing capacity of mucosal and serum samples may be evaluated by quantitating the reduction in the induction of transformed foci in monolayers of mouse C127 cells.

Mucosal tissues (genital tract) may be isolated and digested 2 weeks following immunization and examined for HPV-specific B cells by ELISPOT analysis. In addition, specific B cell memory responses may be evaluated in the genital tract following intravaginal challenge with the pseudotyped virus or a vaccinia virus expressing L1 antigens. This will demonstrate the local (or mucosal-specific) component of the antibody response observed in the genital tract.

CTL responses may be evaluated in the spleen, draining lymph nodes and genital tissues of immunized mice. CTL from the spleen in at least 3 ways. First, CTL may be functionally evaluated in a chromium release assay by expanding CTL in vitro using peptide (i.e., amino acids 49-57 from E7 for H-2^{b} mice) pulsed cells or cell lines expressing HPV genes (i.e., E6/7, L1/2). Splenocytes may then be purified and cultured for 5-7 days with peptide before being incubated with ⁵¹Cr labeled E7-expressing cell lines (TC-1) or peptide pulsed targets (B16F1).

CTL may also be enumerated in an ELISPOT assay to determine IFN-γ production levels. Splenocytes are first serially diluted and cultured in the presence or absence of peptide on nitrocellulose plates coated with anti-IFN-γ. Spot-forming cells (SFC) secreting IFN-γ in response to peptide stimulation are determined, and the E7-specific CTL enumerated.

CTL may also be characterized by flow cytometry (FACS staining). E7-specific CTL are quantitated by stimulating the cells with peptide (i.e., amino acids 49-57 from E7 for H-2^{b} mice) and staining for intracellular IFN-γ production. In addition, other characteristics of the CTL may be evaluated such as the expression of FasL, perforin, or cytokines, or the presence of costimulatory or adhesion molecules.

CTL from the iliac lymph nodes that drain the genital tract or mediastinal/cervical lymph nodes that drain the respiratory tract may also be evaluated using the methods described above, for example. However, the in vitro stimulation procedure is not necessary and the primary assay can be carried out 5 days following initial immunization. In addition, the memory response can be evaluated by examining the CTL from the lymph nodes two days following in vivo challenge using the pseudotyped virus or a vaccinia virus vector expressing E6/7 or L1/2. CTL from the genital tract may be evaluated without the in vitro stimulation required for splenocytes. To observe CTL in the genital tract, cells are isolated by digestion of the genital tissues. CTL from the genital tract are visualized by IFN-γ production in an ELISPOT assay or by FACS staining.

Helper responses may be evaluated in the spleen or draining lymph nodes. Splenocytes are cultured with the recombinant E6/7 or L1/2 recombinant protein or MHC II restricted peptide of E7 (amino acids 44-62) and proliferation measured by incorporation of thymidine. IL-2 production may also be evaluated as an indicator of proliferation. Other cytokines produced during this proliferative response (IFN-γ) is also assessed as an indication of the phenotype of the response. As for spleen cells, the lymph node cells draining the respiratory tract are isolated 5-7 days post-immunization and stimulated as described above.

To evaluate the level of functional immunity in immunized mice, the animals are challenged with tumors derived from E7/E6 transformed cell lines. Growth of tumors is monitored as a read out of protection (i.e., the RMA cell line on the B6 background; Shi, W. et al. (1999) “Human papillomavirus type 16 E7 DNA vaccine: mutation in the open reading frame of E7 enhances specific cytotoxic T-lymphocyte induction and antitumor activity” J. Virol. vol. 73, no. 9, pp. 7877-7881). Human papillomavirus type 16 E7 DNA vaccine: mutation in the open reading frame of E7 enhances specific cytotoxic T-lymphocyte induction and antitumor activity. (Shi, W. et al. (1999) supra). RMA cells are injected SC and tumor volume measured over time. In addition, mice harboring tumors are immunized with the vector and monitored for clearance of established tumors. Immunized mice are also challenged IV with metastatic E6/E7 expressing tumor cells (TC-1) that localize exclusively in the lungs. This model is attractive since there is a mucosal component to the localization of the tumor. Mice can also be given TC-1 cells prior to immunization with the Minimal Ad/HPV vector. This model has been used following immunization with a vaccinia vector expressing E7 and LAMP-1 and shown protection (Ji, et al. 1998. Antigen-specific immunotherapy for murine lung metastatic tumors expressing human papillomavirus type 16 E7 oncoprotein. Int J Cancer 78:41.).

Mice may also be challenged intravaginally with a recombinant vaccinia virus expressing L1 and L2. Vaccinia viral titres are then determined from the ovaries as a readout of protection (Marais, et al. 1999. A recombinant human papillomavirus (HPV) type 16 L1-vaccinia virus murine challenge model demonstrates cell-mediated immunity against HPV virus-like particles. J Gen Virol 80:2471.). This model may also be extended to challenge with recombinant vaccinia or herpes vectors expressing L1 and L2 or E6 and E7 antigens (He, et al. 2000. Viral recombinant vaccines to the E6 and E7 antigens of HPV-16. Virology 270:146).

A rabbit challenge model offers the ability to assess papilloma warts that are similar to those observed in humans. Immunized rabbits may be challenged in the skin with cottontail rabbit papilloma virus (CRPV) and monitored for papilloma formation (Sundaram, P. et al. (1998) “Intracutaneous vaccination of rabbits with the E6 gene of cottontail rabbit papillomavirus provides partial protection against virus challenge” Vaccine, vol 16, no. 6, pp. 613-623). Complete or partial protection may be observed in all animals. A recently described rabbit oral papilloma virus (ROPV) offers a mucosal model in the rabbit. This virus shares homology in L1 with CRPV (68% a.a. identity for L1). Oral and genital infection with this virus results in lesions that spontaneously regress in conjunction with the development of humoral and cellular immunity (Christensen et al. (2000) “Rabbit oral papillomavirus complete genome sequence and immunity following genital infection” Virology 269:451).

### Example 23

### Other Designs For Improvement Of The System

### Minimal Ad Vectors Having Targeting Capability

Multiple mechanisms may be utilized to target gene expression to a specific cell type or tissue. One such mechanism involves transcriptional targeting of a cell type, cell type subset or a specific tissue. Transcriptional targeting includes the use of a transcriptional regulatory unit that drives gene expression in only a certain type of cell or tissue. Such a transcriptional regulatory unit is referred to as being tissue-specific. A minimal Ad vector is designed to incorporate a tissue-specific transcriptional regulatory unit driving expression of a reporter or effector gene. In this manner, expression of the reporter or effector gene under control of the tissue-specific transcriptional regulatory unit will be detected at a higher level in those specific tissues in which the transcriptional regulatory unit is active. It may be preferable to restrict gene expression to a certain cell type or tissue. Therapeutic genes are often toxic if expressed in high amounts. Regulation of gene expression to specific tissues, then, may serve to protect the host from the adverse effects of high level gene expression of certain therapeutic genes.

A further method to direct tissue-specific gene expression would be to utilize a helper virus that encodes a cell surface protein reactive to a ligand on a cell type of interest. For instance, a helper virus may be engineered to express a ligand for a cell surface receptor. Upon packaging of the recombinant packaging-competent DNA construct of this invention, an recombinant adenoviral particle that binds to a receptor on the surface of a cell is produced. A further example would include a recombinant adenovirus that expresses an antibody or a fragment of an antibody on the surface of its viral coat. Such a recombinant virus may be produced by engineering a packaging-deficient helper virus to express an antibody or antibody fragment as a fusion or a separate protein on its viral coat. Upon infection of a cell transfected with a DNA molecule encoding an at least an adenoviral packaging sequence and at least one reporter or effector gene, recombinant adenoviral particles having an antibody or antibody fragment reactive to a cell surface molecule on a target cell are produced. In this manner, recombinant adenoviral particles will specifically bind to those cells in the host that express cell surface molecules reactive to said antibodies or antibody fragments.

### Minimal Ad Vectors For Local Immune Suppression

Certain autoimmune disorders result from the inappropriate immune reactions. One method that may be utilized to prevent, halt or slow the autoimmune reaction is to direct expression of immunomodulatory proteins at the site of such reactions. This may be accomplished by application of adenoviral particles constructed from a minimal Ad genome as demonstrated within this application. Genes encoding certain cytokines or chemokines may be expressed and such expression may result in an attenuation of the immune reponse. This attenuation in the immune response would then lead to an alleviation of the symptoms of the autoimmune reaction. A further example may include the attenuation of an allergic reaction. An antigen known to cause an allergic reaction may be encoded by a minimal Ad vector. Upon expression either low levels or extremely high levels of the antigen, driven by the minimal Ad vector delivered to a cell by a recombinant adenoviral particle, tolerance may result. Also, expression of the antigen may be directed to tissues in which expression of the antigen may induce tolerance. Such a tissue may include the developing thymus. Following desensitization, the host into which the recombinant adenoviral particle was delivered will not exhibit an allergic reaction upon interaction with that antigen. In this manner, a form of immunosuppression has been achieved by administration of the recombinant adenoviral particle carrying engineered minimal Ad DNA molecule.

3. The minimal Ad vector that hybridize with other elements: It will also be possible to utilize the minimal Ad vectors disclosed in this application to prevent or eliminate viral infection and replication within a host. Minimal Ad vectors can be designed such that viral certain genetic processes may be interfered with or eliminated. The minimal Ad vectors may be designed to express antisense nucleic acids that interfere with viral replication at the transcriptional or translational stage of infection. Interference may be promoted by the expression of antisense RNA or DNA including that which binds to messenger RNA or binds to DNA after integration of a viral genome to prevent transcription. Also, ribozymes may be designed that target certain viral transcripts for destruction. “Decoy” molecules may also be encoded by a minimal Ad vector. Such decoys may function by binding to transcription factors required for viral transcription such that the transcription factors are no longer available for binding to and driving transcription of genes required for viral gene expression and replication.

While a preferred form of the invention has been shown in the drawings and described herein, since variations in the preferred form will be apparent to those skilled in the art, the invention should not be construed as limited to the specific form shown and described, but instead is as set forth in the claims.

### REFERENCES

1. Mannuchi, P. M. 1993. Modem Treatment of Hemophilia: From the shadows towards the light. Thrombosis and Haemostasis 70: 17-23.

2. Kazazian, H. H. Jr. 1993. The molecular basis of hemophilia A and the present status of carrier and antenatal diagnosis of the disease. Thrombosis and Haemostasis 70: 600-62.

3. Furie, B, S. A. Limentani, and C. G. Rosenfield. 1994. A practical guide to the evaluation and treatment of hemophilia. Blood 84: 3-9.

4. Jones, L. K., and E. G. D. Tuddenham. 1995. Gene therapy for hemophilias. Gene Ther. 2: 699-701.

5. Toole, J. J., J. L. Knopf, J. M. Wozney, L. A. Sultzman, L. L. Buecker, D. D. Pitman, R. J. Kaufman, E. Brown, C. B. Sheoemaker, E. C. Orr, G. W. Amphlett, B. Foster, M. L. Coe, G. J. Knutson, D. N. Fass, and R. M. Hewick. 1984. Molecular cloning of a cDNA encoding human antihemophilic factor. Nature 312: 342-347.

6. Wood, W. I., D. J. Capon, C. C. Simonsen, D. L. Eaaton, J. Gitschier, B. Keyt, P. H. Seeburg, D. H. Smith, P. Hollingshead, K. Wion, E. Delwart, E. G. D Tuddenham, G. A. Vehar, and R. M. Lawn. 1984. Expression of active human factor VIII from recombinant DNA clones. Nature 312: 330-337.

7. Truett, M. A., R. Blacher, R. L. Burke, D. Caput, C. Chu, D. Dina, K. Hartog, C. H. Kuo, F. R. Masiarz, J. P. Merryweather, R. Najarian, C. Pachl, S. J. Potter, J. Puma, M. Quiroga, L. B. Rall, A Randolph, M. S. Urdea, P. Valenzuela, H. H. Dahl, J. Favalaro, J. Hansen, O. Nordfang, and M. Ezban. 1985. Characterization of the polypeptide composition of human factor VIII:C and the nucleotide sequence and expression of the human kidney cDNA. DNA 4: 333-349.

8. Toole, J. J., D. D. Pittman, E. C. Orr, P. Murtha, L. C. Wasley, and R. J. Kaufman. 1986. A large region (˜95 kDa) of human factor VIII is dispensable for in vitro procoagulant activity. Proc. Natl. Acad. Sci. U.S.A. 83: 5939-5942.

9. Koeberl, D. D., C. L. Halbert, A. Krumm and A. D. Miller. 1995. Sequences within the coding regions of clotting factor VIII and CFTR block transcriptional elongation. Human Gene Ther. 6: 469-479.

10. Hoeben, R. C., F. J. Fallaux, S. J. Cramer, D. J. M. vand den Wollenberg, H. van Ormondt, E. Briet, and A. J. van der Eb. 1995. Expression of the blood-clotting factor-VIII cDNA is repressed by a transcriptional silencer located in its coding region. Blood. 85: 2447-2454.

11. Dwarki, V. J., P. Belloni, T. Nijar, J. Smith, L. Couto, M. Rabier, S. Clift, A. Berns and L. Cohen. 1995. Gene therapy for hemophilia A: Production of therapeutic levels of human factor VIII in vivo in mice. Proc. Natl. Acad. Sci. USA 92: 1023-1027.

12. Chuah, M. K. L., T. Vandendriessche, and R. A. Mogan. 1995. Development and analysis of retroviral vectors expressing human factor VIII as a potential gene therapy for hemophilia A. Human Gene Ther. 6: 1363-1377.

13. Connelly, S., T. A. G. Smith, G. Dhir, J. M. Gardnewr, M. G. Mehaffey, K. S. Zaret, A.

McClelland and M. Kaleko. 1995. In vivo gene delivery and expression of physiological levels of functional human factor VIII in mice. Human Gene Therapy 6: 185-193.

14. Connelly, S., J. M. Gardner, A. McClelland, and M. Kaleko. 1996. High-level tissue-specific expression of functional human factor VIII in mice. Hum. Gene Ther. 7:183-195.

15. Connelly, S., J. Mount, A. Mauser, J. Gardner, M. Kaleko, A. McClelland, and C. D. Lothrop Jr. 1996. Complete short-term correction of canine hemophilia A by in vivo gene therapy. Blood 88: 3846-3853.

16. Connelly, S., J. M. Gardner, A., R. M. Lyons, A. McClelland, and M. Koleko. 1996. Sustained expression of therapeutic levels of human factor VIII in mice. Blood 87: 4671-4677.

17. Gluzman, Y. and K. Van Doren. 1983. Palindromic adenovirus type 5-simian virus 40 hybrid. J. Viol. 45: 91-103.

18. Grable, M., and P. Hearing. 1992. cis and trans requirements for the selective packaging of adenovirus type 5 DNA. J. Virol. 66: 723-731.

19. Fisher, K. J., H. Choi, J. Burda, S -J Chen, and J. M. Wilson. 1996 Recombinant adenovirus deleted of all viral genes for gene therapy of cystic fibrosis. Virology 217: 11-22.

20. Haecker, S. E., H. H. Stedman, R. J. Balice-Gordon, D. B. J. Smith, J. P. Greelish, M. A. Mitchell, A. Wells, H. L. Sweeney, and J. M. Wilson. 1996. In vivo expression of full-length human dystrophin from adenoviral vectors deleted of all viral genes. Hum. Gene Ther. 7: 1907-1904.

21. Kochanek, S., P. R. Clemens, K. Mitani, H -H Chen, S. Chan, and C. T. Caskey. 1996. A new adenoviral vector: Replacement of all viral coding sequences with 28 kb of DNA independently expressing both full-length dystrophin and β-galactosidase. Proc. Natl. Acad. Sci. USA 93: 5731-5736

22. Kotin, R., Siniscalo, M., Samulski, J., Zhu, X., Hunter, L., Laughlin, C., McLaughlin, S., Muzyczka, N., Rocchi, M., and Berns, K. I. (1990) Site-specific integration by adeno-associated virus. Proc. Natl. Acad. Sci. USA 87: 2211-2215.

23. Samulski, J., Zhu, X., Brook, J., Housman, D., Epstein, N., and Hunter, L. (1991) Targeted integration of adeno-associated virus (AAV) into human chromosome 19. EMBO J. 10: 3941-3950.

24. Shelling, A. and Smith, M. (1994) Targeted integration of transfected and infected adeno-associated virus vectors containing the neomycin resistance gene. Gene Therapy 1: 165-169.

25. Giraud, C., E. Winocour, and K. I. Berns 1994. Site-specific integration by adeno-associated virus is directed by a cellular sequence. Proc. Natl. Acad. Sci. USA 91: 10039-10043.

26. Giraud, C., E. Winocour, and K. Berns. (1995) Recombinant junctions formed by site-specific integration of adeno-associated virus into an episome. J. Virol. 69: 6917-6924.

27. Linden, R., E/Winocour, and K. Berns. (1996) The recombination signals for adeno-associated virus site-specific integration. P.N.A.S. 93: 7966-7972.

28. Urcelay, E., P. S. M. Ward, S. M. Wiener, B. Safer, and R. Kotin 1995. Asymmetric replication in vitro from a human sequence element is dependent on adeno-associated virus rep protein. J. Virol. 69: 2038-2046.

29. Halbert, C., L. Alexander, G. Wolgamot, and D. Miller. (1995) Adeno-associated virus vectors transduce primary cells much less efficiently than immortalized cells. J. of Virology 69: 1473-1479.

30. Tripathy, S., H. Black, E. Goldwasser, and J. Leiden. (1996). Immune responses to transgene-encoded proteins limit the stability of gene expression after injection of replication-defective adenovirus vectors. Nature Medicine 2: 545-550.

31. Ghosh-Choudhury, G., Y. Haj-Ahmad, and F. L. Graham. 1987. Protein IX, a minor component of the human adenovirus capsid, is essential for the packaging of full-length genomes. EMBO J. 6: 1733-1739.

32. Hayashi, Y., J. Chan, H. Nakabayashi, T. Hasimoto, and T. Tamaoki. 1992. Identification and characterization of two Enhancers of the human albumin gene. J. Biol. Chem. 267: 14580-14585.

33. Urano, Y., M. Sakai, K. Watanabe, and T. Tamaoki. 1984. Tandem arrangement of the albumin and alpha-fetoprotein genes in the human genome. Gene 32: 255-261.

34. Berns, K. I. 1996. Parvoridiae: the viruses and their replication. Fields Virology. Philadelphia, Lippincot-Raven. Third, ed. 2173- 2197.

35. Kotin, R. M., R. M. Linden, and K. I. Berns 1992. Characterization of a preferred site on human chromosome 19q for integration of adeno-associated virus DNA by non-homologous recombination. EMBO J. 11: 5071-5078.

36. Im, D. S., and N. Muzyczka 1989. Factors that bind to adeno-associated virus terminal repeats. J. Virol. 63: 3095-3104.

37. Ashktorab, H., and A. Srivastava 1989. Identification of nuclear proteins that specifically interact with adeno-associated virus type 2 inverted terminal repeat hairpin DNA. J. Virol. 63: 3034-3039.

38. Im, D. S., and N. Muzyczka 1990. The AAV origin binding protein Rep68 is an ATP-dependent site-specific endonuclease with DNA helicase activity. Cell 61: 447-457.

39. Im, D. S., and N. Muzyczka 1992. Partial purification of adeno-associated virus Rep78, Rep68, Rep52 and Rep40 and their biochemical characterization. J. Virol. 66: 1119-1128.

40. Wonderling, R. S., S. R. M. Kyöstiö, and R. A. Owens 1995. A maltose-binding protein/adeno-associated virus rep68 fusion protein has DNA-RNA helicase and ATPase activities. J. Virol. 69: 3542-3548.

41. Weitzman, M. D., S. R. M. Kyöstiö, R. M. Kotin, and R. A. Owens 1994. Adeno-associated virus (AAV) rep proteins mediate complex formation between AAV DNA and its integration site in human DNA. Proc. Natl. Acad. Sci. USA 91: 5808-5812.

42. Deuschle, U., W. K-H Meyer, and H-J Thiesen. 1995. Tetracycline-reversible silencing of eukaryotic promoters. Mol. Cell. Biol. 15: 1907-1914.

43. Robertson, E. J. 1987. Teratocarcinomas and embryonic stem cells: A practical Approach. IRL Press, Oxford. pp. 71-182.

44. Pittman, D. D., E. M. Alderman, K. N. Tomkinson, J. H. Wang, A. R. Giles, and R. J. Kaufman. 1993. Biochemical, Immunological, and in vivo characterization of B-domain-deleted factor VIII. Blood 81: 2925-2935.

45. Ghebranious, N., B. J. Knoll, L. Yavorkovsky, Z. Ilic, J. Papaconstantinou, G. Lozano, and S. Sell. 1995. Developmental control of transcription of the CAT reporter gene by a truncated mouse alphafetoprotein gene regulatory region in transgenic mice. Ma. Reprod. Dev. 42: 1-6.

46. Spear, B. T., and S. M. Tilghman. 1990. Role of alpha-fetoprotein regulatory elements in transcriptional activation in transient heterokaryons. Mol. Cell. Biol. 10: 5047-5054.

47. Feuerman, M. H., R. Godbout, R. S. Ingram, and S. M. Tilgman. 1989. Tissue-specific transcription of the mouse alpha-fetoprotein gene promoter is dependent on HNF-1. Mol. Cell. Biol. 9: 4204-4212.

48. Vogt, T. F., R. S. Compton, R. W. Scott, and S. Tilghman. 1988. Differential requirements for cellular enhancers in stem and differentiated cells. Nucleic Acids Res. 16: 487-500.

49. Mittereder, N., K. L. March, and B. C. Trapnell. 1996. Evaluation of the concentration and bioactivity of adenovirus vectors for gene therapy. J. Viol. 70: 7498-7509.

50. Kotin, K., R. Linden, and K. Berns. 1992. Characterization of a preferred site on human chromosome 19q for integration of adeno-associated virus DNA by non-homologous recombination. EMBO J. 11: 5071-5078.

51. Bi, L., A. Lawler, S. Antonarakis, K. High, J. Gearhart, and H. Kazazian. 1995. Targeted disruption of the mouse factor VIII gene produces a model of Hemophilia A. Nature Genetics 10: 119-121.

52. Mizushimia and Nagata. 1990. pEF-BOS, a powerful mammalian expression vector. Nuc. Acids Res. 18:5322.

53. Fay, P. J. 1993. FVIII structure and function. Thromb. Haemostas. 70: 63-67.

54. McGrory, et al. 1988. A simple technique for rescue of early region I mutations into infectious human adenovirus type 5. Virology, 163: 614-617.

55. “The Metabolic and Molecular Bases of Inherited Diseases” (Editors: C. R. Scriver, A. L. Beaudet, W. S. Sly, and D. Valle, 7th edition, 1996).

56. Balague, C., Kalla, M. an Zhang, W-W. (1997) Adeno-associated Rep 78 protein and terminal repeats enhance integration of DNA sequences into the cellular genome. J. Virol. 71: 3299-3306.

57. Gendron-Maguire, M. and T. Gridley. 1993. Identification of transgenic mice. Methods in Enzymology 225: 794-799.

58. Mann, J. R. 1993. Surgical techniques in production of transgenic mice. Methods in Enzymology 225: 782-793.

59. Gordon, J. W. 1993. Production of transgenic mice. Methods in Enzymology 225: 747-771.

60. Mann, J. R. and A. P. McMahon. 1993. Factors influencing frequency production of transgenic mice.

61. Bradley, A. 1987. Production and analysis of chimaeric mice. In Teratocarcinomas and embryonic stem cells—a practical approach. Pp. 113-151. Robertson, E. J., Ed. IRL Press, Washington, D.C.

62. Koeberl, et al. 1995. Sequences within the coding regions of clotting factor VIII and CFTR block transcriptional elongation. Human Gene Ther. 6: 469-479.

63. Hoeben, et al. 1995. Expression of the blood-clotting factor FVIII cDNA is repressed by a transcriptional silencer located in its coding region. Blood 85:2447-2454.

64. Miller, A. D. and G. J. Rosman. 1989. Improved retroviral vectors for gene therapy and expression. Biotechniques 7: 980-990.

65. Mulligan, R. C. 1993. The basic science of gene therapy. Science 260: 926-932.

66. Parks, et al. 1996. A helper-dependent adenovirus helper system: removal of helper virus by cre-mediated excision of the viral packaging signal. Proc. Natl. Acad. Sci. USA 93: 13565-13570.

67. Hitt, et al. In Methods in Molecular Genetics, K. W. Adolph, ed. (Academic Press, San Diego, Calif.) Vol. 7, pp. 13-30.

68. Broach, et al. 1982. Recombination within the yeast plasmid 2 mu circle is site-specific. Cell 29: 227-234.

69. Gorman, et al. 1982. Recombinant genomes which express chloramphenicol acetyltransferase in mammalian cells. Mol. Cell. Biol. 2: 1044-1051.

70. Calos, M. P. 1996. The potential of extrachromosomal replicating vectors for gene therapy. TIG 12: 463-466.

71. Krysan, et al. 1993. Autonomous replication in human cells of multimers of specific human and bacterial DNA sequences. Mol. Cell. Biol. 13: 2688-2696.

72. Haase, S. B. and M. P. Calos. 1991. Replication control of autonomously replicating human sequences. Nucleic Acids Res. 19: 5053-5058.

73. Kay, et al. 1993. In vivo gene therapy of hemophilia B: sustained partial correction in factor IX-deficient dogs. Science 262: 117-119.

74. Chuah, et al. 1995. Development and analysis of retroviral vectors expressing human factor VIII as a potential gene therapy for hemophilia A. Human Gene Ther. 6: 1363-1377.

75. Hammarskjold, M. -L. and G. Winberg. 1980. Encapsidation of adenovirus 16 DNA is directed by a small DNA sequence at the left end of the genome. Cell 20: 787-795.

76. Yang, Y., et al. 1994. Cellular immunity to viral antigen limits E1-deleted adenoviruses for gene therapy. Proc. Natl. Acad Sci. U.S.A. 91: 4407-4411.

77. Dwarki, V. J., et al. 1995. Gene therapy for hemophilia A: Production of therapeutic levels of human factor VIII in vivo in mice. Proc. Natl. Acad. Sci. USA 92: 1023-1027.

78. Dai, et al. 1992. Gene therapy via primary myoblasts: Long-term expression of factor IX protein following transplantation in vivo. Proc. Natl. Acad. Sci. USA 89: 10892-10895.

79. Cotton, M., et al. 1992. High-efficiency receptor-mediated delivery of small and large (48 kilobase) gene constructs using the endosome disruption activity of defective or chemically-inactivated adenovirus particles. Proc. Natl. Acad. Sci. USA 89: 6094-6098.

80. Frank, et al. 1994. High-level expression of various apolipoprotein(a) isoforms by “transferinfection”: the role of kringle IV sequences in the extracellular association with low-density lipoprotein. Biochemistry 3: 12329-12339.

81. Chen, C. and H. Okayama. 1987. High-efficieny transformation of mammalian cells by plasmid DNA. Mol. Cell. Biol. 7: 2745-2752.

82. Marchioro, T. L., et al. 1969. Science 163:188.

83. Webster, et al. 1971. Am. J. Physiology 220:1147.

84. Graham, F. L., and L. Prevec. 1991. Manipulation of adenovirus vectors. In: Methods in Molecular Biology (Vol. 7), Gene Transfer and Expression Protocols, ed. E. J. Murray, The Humana Press Inc., Clifton, N.J.

85. Engelhardt, J. F., X. Ye, B. Doranz, and J. M. Wilson. 1994. Ablation of E2A in recombinant adenoviruses improves transgene persistence and decreases inflammatory response in mouse liver. Proc. Natl. Acad. Sci. 91: 6196-6200.

86. Krouglik, V., and F. L. Graham. 1995. Development of cell lines capable of complementing E1, E4, and protein IX defective adenovirus type 5 mutants. Human Gene Ther. 6: 1575-1586.

87. Mitani, K., F. L. Graham, C. T. Caskey, and S. Kochanek. 1995. Rescue, propagation, and patial purification of a helper virus-dependent adenovirus vector. Proc. Natl. Acad. Sci. 92: 3854-3858.

88. Lieber, et al. 1996. Recombinant adenoviruses with large deletions generated by Cre-mediated excision exhibit different biological properties compared with first-generation vectors in vitro and in vivo. J. Virology 70:894-8960.

89. Stow, N. D. 1981. Cloning of a DNA fragment from the bleft-hand terminus of the adenoviruse type 2 genome and its use in site-directed mutagenesis J. Virol. 37, 171-180.

90. Grable, M., and P. Hearing. 1992. cis and trans requirements for the selective packaging of adenovirus type 5 DNA. J. Virol. 66: 723-731.

91. Graham, F. L., and L. Prevec. 1991. Manipulation of adenovirus vectors. In: Methods in Molecular Biology (Vol. 7), Gene Transfer and Expression Protocols, ed. E. J. Murray, The Humana Press Inc., Clifton, N.J.

92. Graham, F. L. 1984. Covalently closed circles of human adenovirus DNA. EMBO J. 3: 2917-2922.

93. Ruben, M., S. Bacchetti, and F. L. Graham 1983. Nature 301: 172-174.

94. McGrory, W. J., D. S. Bautista, and F. L. Graham 1988A simple technique for the rescue of early region I mutations into infectious human adenovirus type 5. Viology 163: 614-617.

95. Robinson, A. J., H. B. Younghusband, and A. J. D. Bellett. 1973. A circular DNA-protein complex from adenovirus. Virol. 56: 54

96. Bett, A. J., W. Haddara, L. Prevec, and F. L. Graham 1994. An efficient and flexible system for construction of adenovirus vectors with insertions or deletions in early regions 1 and 3. Proc. Natl. Acad. Sci. USA 91: 8802-8806.

97. Grable, M., and P. Hearing. 1990. Adenovirus type 5 packaging domain is composed of a repeated element that is functionally redundant. J. Virol. 64: 2047-2056.

98. Webster, N., J. R. Jin, S. Green, M. Hollis, and P. Chambon 1988. The yeast UAS_{G} is a transcriptional enhancer in human Hela cells in the presence of the Ga14 trans-activator. Cell 52: 169-178,

99. Gatz, C. and P. H. Ouau 1988. Tn10-encodes tet repressor can regulate an operator-containing plant promoter. Proc. Natl. Acad. Sci. USA 85: 1394-1397.

100. Gossen, M. and H. Bujard 1992. Tight control of gene expression in mammalian cells by tetracycline-responsive promoters. Proc. Natl. Acad. Sci. USA 89: 5547-5551.

101. Nevins, J. R. 1993. Transcriptional activation by the adenovirus E1A proteins. Semin. Viol. 4: 25-31.

102. Graham, F. L., P. J. Abrahams, C. Mulder, H. I. Heijneker, S. O. Warnaar, F. A. J. de Vries, W. Fiers, and A. J. van der Eb 1974. Studies on In Vitro Transformation by DNA and DNA Fragments of Human Adenoviruses and Simian Virus 40. Cold Spring Harbor Symp. Quant. Biol. 39: 637-650.

103. Mckinnon, R. D., S. Bacchetti, and F. L. Graham 1982. Tn5 Mutagenesis of the Transforming Genes of Human Adenovirus Type 5. Gene 19: 33-42.

104. van der Eb, A. J., C. Mulder, F. L. Graham, and A. Houweling 1977. Transformation With Specific Fragments of Adenovirus DNAs. I. Isolation of Specific Fragments With Transforming Activity of Adenovirus 2 and 5 DNA. Gene 2: 115-132.

105. Graham, F. L., J. Smiley, W. C. Russell, and R. Nairn. 1977. Characteristics of a human cell line transformed by DNA from human adenovirus type 5. J. Gen. Virol. 36: 59-72.

106. Aiello, L., R. Guilfoyle, K. Huebner, and R. Weinmann 1979. Adenovirus 5 DNA Sequences Present and RNA Sequences Transcribed in Transformed Human Embryo Kidney Cells (HEK-Ad-5 or 293). Virology 94: 460-469.

107. Lochmuller, H., A. Jani, J. Huard, S. Prescott, M. Simoneau, B. Massie, G. Karpati, and G. Ascadi, G. 1994. Emergence of Early Region 1-Containing, Replication-Competent Adenovirus in Stocks of Replication Defective Adenovirus Recombinants (E1+E3 Deletions) During Multiple Passages in 293 Cells. Human Gene Ther. 5: 1485-1491.

108. Fallaux, F. J., O. Kranenburg, S. J. Cramer, A. Houweling, H. van Ormondt, R. C. Hoeben, and A. J. van der Eb 1996. Characterization of 911: A new Helper Cell Line for the Titration and Propagation of Early Region 1—Deleted Adenoviral Vectors. Human Gene Ther. 7: 215-222.

109. Imler, J -L., C. Chartier, D. Dreyer, A. Dieterle, M. Sainte-Marie, T. Faure, A. Pavirani, and M. Mehtali 1996. Novel Complementation Cell Lines Derived From Human Lung Carcinoma A549 Cells Support the Growth of E1-Deleted Adenovirus Vectors. Gene Ther. 3: 75-84.

110. Miller, S. A., D. D. Dykes, and H. F. Polesky 1988. A simple salting out procedure for extracting DNA from human nucleated cells. Nucl. Acid. Res. 16: 1215.

111. Kilby, N. J., M. R. Snaith, and J. A. H. Murray. 1993. Site-specific recombinases: tools for genome engineering. Trends Genet. 9: 413-421.

112. Austin, S., M. Ziese, and N. Sternberg. 1981. A novel role for site-specific recombination in maintenance of bacterial replicons. Cell 25: 729-36.

113. Broach J. R., V. R. Guarascio, and M. Jayaram. 1982. Recombination within the yeast plasmid 2 mu circle is site-specific. Cell 29: 227-34.

114. Wobbe C. R., F. Dean, L. Weissbach, and J. Hurwitz. 1985. In vitro replication of duplex circular DNA containing the simian virus 40 DNA origin site. Proc. Natl. Acad. Sci. USA, 82: 5710-4.

115. Yates J. L., N. Warren, and B. Sugden. 1985. Stable replication of plasmids derived from Epstein-Barr virus in various mammalian cells. Nature 313: 812-5.

116. Yates, J. L., and N. Guan. 1991. Epstein-Barr Virus-derived plasmids replicate only once per cell cycle and are not amplified after entry into cells. J. Virol. 65: 483-488.

117. Miller, A. R., W. H. McBride, K. Hunt, and J. S. Economou. 1994. Cytokine-mediated gene therapy for cancer. Ann. of Surg. Onc. 1:436-450.

118. Rosenberg, S. A., M. T. Lotze, J. C. Tang, P. M. Aebersold, W. M. Linehan, C. A. Deipp, and D. E. White. 1989. Experience with the use of high-dose interleukin-2 in the treatment of 652 cancer patients. Ann. Surg. 210:474-484.

119. Dranoff, G. and R. C. Mulligan. 1995. Gene transfer as cancer therapy. Adv. in Immunol., 58:417-454.

120. Isaka, Y., D. K. Brees, K. Ikegaya, T. Kaneda, E. Imai, N. A. Noble, And W. A. Border. 1996. Gene therapy by skeletal muscle expression of decorin prevents fibrotic disease in rat kidney. Nat. Med. 1996, 2:418-423.

121. Fakhrai, H., O. Dorigo, D. L. Shawler, H. Lin, D. Mercola, K. L. Black, I. Royston, and R. E. Sobol. 1996 Eradication of established intracranial rat gliomas by transforming growth factor b antisense gene therapy. Proc. Natl. Acad. Sci. U.S.A. 93:2909-2914.

122. Leach, D. R., M. F. Krummel and J. P. Allison. 1996. Enhancement of antitumor immunity by CTLA-4 blockade. Science 271:1734-1736.

123. Springer, T. A. 1990. Adhesion receptors of the immune system. Nature. 346:425-434.

124. Lenschow, D. J., T. L. Walunas, and J. A. Bluestone. 1996. CD28/B7 system of T cell costimulation. Annu. Rev. Immunol. 14: 233-58.

125. Zhang, W. -W. 1995. Adenovirus as a system for delivering and expressing tumor suppressor genes in tumor cells. Method: A Companion to Methods in Enzymology 8: 198-214.

126. Harris, H. 1993. How tumor suppressor genes were discovered. FASEB J. 7: 978-979.

127. Kamb, A., N. A. Gruis, J. Weaver-Feldhaus, Q. Liu, K. Harshman, S. V. Tavtigian, E. Stockert, R. S. Day III, B. E. Johnson, and M. H. Skolnick. 1994. A cell cycle regulator potentially involved in genesis of many tumor types. Science 264: 436-440.

128. Nobori, T., K. Miura, D. J. Wu, A. Lois, K. Takabayashi, and D. A. Carson. 1994. Deletions of the cyclin-dependent kinase-4 inhibitor gene in multiple human cancers. Nature 368: 753-756.

129. Miki, Y., J. Swensen, D. Shattuck-Eidens, P. A. Futreal, K. Harshman, S. Tavtigian, Q. Liu, C. Cochran, L. M. Bennett, W. Ding, R. Bell, J. Rosenthal, C. Hussey, T. Tran, M. McClure, C. Frye, T. Hattier, R. Phelps, A. Haugen-Strano, H. Katcher, K. Yakumo, Z. Gholami, D. Shaffer, S. Stone, S. Bayer, C. Wray, R. Bogden, P. Dayananth, J. Ward, P. Tonin, S. Narod, P. K. Bristow, F. H. Norris, L. Helvering, P. Morrison, P. Rosteck, M. Lai, J. C. Barrett, C. Lewis, S. Neuhausen, L. Cannon-Albright, D. Goldgar, R. Wiseman, A. Kamb, and M. H. Skolnick. 1994. A strong candidate for the breast and ovarian cancer susceptibility gene BRCA1. Science 266: 66-71.

130. Hannon G., and D. Beach. 1994. p15INK4B is a potential effector of TGF-βinduced cell cycle arrest. Nature 371: 257-261.

131. Zhang, W. -W., and X. Fang. 1995. Gene therapy strategies for cancer. Exp. Opin. Invest. Drugs 4: 487-514.

132. International Patent Application number PCT/US96/05310, published Oct. 24, 1996 as WO 96/33280.

133. International Patent Application numver PCT/US96/14312, published Mar. 13, 1997 as WO 97/09442.

134. International Patent Application numver PCT/GB96/02061, published Mar. 6, 1997 as WO 97/08330.

We claim:

1. A minimal adenovirus vector comprising one or more human immunodeficiency virus (HIV) genes.

2. The vector of claim 1, wherein the gene or genes are selected from the group consisting of the gag, pol, tat^{nf}, and rev genes.

3. The vector of claim 1, wherein the gene or genes are under the control of a heterologous promoter.

4. The vector of claim 3, wherein the promoter is the CMV promoter.

5. The vector of claim 1, further comprising one or more coding sequences for GM-CSF.

6. The vector of claim 5, wherein the coding sequence or coding sequences are under the control of a heterologous promoter.

7. The vector of claim 6, wherein the promoter is the RSV promoter.

8. A minimal adenovirus vector comprising one or more human papilloma virus (HPV) genes.

9. The vector of claim 8, wherein the gene or genes are selected from the group consisting of the genes in the HPV L1 gene region, the genes in the HPV L2 gene region, HPV E6 and HPV E7.

10. The vector of claim 8, wherein the gene or genes are under the control of a heterologous promoter.

11. The vector of claim 10, wherein the promoter is selected from the group consisting of the SV40 promoter and the TK promoter.

12. The vector of claim 8, further comprising one or more GM-CSF coding sequences.

13. The vector of claim 12, wherein the coding sequence or coding sequences are under the control of a heterologous promoter.

14. The vector of claim 13, wherein the promoter is the RSV promoter.


SEQUENCE LISTING (9 sequences)

SEQ ID NO: 1 - DNA, 23 bp, Artificial Sequence
  FVIII primer #1
accagtcaaagggagaaagaaga

SEQ ID NO: 2 - DNA, 23 bp, Artificial Sequence
  FVIII primer #2
cgatggttcctcacaagaaatgt

SEQ ID NO: 3 - DNA, 20 bp, Artificial sequence
  packaging signal primer +1901
ggaacacatgtaagcgacgg

SEQ ID NO: 4 - DNA, 34 bp, Artificial Sequence
  packaging signal primer +1902
ccatcgataataataaaacgccaactttgacccg

SEQ ID NO: 5 - DNA, 23 bp, Artificial Sequence
  PCR primer U2492
gctgtctggtgcgtttcactgat

SEQ ID NO: 6 - DNA, 23 bp, Artificial Sequence
  PCR primer L2722
tcacaaagggagttttccacacg

SEQ ID NO: 7 - DNA, 17 bp, Saccharomyces cerevisiae
  misc_feature (1)..(17): GAL 4 DNA binding sequence
cggagtactgtcctccg

SEQ ID NO: 8 - DNA, 17 bp, Saccharomyces cerevisiae
  misc_feature (1)..(17): GAL 4 DNA binding sequence
cggaggactgtcctccg

SEQ ID NO: 9 - DNA, 19 bp, Escherichia coli
  misc_feature (1)..(19): tetR DNA binding sequence
tccctatcagtgatagaga